SEQUENCE LISTING (198 sequences)

SEQ ID NO: 1 - protein, 145 aa, Acinetobacter soli
MNAQKGFTLIELMIVIAIIGILAAIAIPAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGT

SEQ ID NO: 2 - protein, 147 aa, Acinetobacter sp.
MNAQKGFTLIELMIVIAIIGILAAIAIPAYTDYTVRARVSEGLTAASSMKTTVSENILNAGALVAGTPSTAGSSCVGVQEISASNATTNVATATCGASSAGQIIVTMDTTKAKGANITLTPTYASGAVTWKCTTTSDKKYVPSECRG

SEQ ID NO: 3 - protein, 146 aa, Acinetobacter soli
MNAQKGFTLIELMIVIAIIGILAAIAIPAYTDYTVRARVSEGLTTASAMKATVSENILSAGQIVTGTPSTANSSCVGVQEINASSSTSNVATATCSGLGVITVTMDSTKAKGVNLTLTPTYTTSNAVTWKCTTTSDKKYVPSECRN

SEQ ID NO: 4 - protein, 142 aa, Acinetobacter radioresistens
MNTQKGFTLIELMIVIAIIGILAAIAIPAYTDYTVRARVTEAVSTASSMKATVSENIMNAGGTQIPTSGNCVGVQTIAASNATKNVATATCTDSTGVIVVTTTPAAKSVPLTLTPTYTGGNVKWACSTTANFKNYVPSECRS

SEQ ID NO: 5 - protein, 140 aa, Acinetobacter sp.
MNAQKGFTLIELMIVIAIIGILAAIAIPAYTDYTVRARVTEALTTASAMKATVSENIMSAGGTTIASSACNGVISASATTNVASSACSGSGVISVTTTAAAKGIVLTLTPKYTGGNVAWQCTTTSGDAQKYVPSECRTTS

SEQ ID NO: 6 - protein, 150 aa, Acinetobacter sp.
MNTQKGFTLIELMIVIAIIGILAAIAIPAYTDYTVRAKVTEAISTASAMKATVSENLMSAGGTSIVSTNANCAGVETIGASNKTKNVESAACTAATGVILVTTTAEAKSVPLTLKPTYTGSNVQWKCGTTAAAFKYVPSECRNDSSGTGF

SEQ ID NO: 7 - protein, 140 aa, Artificial Sequence
  Description of Artificial Sequence Synthetic polypeptide
MNAQKGFTLIELMIVIAIIGILAAIALPAYTDYTTRARVSEALTTASAMKATVSENIISKGGTSIDEDSACIGVATVGSDASAATKNVQKSVCDKGVITVTTTPDAKSVPLILTPSYSGDGVEWTCTTTADKKYVPAECR

SEQ ID NO: 8 - protein, 143 aa, Artificial Sequence
  Description of Artificial Sequence Synthetic polypeptide
MNTAQKGFTLIELMIVIAIIGILAAIAIPAYSDYTARARVTEAVTTASSMKATVSENIISKGGTTIGAGSCAGVSLIGASNKTKNVLSSTCTDTTGVILVTTTADAKSVPLTLTPTYTGDAVTWKCTTTSDFTKYVPAECRPH

SEQ ID NO: 9 - protein, 117 aa, Acinetobacter soli
AYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGT

SEQ ID NO: 10 - protein, 119 aa, Acinetobacter sp.
AYTDYTVRARVSEGLTAASSMKTTVSENILNAGALVAGTPSTAGSSCVGVQEISASNATTNVATATCGASSAGQIIVTMDTTKAKGANITLTPTYASGAVTWKCTTTSDKKYVPSECRG

SEQ ID NO: 11 - protein, 118 aa, Acinetobacter soli
AYTDYTVRARVSEGLTTASAMKATVSENILSAGQIVTGTPSTANSSCVGVQEINASSSTSNVATATCSGLGVITVTMDSTKAKGVNLTLTPTYTTSNAVTWKCTTTSDKKYVPSECRN

SEQ ID NO: 12 - protein, 114 aa, Acinetobacter radioresistens
AYTDYTVRARVTEAVSTASSMKATVSENIMNAGGTQIPTSGNCVGVQTIAASNATKNVATATCTDSTGVIVVTTTPAAKSVPLTLTPTYTGGNVKWACSTTANFKNYVPSECRS

SEQ ID NO: 13 - protein, 112 aa, Acinetobacter sp.
AYTDYTVRARVTEALTTASAMKATVSENIMSAGGTTIASSACNGVISASATTNVASSACSGSGVISVTTTAAAKGIVLTLTPKYTGGNVAWQCTTTSGDAQKYVPSECRTTS

SEQ ID NO: 14 - protein, 122 aa, Acinetobacter sp.
AYTDYTVRAKVTEAISTASAMKATVSENLMSAGGTSIVSTNANCAGVETIGASNKTKNVESAACTAATGVILVTTTAEAKSVPLTLKPTYTGSNVQWKCGTTAAAFKYVPSECRNDSSGTGF

SEQ ID NO: 15 - protein, 140 aa, Artificial Sequence; identical to SEQ ID NO: 7
  Description of Artificial Sequence Synthetic polypeptide

SEQ ID NO: 16 - protein, 143 aa, Artificial Sequence; identical to SEQ ID NO: 8
  Description of Artificial Sequence Synthetic polypeptide

SEQ ID NO: 17 - protein, 21 aa, Artificial Sequence
  Description of Artificial Sequence Synthetic peptide
  MOD_RES (1)..(1): V, T, A, or I
  MOD_RES (4)..(4): Q, T, E, A, or S
  MOD_RES (5)..(5): E, Q, T, or L
  MOD_RES (6)..(6): I or V
  MOD_RES (7)..(7): S, N, A, or G
  MOD_RES (8)..(8): S or absent
  MOD_RES (9)..(9): G, D, or absent
  MOD_RES (12)..(12): N, S, or A
  MOD_RES (13)..(13): A, S, or K
  MOD_RES (15)..(15): T, S, or K
  MOD_RES (18)..(18): A, E, Q, or L
  MOD_RES (19)..(19): T, S, or K
  MOD_RES (20)..(20): A or S
  MOD_RES (21)..(21): T, Q, A, or V
XGVXXXXXXASXXTXNVXXXX

SEQ ID NO: 18 - protein, 612 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLTILGWPLAERTVVIPSAIPTDPRNVGGDLDPSSIPDKEQAISALPDYASQPGKPPREDLK

SEQ ID NO: 19 - protein, 637 aa, Artificial Sequence
  Description of Artificial Sequence Synthetic polypeptide
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLTILGWPLAERTVVIPSAIPTDPRNVGGDLDPSSIPDKEQAISALPDYASQPGKPPREDLKHHHHHH

SEQ ID NO: 20 - protein, 637 aa, Artificial Sequence; identical to SEQ ID NO: 19
  Description of Artificial Sequence Synthetic polypeptide

SEQ ID NO: 21 - protein, 637 aa, Artificial Sequence; identical to SEQ ID NO: 19
  Description of Artificial Sequence Synthetic polypeptide

SEQ ID NO: 22 - protein, 637 aa, Artificial Sequence; identical to SEQ ID NO: 19
  Description of Artificial Sequence Synthetic polypeptide

SEQ ID NO: 23 - protein, 637 aa, Artificial Sequence; identical to SEQ ID NO: 19
  Description of Artificial Sequence Synthetic polypeptide

SEQ ID NO: 24 - protein, 535 aa, Artificial Sequence
  Description of Artificial Sequence Synthetic polypeptide
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 25 - protein, 560 aa, Artificial Sequence
  Description of Artificial Sequence Synthetic polypeptide
MIKFLSALILLLVTTAAQAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSHHHHHH

SEQ ID NO: 26 - protein, 560 aa, Artificial Sequence; identical to SEQ ID NO: 25
  Description of Artificial Sequence Synthetic polypeptide

SEQ ID NO: 27 - protein, 560 aa, Artificial Sequence; identical to SEQ ID NO: 25
  Description of Artificial Sequence Synthetic polypeptide

SEQ ID NO: 28 - protein, 560 aa, Artificial Sequence; identical to SEQ ID NO: 25
  Description of Artificial Sequence Synthetic polypeptide

SEQ ID NO: 29 - protein, 560 aa, Artificial Sequence; identical to SEQ ID NO: 25
  Description of Artificial Sequence Synthetic polypeptide

SEQ ID NO: 30 - protein, 23 aa, Artificial Sequence
  Description of Artificial Sequence Synthetic peptide
CTGVTQIASGASAATTNVASAQC

SEQ ID NO: 31 - protein, 23 aa, Artificial Sequence
  Description of Artificial Sequence Synthetic peptide
STGVTQIASGASAATTNVASAQS

SEQ ID NO: 32 - protein, 22 aa, Artificial Sequence
  Description of Artificial Sequence Synthetic peptide
CTGVTQIASGASAATTNVASAQ

SEQ ID NO: 33 - protein, 21 aa, Artificial Sequence
  Description of Artificial Sequence Synthetic peptide
CTGVTQIASGASAATTNVASA

SEQ ID NO: 34 - protein, 20 aa, Artificial Sequence
  Description of Artificial Sequence Synthetic peptide
CTGVTQIASGASAATTNVAS

SEQ ID NO: 35 - protein, 19 aa, Artificial Sequence
  Description of Artificial Sequence Synthetic peptide
CTGVTQIASGASAATTNVA

SEQ ID NO: 36 - protein, 18 aa, Artificial Sequence
  Description of Artificial Sequence Synthetic peptide
CTGVTQIASGASAATTNV

SEQ ID NO: 37 - protein, 17 aa, Artificial Sequence
  Description of Artificial Sequence Synthetic peptide
CTGVTQIASGASAATTN

SEQ ID NO: 38 - protein, 16 aa, Artificial Sequence
  Description of Artificial Sequence Synthetic peptide
CTGVTQIASGASAATT

SEQ ID NO: 39 - protein, 15 aa, Artificial Sequence
  Description of Artificial Sequence Synthetic peptide
CTGVTQIASGASAAT

SEQ ID NO: 40 - protein, 14 aa, Artificial Sequence
  Description of Artificial Sequence Synthetic peptide
CTGVTQIASGASAA

SEQ ID NO: 41 - protein, 13 aa, Artificial Sequence
  Description of Artificial Sequence Synthetic peptide
CTGVTQIASGASA

SEQ ID NO: 42 - protein, 12 aa, Artificial Sequence
  Description of Artificial Sequence Synthetic peptide
CTGVTQIASGAS

SEQ ID NO: 43 - protein, 22 aa, Artificial Sequence
  Description of Artificial Sequence Synthetic peptide
TGVTQIASGASAATTNVASAQC

SEQ ID NO: 44 - protein, 21 aa, Artificial Sequence
  Description of Artificial Sequence Synthetic peptide
TGVTQIASGASAATTNVASAQ

SEQ ID NO: 45 - protein, 20 aa, Artificial Sequence
  Description of Artificial Sequence Synthetic peptide
TGVTQIASGASAATTNVASA

SEQ ID NO: 46 - protein, 19 aa, Artificial Sequence
  Description of Artificial Sequence Synthetic peptide
TGVTQIASGASAATTNVAS

SEQ ID NO: 47 - protein, 18 aa, Artificial Sequence
  Description of Artificial Sequence Synthetic peptide
TGVTQIASGASAATTNVA

SEQ ID NO: 48 - protein, 17 aa, Artificial Sequence
  Description of Artificial Sequence Synthetic peptide
TGVTQIASGASAATTNV

SEQ ID NO: 49 - protein, 16 aa, Artificial Sequence
  Description of Artificial Sequence Synthetic peptide
TGVTQIASGASAATTN

SEQ ID NO: 50 - protein, 15 aa, Artificial Sequence
  Description of Artificial Sequence Synthetic peptide
TGVTQIASGASAATT

SEQ ID NO: 51 - protein, 14 aa, Artificial Sequence
  Description of Artificial Sequence Synthetic peptide
TGVTQIASGASAAT

SEQ ID NO: 52 - protein, 13 aa, Artificial Sequence
  Description of Artificial Sequence Synthetic peptide
TGVTQIASGASAA

SEQ ID NO: 53 - protein, 12 aa, Artificial Sequence
  Description of Artificial Sequence Synthetic peptide
TGVTQIASGASA

SEQ ID NO: 54 - protein, 11 aa, Artificial Sequence
  Description of Artificial Sequence Synthetic peptide
TGVTQIASGAS

SEQ ID NO: 55 - protein, 21 aa, Artificial Sequence
  Description of Artificial Sequence Synthetic peptide
GVTQIASGASAATTNVASAQC

SEQ ID NO: 56 - protein, 20 aa, Artificial Sequence
  Description of Artificial Sequence Synthetic peptide
GVTQIASGASAATTNVASAQ

SEQ ID NO: 57 - protein, 19 aa, Artificial Sequence
  Description of Artificial Sequence Synthetic peptide
GVTQIASGASAATTNVASA

SEQ ID NO: 58 - protein, 18 aa, Artificial Sequence
  Description of Artificial Sequence Synthetic peptide
GVTQIASGASAATTNVAS

SEQ ID NO: 59 - protein, 17 aa, Artificial Sequence
  Description of Artificial Sequence Synthetic peptide
GVTQIASGASAATTNVA

SEQ ID NO: 60 - protein, 16 aa, Artificial Sequence
  Description of Artificial Sequence Synthetic peptide
GVTQIASGASAATTNV

SEQ ID NO: 61 - protein, 15 aa, Artificial Sequence
  Description of Artificial Sequence Synthetic peptide
GVTQIASGASAATTN

SEQ ID NO: 62 - protein, 14 aa, Artificial Sequence
  Description of Artificial Sequence Synthetic peptide
GVTQIASGASAATT

SEQ ID NO: 63 - protein, 13 aa, Artificial Sequence
  Description of Artificial Sequence Synthetic peptide
GVTQIASGASAAT

SEQ ID NO: 64 - protein, 12 aa, Artificial Sequence
  Description of Artificial Sequence Synthetic peptide
GVTQIASGASAA

SEQ ID NO: 65 - protein, 11 aa, Artificial Sequence
  Description of Artificial Sequence Synthetic peptide
GVTQIASGASA

SEQ ID NO: 66 - protein, 10 aa, Artificial Sequence
  Description of Artificial Sequence Synthetic peptide
GVTQIASGAS

SEQ ID NO: 67 - protein, 20 aa, Artificial Sequence
  Description of Artificial Sequence Synthetic peptide
VTQIASGASAATTNVASAQC

SEQ ID NO: 68 - protein, 19 aa, Artificial Sequence
  Description of Artificial Sequence Synthetic peptide
VTQIASGASAATTNVASAQ

SEQ ID NO: 69 - protein, 18 aa, Artificial Sequence
  Description of Artificial Sequence Synthetic peptide
VTQIASGASAATTNVASA

SEQ ID NO: 70 - protein, 17 aa, Artificial Sequence
  Description of Artificial Sequence Synthetic peptide
VTQIASGASAATTNVAS

SEQ ID NO: 71 - protein, 16 aa, Artificial Sequence
  Description of Artificial Sequence Synthetic peptide
VTQIASGASAATTNVA

SEQ ID NO: 72 - protein, 15 aa, Artificial Sequence
  Description of Artificial Sequence Synthetic peptide
VTQIASGASAATTNV

SEQ ID NO: 73 - protein, 14 aa, Artificial Sequence
  Description of Artificial Sequence Synthetic peptide
VTQIASGASAATTN

SEQ ID NO: 74 - protein, 13 aa, Artificial Sequence
  Description of Artificial Sequence Synthetic peptide
VTQIASGASAATT

SEQ ID NO: 75 - protein, 12 aa, Artificial Sequence
  Description of Artificial Sequence Synthetic peptide
VTQIASGASAAT

SEQ ID NO: 76 - protein, 11 aa, Artificial Sequence
  Description of Artificial Sequence Synthetic peptide
VTQIASGASAA

SEQ ID NO: 77 - protein, 10 aa, Artificial Sequence
  Description of Artificial Sequence Synthetic peptide
VTQIASGASA

SEQ ID NO: 78 - protein, 9 aa, Artificial Sequence
  Description of Artificial Sequence Synthetic peptide
VTQIASGAS

SEQ ID NO: 79 - protein, 19 aa, Artificial Sequence
  Description of Artificial Sequence Synthetic peptide
TQIASGASAATTNVASAQC

SEQ ID NO: 80 - protein, 18 aa, Artificial Sequence
  Description of Artificial Sequence Synthetic peptide
TQIASGASAATTNVASAQ

SEQ ID NO: 81 - protein, 17 aa, Artificial Sequence
  Description of Artificial Sequence Synthetic peptide
TQIASGASAATTNVASA

SEQ ID NO: 82 - protein, 16 aa, Artificial Sequence
  Description of Artificial Sequence Synthetic peptide
TQIASGASAATTNVAS

SEQ ID NO: 83 - protein, 15 aa, Artificial Sequence
  Description of Artificial Sequence Synthetic peptide
TQIASGASAATTNVA

SEQ ID NO: 84 - protein, 14 aa, Artificial Sequence
  Description of Artificial Sequence Synthetic peptide
TQIASGASAATTNV

SEQ ID NO: 85 - protein, 13 aa, Artificial Sequence
  Description of Artificial Sequence Synthetic peptide
TQIASGASAATTN

SEQ ID NO: 86 - protein, 12 aa, Artificial Sequence
  Description of Artificial Sequence Synthetic peptide
TQIASGASAATT

SEQ ID NO: 87 - protein, 11 aa, Artificial Sequence
  Description of Artificial Sequence Synthetic peptide
TQIASGASAAT

SEQ ID NO: 88 - protein, 10 aa, Artificial Sequence
  Description of Artificial Sequence Synthetic peptide
TQIASGASAA

SEQ ID NO: 89 - protein, 9 aa, Artificial Sequence
  Description of Artificial Sequence Synthetic peptide
TQIASGASA

SEQ ID NO: 90 - protein, 8 aa, Artificial Sequence
  Description of Artificial Sequence Synthetic peptide
TQIASGAS

SEQ ID NO: 91 - protein, 18 aa, Artificial Sequence
  Description of Artificial Sequence Synthetic peptide
QIASGASAATTNVASAQC

SEQ ID NO: 92 - protein, 17 aa, Artificial Sequence
  Description of Artificial Sequence Synthetic peptide
QIASGASAATTNVASAQ

SEQ ID NO: 93 - protein, 16 aa, Artificial Sequence
  Description of Artificial Sequence Synthetic peptide
QIASGASAATTNVASA

SEQ ID NO: 94 - protein, 15 aa, Artificial Sequence
  Description of Artificial Sequence Synthetic peptide
QIASGASAATTNVAS

SEQ ID NO: 95 - protein, 14 aa, Artificial Sequence
  Description of Artificial Sequence Synthetic peptide
QIASGASAATTNVA

SEQ ID NO: 96 - protein, 13 aa, Artificial Sequence
  Description of Artificial Sequence Synthetic peptide
QIASGASAATTNV

SEQ ID NO: 97 - protein, 12 aa, Artificial Sequence
  Description of Artificial Sequence Synthetic peptide
QIASGASAATTN

SEQ ID NO: 98 - protein, 11 aa, Artificial Sequence
  Description of Artificial Sequence Synthetic peptide
QIASGASAATT

SEQ ID NO: 99 - protein, 10 aa, Artificial Sequence
  Description of Artificial Sequence Synthetic peptide
QIASGASAAT

SEQ ID NO: 100 - protein, 9 aa, Artificial Sequence
  Description of Artificial Sequence Synthetic peptide
QIASGASAA

SEQ ID NO: 101 - protein, 8 aa, Artificial Sequence
  Description of Artificial Sequence Synthetic peptide
QIASGASA

SEQ ID NO: 102 - protein, 8 aa, Artificial Sequence
  Description of Artificial Sequence Synthetic peptide
QIASGASA

SEQ ID NO: 103 - protein, 17 aa, Artificial Sequence
  Description of Artificial Sequence Synthetic peptide
IASGASAATTNVASAQC

SEQ ID NO: 104 - protein, 16 aa, Artificial Sequence
  Description of Artificial Sequence Synthetic peptide
IASGASAATTNVASAQ

SEQ ID NO: 105 - protein, 15 aa, Artificial Sequence
  Description of Artificial Sequence Synthetic peptide
IASGASAATTNVASA

SEQ ID NO: 106 - protein, 14 aa, Artificial Sequence
  Description of Artificial Sequence Synthetic peptide
IASGASAATTNVAS

SEQ ID NO: 107 - protein, 13 aa, Artificial Sequence
  Description of Artificial Sequence Synthetic peptide
IASGASAATTNVA

SEQ ID NO: 108 - protein, 12 aa, Artificial Sequence
  Description of Artificial Sequence Synthetic peptide
IASGASAATTNV

SEQ ID NO: 109 - protein, 11 aa, Artificial Sequence
  Description of Artificial Sequence Synthetic peptide
IASGASAATTN

SEQ ID NO: 110 - protein, 10 aa, Artificial Sequence
  Description of Artificial Sequence Synthetic peptide
IASGASAATT

SEQ ID NO: 111 - protein, 9 aa, Artificial Sequence
  Description of Artificial Sequence Synthetic peptide
IASGASAAT

SEQ ID NO: 112 - protein, 8 aa, Artificial Sequence
  Description of Artificial Sequence Synthetic peptide
IASGASAA

SEQ ID NO: 113 - protein, 7 aa, Artificial Sequence
  Description of Artificial Sequence Synthetic peptide
IASGASA

SEQ ID NO: 114 - protein, 6 aa, Artificial Sequence
  Description of Artificial Sequence Synthetic peptide
IASGAS

SEQ ID NO: 115 - protein, 16 aa, Artificial Sequence
  Description of Artificial Sequence Synthetic peptide
ASGASAATTNVASAQC

SEQ ID NO: 116 - protein, 15 aa, Artificial Sequence
  Description of Artificial Sequence Synthetic peptide
ASGASAATTNVASAQ

SEQ ID NO: 117 - protein, 14 aa, Artificial Sequence
  Description of Artificial Sequence Synthetic peptide
ASGASAATTNVASA

SEQ ID NO: 118 - protein, 13 aa, Artificial Sequence
  Description of Artificial Sequence Synthetic peptide
ASGASAATTNVAS

SEQ ID NO: 119 - protein, 12 aa, Artificial Sequence
  Description of Artificial Sequence Synthetic peptide
ASGASAATTNVA

SEQ ID NO: 120 - protein, 11 aa, Artificial Sequence
  Description of Artificial Sequence Synthetic peptide
ASGASAATTNV

SEQ ID NO: 121 - protein, 10 aa, Artificial Sequence
  Description of Artificial Sequence Synthetic peptide
ASGASAATTN

SEQ ID NO: 122 - protein, 9 aa, Artificial Sequence
  Description of Artificial Sequence Synthetic peptide
ASGASAATT

SEQ ID NO: 123 - protein, 8 aa, Artificial Sequence
  Description of Artificial Sequence Synthetic peptide
ASGASAAT

SEQ ID NO: 124 - protein, 7 aa, Artificial Sequence
  Description of Artificial Sequence Synthetic peptide
ASGASAA

SEQ ID NO: 125 - protein, 6 aa, Artificial Sequence
  Description of Artificial Sequence Synthetic peptide
ASGASA

SEQ ID NO: 126 - protein, 5 aa, Artificial Sequence
  Description of Artificial Sequence Synthetic peptide
ASGAS

SEQ ID NO: 127 - protein, 15 aa, Artificial Sequence
  Description of Artificial Sequence Synthetic peptide
SGASAATTNVASAQC

SEQ ID NO: 128 - protein, 14 aa, Artificial Sequence
  Description of Artificial Sequence Synthetic peptide
SGASAATTNVASAQ

SEQ ID NO: 129 - protein, 13 aa, Artificial Sequence
  Description of Artificial Sequence Synthetic peptide
SGASAATTNVASA

SEQ ID NO: 130 - protein, 12 aa, Artificial Sequence
  Description of Artificial Sequence Synthetic peptide
SGASAATTNVAS

SEQ ID NO: 131 - protein, 11 aa, Artificial Sequence
  Description of Artificial Sequence Synthetic peptide
SGASAATTNVA

SEQ ID NO: 132 - protein, 10 aa, Artificial Sequence
  Description of Artificial Sequence Synthetic peptide
SGASAATTNV

SEQ ID NO: 133 - protein, 9 aa, Artificial Sequence
  Description of Artificial Sequence Synthetic peptide
SGASAATTN

SEQ ID NO: 134 - protein, 8 aa, Artificial Sequence
  Description of Artificial Sequence Synthetic peptide
SGASAATT

SEQ ID NO: 135 - protein, 7 aa, Artificial Sequence
  Description of Artificial Sequence Synthetic peptide
SGASAAT

SEQ ID NO: 136 - protein, 6 aa, Artificial Sequence
  Description of Artificial Sequence Synthetic peptide
SGASAA

SEQ ID NO: 137 - protein, 5 aa, Artificial Sequence
  Description of Artificial Sequence Synthetic peptide
SGASA

SEQ ID NO: 138 - protein, 14 aa, Artificial Sequence
  Description of Artificial Sequence Synthetic peptide
GASAATTNVASAQC

SEQ ID NO: 139 - protein, 13 aa, Artificial Sequence
  Description of Artificial Sequence Synthetic peptide
GASAATTNVASAQ

SEQ ID NO: 140 - protein, 12 aa, Artificial Sequence
  Description of Artificial Sequence Synthetic peptide
GASAATTNVASA

SEQ ID NO: 141 - protein, 11 aa, Artificial Sequence
  Description of Artificial Sequence Synthetic peptide
GASAATTNVAS

SEQ ID NO: 142 - protein, 10 aa, Artificial Sequence
  Description of Artificial Sequence Synthetic peptide
GASAATTNVA

SEQ ID NO: 143 - protein, 9 aa, Artificial Sequence
  Description of Artificial Sequence Synthetic peptide
GASAATTNV

SEQ ID NO: 144 - protein, 8 aa, Artificial Sequence
  Description of Artificial Sequence Synthetic peptide
GASAATTN

SEQ ID NO: 145 - protein, 7 aa, Artificial Sequence
  Description of Artificial Sequence Synthetic peptide
GASAATT

SEQ ID NO: 146 - protein, 6 aa, Artificial Sequence
  Description of Artificial Sequence Synthetic peptide
GASAAT

SEQ ID NO: 147 - protein, 5 aa, Artificial Sequence
  Description of Artificial Sequence Synthetic peptide
GASAA

SEQ ID NO: 148 - protein, 13 aa, Artificial Sequence
  Description of Artificial Sequence Synthetic peptide
ASAATTNVASAQC

SEQ ID NO: 149 - protein, 12 aa, Artificial Sequence
  Description of Artificial Sequence Synthetic peptide
ASAATTNVASAQ

SEQ ID NO: 150 - protein, 11 aa, Artificial Sequence
  Description of Artificial Sequence Synthetic peptide
ASAATTNVASA

SEQ ID NO: 151 - protein, 10 aa, Artificial Sequence
  Description of Artificial Sequence Synthetic peptide
ASAATTNVAS

SEQ ID NO: 152 - protein, 9 aa, Artificial Sequence
  Description of Artificial Sequence Synthetic peptide
ASAATTNVA

SEQ ID NO: 153 - protein, 8 aa, Artificial Sequence
  Description of Artificial Sequence Synthetic peptide
ASAATTNV

SEQ ID NO: 154 - protein, 7 aa, Artificial Sequence
  Description of Artificial Sequence Synthetic peptide
ASAATTN

SEQ ID NO: 155 - protein, 6 aa, Artificial Sequence
  Description of Artificial Sequence Synthetic peptide
ASAATT

SEQ ID NO: 156 - protein, 5 aa, Artificial Sequence
  Description of Artificial Sequence Synthetic peptide
ASAAT

SEQ ID NO: 157 - protein, 12 aa, Artificial Sequence
  Description of Artificial Sequence Synthetic peptide
SAATTNVASAQC

SEQ ID NO: 158 - protein, 11 aa, Artificial Sequence
  Description of Artificial Sequence Synthetic peptide
SAATTNVASAQ

SEQ ID NO: 159 - protein, 10 aa, Artificial Sequence
  Description of Artificial Sequence Synthetic peptide
SAATTNVASA

SEQ ID NO: 160 - protein, 9 aa, Artificial Sequence
  Description of Artificial Sequence Synthetic peptide
SAATTNVAS

SEQ ID NO: 161 - protein, 8 aa, Artificial Sequence
  Description of Artificial Sequence Synthetic peptide
SAATTNVA

SEQ ID NO: 162 - protein, 7 aa, Artificial Sequence
  Description of Artificial Sequence Synthetic peptide
SAATTNV

SEQ ID NO: 163 - protein, 6 aa, Artificial Sequence
  Description of Artificial Sequence Synthetic peptide
SAATTN

SEQ ID NO: 164 - protein, 5 aa, Artificial Sequence
  Description of Artificial Sequence Synthetic peptide
SAATT

SEQ ID NO: 165 - protein, 543 aa, Artificial Sequence
  Description of Artificial Sequence Synthetic polypeptide
MNFLISKLKFYVLFIGIVCFCLTFILPNTSYFSSSLFKEIVVVLGFLILLTNQILSLKEIILPKKAPLLFILFLFLFLFLFFQYLFKLIISFQDLFFNLIYISVFFLSIIFGLNSKKYNQIILIHWILFSLIFSALISFLIGLNQKIRIIESPYLFGVSYNGRATANLGQPNQLSTLTLMAFFSLFYLKKYYKINKLFFYSIIISLIFCNVLTQSRSAWLSVILISIFFITKFPDKKNVLSVFCLNLVFWLSTILIPFFFNYFYPIGNSYTTLDRMKLSSSRFDIWPQLFLATFDKPFLGYGAGQVGLAQIESISNVSTRGEWFTYSHNIFLDFVIWYGWIVGSLVSFFIISLLIKISKSDLNRNETYLFVIILVFFFHCLLEYPYSYFYFLIPIGIISGFLLKLKSDDIFVLKKMYLCIVVFLSWLLFTLFTYQLIELGEKKESYSLQYLFKSSVKPIQSNLFILDGYSEKLDIEYLDYCYLIKNKDKEFFRRVAYRYPSTVSVSKYYSTQSDNLKNAENIVQAYQVISNRVYQPHIKKCNN

SEQ ID NO: 166 - protein, 548 aa, Artificial Sequence
  Description of Artificial Sequence Synthetic polypeptide
MNSIFKKIKNYTIVSGVFFLGSAFIIPNTSNLSSTLYKELIAVLGLLILLTVKSFDYKKILIPKNFYWFLFVIFIIFIQLIVGEIYFFQDFFFSISFLVILFLSFLLGFNERLNGDDLIVKKIAWIFIIVVQISFLIAINQKIEIVQNFFLFSSSYNGRSTANLGQPNQFSTLILITLFLLCYLREKNSLNNMVFNILSFCLIFANVMTQSRSAWISVILISLLYLLKFQKKIELRRVIFFNIVFWTLVYCVPLLFNLIFFQKNSYSTFDRLTMGSSRFEIWPQLLKAVFHKPFIGYGWGQTGVAQLETINKSSTKGEWFTYSHNLFLDLMLWNGFFIGLIISILILCFLIELYSSIKNKSDLFLFFCVVAFFVHCLLEYPFAYTYFLIPVGFLCGYISTQNIKNSISYFNLSKRKLTLFLGCCWLGYVAFWVEVLDISKKNEIYARQFLFSNHVKFYNIENYILDGFSKQLDFQYLDYCELKDKYQLLDFKKVAYRYPNASIVYKYYSISAEMKMDQKSANQIIRAYSVIKNQKIIKPKLKFCSIEY

SEQ ID NO: 167 - protein, 539 aa, Artificial Sequence
  Description of Artificial Sequence Synthetic polypeptide
MINILNKFKDCLIIIGLGCLCLAFFLPNTSNFSSSLFKEFFAVLGFLFILTVQFFFLKKIVVPSKLFILFILFIFLFIQYVFNLIINFQDLFFNLIYISIFFLSIIFGLNSKKYNNSVLIHWILFSLIFSALVSFLISLNQKIRIIESPYLFCVSYNGRATANLGQPNQLSTLTLMAFFSLFYLKKYYKINKLFFYTIIISLIFCNVLTQSRSAWLSVILISTFFITKFPDKKNVLSVFCLNLVFWLSTILIPFFFNYFYPIGNSYTTLDRMKLSSSRFDIWPQLFLATFDKPFLGYGAGQVGLAQIESISNASTRGEWFTYSHNIFLDFVIWYGWIVGSLVSFFIISLLIKISKSDLNRNKTYLFIIILVFFFHCLLEYPYSYFYFLIPIGIISGFLLKLKSDGVFVLKKIYLCIVIFLSWLLFALFTYQLIELDEKKESYSLQYLFKSSVKPIQSNLFILDGYSEKLDIEYLDYCYLIKNRDKEFFRRVAYRYPSTVSVSKYYSTQSDNLKNAENIVQAYQVISNRVYQPNIKKCNN

SEQ ID NO: 168 - protein, 525 aa, Artificial Sequence
  Description of Artificial Sequence Synthetic polypeptide
MRLYLSFLLLGLSYLSPNSSLLWPNSLQDFFAILSLILLLLTFNLNNFLINKYLFLVFLLLISIPVIQYNLKIIYFKQELFLSCLYITIFFSSIFLGSSIHNSQKVFIKFSIFFLVIGVLCVLIQIFQWIAVYSSIFINDLNSSRLSANIGQPNQLASLLSISLISCLILYKNKKIKVLIFSTCSVLIIFGIVLTQSRTSWLIFILIILFSYFKKNLKLTKYVTIFSTIFYGLLITYPFFYNSIHKKDISIIQRLNSDYSRLDIWQQMLFAIIERPWFGYGWNQTSVAQTEISLYHTTSIWIEYSHNLFLDFLIWNGIPLGIILITIIIFWFIYMYVNIKDLNSFMILIIISSFFIHCLLEFPFAYAYFIFPIGLYIGIINKRYLKYNYFNFNNWNYIFGLIIIFLLFFIVKDYIKITEKHKEYSLKYFSDNSILPNKLDIYLLDSLNVKEDIQYLDICYLIKIYNSEEIRNNFLRYPTNKSAVSLYYISLYNKNVSLETISFMKWKFQNLDLNTLKINKRCNTL

SEQ ID NO: 169 - protein, 539 aa, Artificial Sequence
  Description of Artificial Sequence Synthetic polypeptide
MSFYCYKYLNLFGIFLMGVAYFSTITLFFSTTFYKEIFAVAGLLFFLTALCFQYKIVSTQLLLFNLALLLIPMIQYAFGIIFFLQDALLSTVYLCIFLCSILVGVNFKANHQTNILNIFLAMLVFVGCISVLMAFNQRFMWFNSYLLFSSSYGNRATANLAQPNQLSTLLIMSLFSLFYLYQAQKIKKIIMYGITFILLIGIVMTQSRSAWASCIVLSALYLYYYHQKQDIINVIKLNVVFIGLTLCIPFLLNVLTYSQASTAIDRLQGGSTRFKIWPQLLHAVMEQPWTGYGWGQVDVAQLSTMTPTSTKKELFTYSHNLFLDLLLWNGLVLGTLLSLLIIYILYRCYMNLQYKQDLLLFLGFMAFFVHSCLEYPYAYTYFLIPAGMFLGYVSYQQNIKEVLIQINKKLYVIFLILLCIIFGCFLIEVNHLNEKSDLYARQNLFHEKVDFNDQKFYFLDGYSTSLDFQTIPYCNLVQYYPLITFKQIAYRYPSALTIAKYWLFSQKQQQMVRDAEQLRQAYVLLTKSGQHTFNNKVCN

SEQ ID NO: 170 - protein, 519 aa, Artificial Sequence
  Description of Artificial Sequence Synthetic polypeptide
MQLILIILGLSYLNPNSFLPWPNAMQDFCAMVALILLTATQFIKKNIQINKNTFYLFLFILSIPIIQFLFNILFFKQELFLSILYISIFFLSIIYGINQKEASNRIIKVSFFFVSVGIVCVFIQIIQWTNIYYSPFILESNYLRPSANLGQPNNLATLLFICLFSNLYIFKNKKINTSFYISINIFIIFGIALTQSRTSWIVFIALLILSHFKKELKLFKTIMINSILFFILVLITPYITLFYHGKGLTIIERINSDYSRLSIWKQIIIAITNKPLTGYGWNQTSVAQTQISLKYPIKVWLEYSHNMFLDILVWTGIPIGLLIITLINKWLFKTYQNIKNTNQLIIFFIIISFFIHCMFEFPFAYAYFLIPVGIYIGFLNKQDYNIITINIFTILLFLLISTLLTIITIDYMVLSEKRNNYSTKYLFSKKISPLESNIKILDALDLHNDILFLNDCYILKNKSIKNIKHIFYRYPTNKNIVIYYRFSLYYKNSSKEVIEYMKLKYPNFDSNQSKYNMCN

SEQ ID NO: 171 - protein, 521 aa, Artificial Sequence
  Description of Artificial Sequence Synthetic polypeptide
MPIFYFILGLSYLSPIFMQPWVSAFQDLCAIIAIILLMSIQSYRKNIEIDRRVLYVFGFIVCIPLVQYLFGILFFTQELVLSLIYISVFFLSIISGANFNRSYKNEEKLSFFFVFIGLSCVFIQLIQWSGLYHSALILDSSSRRPFANIGQPNNLATLLFIGFFSNILLFKNNRLKAKFYFLISAVLMTGIVLTQSRTSWLVFVSVLLLAFFKSKLELFSIMLKSSVLFFCLVLILPYITLFFHDQGLTVTERISSDSSRLYIWKQMLIAIMDKPWFGYGWNQTSVAQTSVTLKYPLDIWLEYSHNLFLDLIVWTGIPIGLSIIGIIIIWFLQTFKKINTLNQLLYFFIIAAFLIHCMLEYPFAYAYFLVPIGLYVGMLHQQLYETKNLKFKSLVITLVSILIITIIIISRDYFVLSDKRTIYTSESLFSEQVKPAFSKVLVLDALDVNNDILFLNRCYVLKKNTIENFKSNFYRYPTRMNLVMYYKSTIYYEKNSRDAERYMTAWYPDYKQNLSQYDICS

SEQ ID NO: 172 - protein, 523 aa, Artificial Sequence
  Description of Artificial Sequence Synthetic polypeptide
MLIFYIMLGLSYLSPNIFLPWLNALQDLFAIFALIILVSKQSYRKDIEIDERVIYVFGLIALIPLVQYLFGLLFFTQELVLSLIYISAFFLSIISGINLTKSFKEIEKISFSFIFISLSCVLLQLIQWSNIYHSALLLDSSSRRPFANIGQPNNLATLLFIGFFSNILLFKNNKIKIYLYLLVSATLMTGIVLTQSRTSWLVFIAVLFITFLKKKLNLFSTMLKSSIAFLFLVLTLPYITLFFHDQGLTVIERISSDSSRLYIWKQMLIAIIDKPWFGYGWNQTSVAQTSVTLKYPLNIWLEYSHNLFLDIIVWTGIPIGISIITIIIIWFLQTFKKINTPNQLIYFLIITAFFIHCMLEFPFAYAYFLLPVGLYVGILHQQVYETKNSKVKGLVMTIVTVLIVAVIIISRDYFLFNNKRTIYASKNLFSQQIQPISSKILLLNALDINNDILFLDECYVLKNNKFKVLRNSFYRYPTNKNLITYYKSAIYNNQNTQYPEKYMQKEYSNFKSSPAIYNNCSKL

SEQ ID NO: 173 - protein, 21 aa, Acinetobacter baylyi
CVGVQEISASNATTNVATATC

SEQ ID NO: 174 - protein, 23 aa, Acinetobacter soli
CTGVTQIASGASAATTNVASAQC

SEQ ID NO: 175 - protein, 21 aa, Acinetobacter soli
CVGVQEINASSSTSNVATATC

SEQ ID NO: 176 - protein, 21 aa, Acinetobacter sp.
CAGVETIGASNKTKNVESAAC

SEQ ID NO: 177 - protein, 21 aa, Acinetobacter radioresistens
CVGVQTIAASNATKNVATATC

SEQ ID NO: 178 - protein, 5 aa, Artificial Sequence
  Description of Artificial Sequence Synthetic peptide
  MOD_RES (1)..(1): D or E
  MOD_RES (2)..(2): A, C, D, E, F, G, H, I, K, L, M, N, Q, R, S, T, V, W or Y
  MOD_RES (3)..(3): Glycosylated Asparagine
  MOD_RES (4)..(4): A, C, D, E, F, G, H, I, K, L, M, N, Q, R, S, T, V, W or Y
  MOD_RES (5)..(5): S or T
XXNXX

SEQ ID NO: 179 - protein, 8 aa, Artificial Sequence
  Description of Artificial Sequence Synthetic peptide
  MOD_RES (6)..(6): Glycosylated Serine
WPAAASAP

SEQ ID NO: 180 - protein, 10 aa, Artificial Sequence
  Description of Artificial Sequence Synthetic peptide
DPRNVGGDLD

SEQ ID NO: 181 - protein, 7 aa, Artificial Sequence
  Description of Artificial Sequence Synthetic peptide
QPGKPPR

SEQ ID NO: 182 - protein, 4 aa, Artificial Sequence
  Description of Artificial Sequence Synthetic peptide
GGGS

SEQ ID NO: 183 - protein, 30 aa, Artificial Sequence
  Description of Artificial Sequence Synthetic polypeptide
GTSMPSSGNCTGVTQIASGASAATTNVASA

SEQ ID NO: 184 - protein, 30 aa, Artificial Sequence
  Description of Artificial Sequence Synthetic polypeptide
TSMPSSGNCTGVTQIASGASAATTNVASAQ

SEQ ID NO: 185 - protein, 30 aa, Artificial Sequence
  Description of Artificial Sequence Synthetic polypeptide
SMPSSGNCTGVTQIASGASAATTNVASAQC

SEQ ID NO: 186 - protein, 30 aa, Artificial Sequence
  Description of Artificial Sequence Synthetic polypeptide
MPSSGNCTGVTQIASGASAATTNVASAQCS

SEQ ID NO: 187 - protein, 30 aa, Artificial Sequence
  Description of Artificial Sequence Synthetic polypeptide
PSSGNCTGVTQIASGASAATTNVASAQCSD

SEQ ID NO: 188 - protein, 30 aa, Artificial Sequence
  Description of Artificial Sequence Synthetic polypeptide
SSGNCTGVTQIASGASAATTNVASAQCSDS

SEQ ID NO: 189 - protein, 30 aa, Artificial Sequence
  Description of Artificial Sequence Synthetic polypeptide
SGNCTGVTQIASGASAATTNVASAQCSDSD

SEQ ID NO: 190 - protein, 30 aa, Artificial Sequence
  Description of Artificial Sequence Synthetic polypeptide
GNCTGVTQIASGASAATTNVASAQCSDSDG

SEQ ID NO: 191 - protein, 30 aa, Artificial Sequence
  Description of Artificial Sequence Synthetic polypeptide
NCTGVTQIASGASAATTNVASAQCSDSDGV

SEQ ID NO: 192 - protein, 30 aa, Artificial Sequence
  Description of Artificial Sequence Synthetic polypeptide
CTGVTQIASGASAATTNVASAQCSDSDGVI

SEQ ID NO: 193 - protein, 30 aa, Artificial Sequence
  Description of Artificial Sequence Synthetic polypeptide
TGVTQIASGASAATTNVASAQCSDSDGVIT

SEQ ID NO: 194 - protein, 30 aa, Artificial Sequence
  Description of Artificial Sequence Synthetic polypeptide
GVTQIASGASAATTNVASAQCSDSDGVITV

SEQ ID NO: 195 - protein, 25 aa, Artificial Sequence
  Description of Artificial Sequence Synthetic peptide
GNCTGVTQIASGASAATTNVASAQC

SEQ ID NO: 196 - protein, 22 aa, Artificial Sequence
  Description of Artificial Sequence Synthetic peptide
  MOD_RES (2)..(2): V, T, A, or I
  MOD_RES (5)..(5): Q, T, E, A, or S
  MOD_RES (6)..(6): E, Q, T, or L
  MOD_RES (7)..(7): I or V
  MOD_RES (8)..(8): S, N, A, or G
  MOD_RES (9)..(9): S or absent
  MOD_RES (10)..(10): G, D, or absent
  MOD_RES (13)..(13): N, S, or A
  MOD_RES (14)..(14): A, S, or K
  MOD_RES (16)..(16): T, S, or K
  MOD_RES (19)..(19): A, E, Q, or L
  MOD_RES (20)..(20): T, S, or K
  MOD_RES (21)..(21): A or S
  MOD_RES (22)..(22): T, Q, A, or V
CXGVXXXXXXASXXTXNVXXXX

SEQ ID NO: 197 - protein, 22 aa, Artificial Sequence
  Description of Artificial Sequence Synthetic peptide
  MOD_RES (1)..(1): V, T, A, or I
  MOD_RES (4)..(4): Q, T, E, A, or S
  MOD_RES (5)..(5): E, Q, T, or L
  MOD_RES (6)..(6): I or V
  MOD_RES (7)..(7): S, N, A, or G
  MOD_RES (8)..(8): S or absent
  MOD_RES (9)..(9): G, D, or absent
  MOD_RES (12)..(12): N, S, or A
  MOD_RES (13)..(13): A, S, or K
  MOD_RES (15)..(15): T, S, or K
  MOD_RES (18)..(18): A, E, Q, or L
  MOD_RES (19)..(19): T, S, or K
  MOD_RES (20)..(20): A or S
  MOD_RES (21)..(21): T, Q, A, or V
XGVXXXXXXASXXTXNVXXXXC

SEQ ID NO: 198 - protein, 6 aa, Artificial Sequence
  Description of Artificial Sequence Synthetic 6xHis tag
HHHHHH

(19) United States
(12) Patent Application Publication
(10) Pub. No.: US 2024/0207383 A1
(43) Pub. Date: Jun. 27, 2024

(54) Minimal Sequons Sufficient for O-Linking Glycosylation

(71) Applicant: VaxNewMo LLC, St. Louis, MO (US)

(72) Inventors: Cory Knoot, St. Louis, MO (US); Christian Harding, St. Louis, MO (US)

(21) Appl. No.: 18/556,987

(22) PCT Filed: Apr. 27, 2022
(86) PCT No.: PCT/US2022/026517

§ 371 Date: Oct. 24, 2023

Related U.S. Application Data
(60) Provisional application No. 63/181,014, filed on Apr. 28, 2021.

(51) Int. Cl. A61K 39/09; A61K 47/54; A61K 47/64; A61P 37/04; A61K 39/00

(52) U.S. Cl. CPC A61K 39/092; A61K 47/549; A61K 47/646; A61P 37/04; A61K 2039/6037; A61K 2039/6075

(57) ABSTRACT

Provided herein are short ComP glycosylation fragments (sequons) and glycoconjugates containing ComP glycosylation fragments, and methods of making and using, for example, for use in the production of glycoconjugate vaccines.

### CROSS-REFERENCE TO RELATED APPLICATIONS

This PCT application claims the benefit of U.S. Provisional Appl. No. 63/181,014, filed Apr. 28, 2021.

This application is related to U.S. application Ser. No. 15/553,733, filed Aug. 25, 2017, which is a U.S. national stage application of PCT/CA2016/050208, filed Feb. 26, 2016, which claims the benefit of U.S. Provisional Appl. No. 62/121,439, filed on Feb. 26, 2015.

This application is also related to PCT/US2019/037251, filed Jun. 14, 2019, which claims the benefit of U.S. Provisional Appl. No. 62/685,970, filed on Jun. 16, 2018 and U.S. Provisional Appl. No. 62/783,971, filed on Dec. 21, 2018.

This application is also related to PCT/US2019/059893, filed Nov. 5, 2019, which claims the benefit of U.S. Provisional Appl. No. 62/783,971, filed on Dec. 21, 2018.

### STATEMENT REGARDING FEDERALLY-SPONSORED RESEARCH AND DEVELOPMENT

Statement under MPEP 310. This invention was made with the government support under the R44AI131742 grant awarded by the National Institute for Allergy and Infectious Disease (NIAID). The Government has certain rights in the invention.

### BACKGROUND

### Field of the Invention

This disclosure is directed to the field of glycosylation of proteins. In particular, glycosylation of and glycoconjugates containing very short glycosylation fragments of ComP. Also provided are methods of making, for example, for use in the production of glycoconjugate vaccines.

### Background Art

Protein glycosylation is the most common type of post-translational modification found in nature. Evidence for prokaryotic glycosylation was first reported in Campylobacter jejuni a little over two decades ago (Szymanski, C. M., et al., 1999) and functionally transferred into E. coli shortly thereafter (Wacker, M. et al., 2002)). Prokaryotic protein glycosylation is predominantly either O-linking or N-linking with O-linking systems attaching glycans to the side chains of serine or threonine residues and N-linking systems attaching glycans to asparagine side chains (Nothaft, H. & Szymanski, C. M., 2010; Schaffer, C. & Messner, 2017). Both O-linking and N-linking systems can be further grouped as oligosaccharyltransferase (OTase)-independent or OTase-dependent (Harding, C. M. & Feldman, 2019). OTase-independent glycosylation occurs in the cytoplasm and relies on dedicated glycosyltransferases to glycosylate cognate acceptor proteins. OTase-dependent glycosylation relies on an oligosacchryltransferase to transfer a preassembled oligosaccharide en bloc to acceptor proteins in the periplasm. The OTase-dependent protein glycosylation pathway shares many similarities to 0-antigen polysaccharide biosynthesis, starting with the transfer of a phosphorylated monosaccharide from a nucleotide-activated precursor to the lipid carrier undecaprenyl phosphate in the inner leaflet of the cytoplasmic membrane (Valvano, M. A., 2003; Hug, I. & Feldman, 2011). The lipid-linked monosaccharide is sequentially extended by the action of specific glycosyltransferases into a lipid-linked oligosaccharide, flipped to the periplasmic leaflet by a flippase (Raetz, C. R. & Whitfield, 2002) and subsequently transferred to an acceptor protein by an OTase. OTases are promiscuous and will transfer a variety of different glycans (Wacker, M. et al. 2006; Faridmoayer, A. et al. 2008), including long polysaccharides, from various bacterial species to acceptor proteins. This attractive property has led to the exploitation of OTases to transfer bacterial surface polysaccharides, like O-antigens and capsular polysaccharides (CPSs), to specific periplasmic carrier proteins, thereby generating polysaccharide-protein conjugates that are used as conjugate vaccines (Feldman, M. F. et al. 2005). This glycoengineering process is termed bioconjugation and, to date, three different OTases named PglB, PglL and PglS have been characterized and used for bioconjugate vaccine development.

PglB is a general N-linking OTase from C. jejuni and was the first bacterial OTase to be characterized and used in the production of glycoengineered bioconjugates in E. coli (Szymanski, C. M., et al. 1999; Feldman, M. F. et al. 2005). PglB naturally transfers polysaccharides that have a C2-acetamido sugar at the reducing end to acceptor proteins (Wacker, M. et al. 2006). While the natural glycan substrate versatility of PglB is the most restricted of all OTases, the N-linking sequon, D/E-X₁—N—X₂-S/T (SEQ ID NO: 178) where N is glycosylated and neither X₁ or X₂ are proline) (Kowarik, M. et al. 2006), is the shortest. The N-linking sequon of bacteria is similar to that recognized by Stt3, the catalytic subunit of the eukaryotic N-linking OTase complex (Kowarik, M. et al. 2006). PglL (also known as PglO) is a general 0-linking OTase first characterized from Neisseria species that transfers glycans with either a C2-acetamido sugar or galactose at the reducing end to acceptor proteins (Faridmoayer, A., Fentabil, et al., 2007). In contrast to PglB, there is no obvious conserved sequon for PglL, although glycosylation preferentially occurs in regions of low amino acid complexity rich in alanine, proline and glycine residues (Vik, A. et al. 2009). Recently, an optimized PglL sequon, WPAAASAP (SEQ ID NO: 179 where S is glycosylated), was derived from PilE, one of the natural pilin substrates for PglL (Pan, C. et al. 2016); however, the hydrophilic amino acid sequences DPRNVGGDLD (SEQ ID NO: 180) and QPGKPPR (SEQ ID NO: 181) were required to flank the optimized sequon in order for PglL to efficiently glycosylate proteins containing this tag. PglS, previously referred to PglLcomp, is an O-linking OTase that specifically glycosylates only one protein, ComP, a bacterial pilin protein of Acinetobacter species (Harding, C. M. et al., 2015). Importantly, PglS is the only known OTase capable of naturally transferring glycans with glucose at the reducing end in addition to glycans containing either galactose or a C2-acetamido sugar at the reducing end (Harding, C. M. et al., 2019). As such, PglS has the broadest polysaccharide substrate versatility of the three OTases employed for bioconjugate vaccine development.

In the last decade, PglB and to a lesser extent PglL, have been used to develop bioconjugate vaccines against Staphylococcus aureus, Shigella dysenteria, and flexneri, extraintestinal pathogenic E. coli, Salmonella species, and others (Wacker, M. et al., 2014; Hatz, C. F. et al. 2015; Huttner, A. et al., 2017; Sun, P. et al., 2018; van den Dobbelsteen, G. et al., 2016). However, the inability of PglB and PglL to naturally transfer polysaccharides with glucose at the reducing end prevents PglB and PglL from being used to make bioconjugate vaccines against several prominent bacterial threats (Harding, C. M. et al., 2019). For instance, ˜75% of Streptococcus pneumoniae (pneumococcus) capsules, >50% of Klebsiella pneumoniae capsules, and all ten Streptococcus agalactiae group B (GBS) capsules contain glucose as their reducing end sugar (Geno, K. A. et al., 2015; Pan, Y. J. et al., 2015; Berti, F. et al., 2014). The natural ability of PglS to transfer polysaccharides with glucose at the reducing end therefore lends itself well to the development of broad pneumococcal, K. pneumoniae and GBS vaccines that target the capsular polysaccharides of these pathogens. Indeed, using PglS and ComP as carrier protein or an engineered ComP fusion protein, the production of bioconjugate vaccines against current non-vaccine serotypes of pneumococcus as well as hypervirulent K. pneumoniae were reported (Harding, C. M. et al., 2019; Feldman, M. F. et al., 2019).

Although the polysaccharide substrate versatility of PglS makes it an attractive OTase for the production of next-generation bioconjugate vaccines, the minimal ComP sequon sufficient for PglS dependent glycosylation has not yet been identified. Previous bioconjugate vaccines developed using PglS have relied on using either the full-length native ComP protein, which is naturally a membrane-associated protein, as the carrier protein or an N-terminally truncated 117 amino acid ComP variant that was translationally fused at the C-terminus of the exotoxin A (EPA) from Pseudomonas aeruginosa. Identifying a shorter, more modular ComP sequon that is able to be efficiently glycosylated by PglS is preferable as the previous iterations containing the 117 amino acid ComP fragment is only amenable to glycosylation when it is translationally fused at the C-terminus of the carrier protein, limiting applications. With PglB for instance, knowledge of the short N-linking sequon has allowed multiple glycosylation sites to be engineered into the surface of carrier proteins, resulting in singly and multi-glycosylated bioconjugates (Ihssen, J. et al., 2010). It has also enabled more sophisticated in vitro studies involving different PglB peptide substrate variants and their effects on peptide binding and catalysis (Gerber, S. et al., 2013).

Thus, there remains a need to identify a short or minimal ComP sequon that could provide insights into the structural determinants of acceptor protein specificity in the PglS OTase family, facilitate comparisons to the sequons recognized by other O-linking OTases like PglL, and help guide improvements in glycoengineering design.

### SUMMARY

Provided for herein is a glycoconjugate comprising an oligo- or polysaccharide covalently linked to a fusion protein wherein the fusion protein comprises a ComP protein (ComP) glycosylation fragment and wherein the fusion protein is glycosylated with the oligo- or polysaccharide on the ComP glycosylation fragment at the serine residue corresponding to the conserved serine residue at position 82 of ComP110264 (SEQ ID NO: 1). In certain embodiments, the ComP glycosylation fragment does not contain a cysteine residue corresponding to the conserved cysteine residue at position 71 of ComP110264 (SEQ ID NO: 1) and/or does not contain a cysteine residue corresponding to the conserved cysteine residue at position 93 of ComP110264 (SEQ ID NO: 1). In certain embodiments, the ComP glycosylation fragment is located internally within the fusion protein. In certain embodiments, the ComP glycosylation fragment is solvent (or surface)-exposed. In certain embodiments, the ComP glycosylation fragment is integrated into a C10 β-turn, β-turn, β-twist, β-loop, U turn, reverse turn, chain reversal, or a hairpin loop of the fusion protein.

Provided for herein is a ComP glycosylation fragment comprising or consisting of an isolated fragment of a ComP protein wherein the ComP glycosylation fragment does not contain a cysteine residue corresponding to the conserved cysteine residue at position 71 of ComP₁₁₀₂₆₄ (SEQ ID NO: 1) and/or does not contain a cysteine residue corresponding to the conserved cysteine residue at position 93 of ComP₁₁₀₂₆₄ (SEQ ID NO: 1) and wherein the ComP glycosylation fragment comprises the serine residue corresponding to the conserved serine residue at position 82 of ComP₁₁₀₂₆₄ (SEQ ID NO: 1).

Provided for herein is a fusion protein comprising the ComP glycosylation fragment of this disclosure wherein the ComP glycosylation fragment is located internally within the fusion protein. In certain embodiments, the fusion protein is glycosylated by an oligo- or polysaccharide at a serine residue on the glycosylation fragment corresponding to the serine ComP glycosylation fragment residue at position 82 of SEQ ID NO: 1 (ComP₁₁₀₂₆₄).

Provided for herein is a method of in vivo conjugation of an oligo- or polysaccharide to an acceptor polypeptide, the method comprising covalently linking the oligo- or polysaccharide to the acceptor polypeptide with a PglS oligosaccharyltransferase (OTase), wherein the acceptor polypeptide comprises the ComP glycosylation fragment of this disclosure.

Provided for herein is a method of inducing a host immune response against a bacterial pathogen, the method comprising administering to a subject in need of the immune response an effective amount of the conjugate vaccine, the fusion protein, or the composition of this disclosure. Further provided for herein is a method of preventing or treating a bacterial disease and/or infection in a subject comprising administering to a subject in need thereof the conjugate vaccine, the fusion protein, or the composition of this disclosure.

Further provided for herein is a method of producing a pneumococcal conjugate vaccine against pneumococcal infection, the method comprising: (a) isolating the glycoconjugate or a glycosylated fusion protein of this disclosure; and (b) combining the isolated glycoconjugate or isolated glycosylated fusion protein with an adjuvant.

Further provided for is a glycoconjugate, glycosylated fusion protein, or conjugate vaccine for use in inducing a host immune response against a bacterial pathogen and/or preventing or treating a bacterial disease and/or infection in a subject.

### BRIEF DESCRIPTION OF THE DRAWINGS/FIGURES

FIG. 1A-E. FIG. 1A shows a schematic of EPA-ComP₁₁₀₂₆₄ fusion proteins where the ComP glycosylation fragment is fused at the C-terminus of the fusion protein. “ssDsbA” corresponds to the DsbA See secretion signal. GGGS (SEQ ID NO: 182) is a flexible linker between EPA and the ComP₁₁₀₂₆₄ fragment. FIG. 1B shows different amino acid sequences for ComP glycosylation fragments fused to C-terminus of the EPA fusion protein. The bold, underlined serine residue in each sequence corresponds to the conserved serine 82 of ComP₁₁₀₂₆₄ and is the site of glycosylation. The bold, underlined cysteine residues corresponding to Cys71 and Cys93 are also highlighted. (C2, SEQ ID NO: 183; D2; SEQ ID NO: 184; E2, SEQ ID NO: 185; F2, SEQ ID NO: 186; G2, SEQ ID NO: 187; H2, SEQ ID NO: 188; A3, SEQ ID NO: 189; B3, SEQ ID NO: 190; C3, SEQ ID NO: 191; D3, SEQ ID NO: 192; E3, SEQ ID NO: 193; F3, SEQ ID NO: 194; and C1, SEQ ID NO: 195).

FIG. 1C, FIG. 1D, and FIG. 1E show Western blot analysis of periplasmic extracts from E. coli SDB1 expressing PglS, the CPS8 glycan and an EPA-ComP₁₁₀₂₆₄ variant. Each lane of the Western blot panel corresponds to a strain of SDB1 expressing a different EPA-ComP variant with the ComP glycosylation fragment corresponding to the sequence shown in FIG. 1B. FIG. 1C shows proteins reacting with the anti-EPA antisera. FIG. 1D shows proteins reacting with the anti-His antisera. FIG. 1E shows the merged western blot images of FIG. 1C and FIG. 1D. Equivalent amounts of periplasmic extract based on OD₆₀₀ were loaded per lane. To the right of panels FIG. 1C-E, g₀ denotes unglycosylated EPA-ComP₁₁₀₂₆₄ and gₙ denotes EPA-ComP₁₁₀₂₆₄ glycosylated with different numbers of CPS8 repeat units. Protein mass markers (in kDa) are indicated to the left of panels FIG. 1C-E.

FIG. 2A-D. FIG. 2A shows a schematic of the CRM₁₉₇-ComP_{C1} fusion protein. “ssFlgI” corresponds to the FlgI SRP secretion signal. GGGS (SEQ ID NO: 182) is a flexible linker between CRM₁₉₇ and ComP_{C1}. FIG. 2B, FIG. 2C, and FIG. 2D show Western blot analysis of the purified CRM₁₉₇-ComP_{C1}-CPS8 glycoconjugate. FIG. 2B shows the proteins reacting with the anti-CPS8 antisera. FIG. 2C shows the proteins reacting with the anti-CRM₁₉₇ antisera. FIG. 2D shows the merged western blot images of FIG. 2B and FIG. 2C. Loss of CRM₁₉₇ and CPS8 signals in the proteinase K (PK)-treated samples demonstrate that the pneumococcal serotype 8 signal is CRM₁₉₇-linked and not the result of contamination from free polysaccharide or lipid-linked polysaccharide precursors. Protein mass markers (in kDa) are indicated to the left of panels FIG. 2B-D.

FIG. 3A,B. FIG. 3A shows schematic diagrams of the C- and N-terminal CRM₁₉₇ variants containing the C1 ComP glycosylation fragment. FIG. 3B shows Western blot analysis of periplasmic extracts of E. coli SDB1 expressing CRM₁₉₇-ComP_{C1} or ComP_{C1}-CRM₁₉₇ and the CPS8 glycan in the presence (+) or absence (−) of PglS. Equivalent amounts of periplasmic extracts based on OD₆₀₀ were loaded per lane. Protein mass markers (in kDa) are indicated to the left. GGGS (SEQ ID NO: 182).

FIG. 4A-E. FIG. 4A shows a schematic diagram of EPA fusion proteins containing ComP glycosylation fragments integrated internal of the EPA amino acid sequence. FIG. 4B shows amino acid sequences of the two iGT ComP glycosylation fragments inserted between EPA residues Ala489 and Arg489. These have either two terminal cysteines (“iG_{CC}”; SEQ ID NO: 30) or serines (“iG_{SS}”; SEQ ID NO: 31). FIG. 4C and FIG. 4D show Western blots on periplasmic extracts of E. coli SDB1 expressing the CPS8 glycan, EPA_{iGTcc} or EPA_{iGTss}, with (+) or without (−) PglS. FIG. 4C shows proteins reacting with the anti-EPA antisera. FIG. 4D shows proteins reacting with the anti-His antisera. FIG. 4E shows the merged Western blot images of FIG. 4C and FIG. 4D. Equivalent amounts of periplasmic extracts based on OD₆₀₀ were loaded per lane. Protein mass markers (in kDa) are indicated to the left of panels.

FIG. 5A-D. FIG. 5A show a schematic Diagram of EPA constructs containing ComP glycosylation fragments used for these experiments (from top to bottom, SEQ ID NOs: 6-28). Twenty-two to five amino acid-truncated variants of the iGT_{CC} ComP glycosylation fragment were inserted into the EPA coding sequence between Ala489 and Arg489. FIG. 5B shows the amino acid sequences of the 22 truncated iGT ComP glycosylation fragments with name designations assigned to the left. The underlined, bolded serine is the glycosylation site. (iGTcc SEQ ID NO: 30; Δ0-1 SEQ ID NO: 32; Δ1-0 SEQ ID NO: 43; Δ1-2 SEQ ID NO: 45; Δ2-1 SEQ ID NO: 56; Δ2-3 SEQ ID NO: 58; Δ3-2 SEQ ID NO: 69; Δ3-4 SEQ ID NO: 71; Δ4-3 SEQ ID NO: 82; Δ4-5 SEQ ID NO: 84; Δ5-4 SEQ ID NO: 95; Δ5-6 SEQ ID NO: 97; Δ6-5 SEQ ID NO: 108; Δ6-6 SEQ ID NO: 109; Δ6-7 SEQ ID NO: 110; Δ7-6 SEQ ID NO: 121; Δ7-7 SEQ ID NO: 122; Δ7-8 SEQ ID NO: 123; Δ8-7 SEQ ID NO: 134; Δ8-8 SEQ ID NO: 135; Δ8-9 SEQ ID NO: 136; Δ9-8 SEQ ID NO: 146; Δ9-9 SEQ ID NO: 147). FIG. 5C shows Western blot analysis on periplasmic extracts of E. coli SDB1 expressing PglS, CPS8 and an EPA_{iGT} fusion protein containing a truncated ComP glycosylation fragment. Each lane of the Western blot panel corresponds to a strain of SDB1 expressing a different EPA_{iGT} fusion protein containing a truncated ComP glycosylation fragment with the ComP glycosylation fragment corresponding to the sequence shown in FIG. 5B. FIG. 5C shows proteins reacting with the anti-EPA antisera probing with an anti-EPA antibody. EPA_{iGTcc} is shown for comparison. The “EPA” lane corresponds to EPA lacking any ComP-derived sequences and serves as a negative control. Equivalent amounts of periplasmic extract based on OD₆₀₀ were loaded per lane. FIG. 5D shows the same Western blot as above with an increase anti-EPA signal brightness in order to show low-level glycosylation for the smallest ComP glycosylation fragments.

FIG. 6A,B,C. FIG. 6 shows Western blot analysis of Ni affinity chromatography purified EPA fusion proteins containing the iGTΔ6-6 ComP glycosylation fragment integrated between residues Ala489 and Arg490 of EPA. The fusion protein was purified from SDB1 cells expressing the CPS8 glycan in the presence (+) or absence (−) of PglS. FIG. 6A shows proteins reacting with anti-His antisera. FIG. 6B shows proteins reacting with anti-CPS8 antisera. FIG. 6C shows a merge of FIG. 6A and FIG. 6B. Protein mass markers (in kDa) are indicated to the left of panels FIG. 6A-C.

FIG. 7A,B. FIG. 7A shows a schematic diagram of the EPA fusion protein containing the iGTΔ3-4 ComP glycosylation fragment integrated between residues Glu548 and Gly549 of EPA. The iGTΔ3-4 amino acid sequence is listed below the schematic (SEQ ID NO: 71). FIG. 7B shows Western blot analysis on periplasmic extracts of E. coli SDB1 expressing PglS, CPS8 and the EPA fusion protein containing the iGTΔ3-4 ComP glycosylation fragment integrated between residues Glu548 and Gly549. Protein reacting with the anti-EPA antisera probing with an anti-EPA antibody are shown.

FIG. 8A,B,C. FIG. 8 shows Western blot analysis of Ni affinity chromatography purified EPA fusion proteins containing the iGTΔ3-4 ComP glycosylation fragment integrated between residues Glu548 and Gly549 of EPA. The fusion protein was purified from SDB cells expressing the CPS8 glycan in the presence (+) or absence (−) of PglS. FIG. 8A shows proteins reacting with anti-His antisera. FIG. 8B shows proteins reacting with anti-CPS8 antisera. FIG. 8C shows a merge of FIG. 8A and FIG. 8B. Protein mass markers (in kDa) are indicated to the left of panels Figure A-C.

FIG. 9. FIG. 9 lists ComP ortholog amino acid sequences. The site of predicted glycosylation is bolded.

FIG. 10. FIG. 10 lists ComP Δ28 ortholog amino acid sequences in which the amino acids corresponding to the 28 N-terminal amino acids of ComP_{ADP1}: AAC45886.1 have been removed. The site of predicted glycosylation is bolded.

FIG. 11. FIG. 11 shows an alignment of a region ComP sequences including the serine (S) residue (boxed) corresponding to the serine residue at position 82 of ComP₁₁₀₂₆₄ (SEQ ID NO: 1) also corresponding to the serine residue at position 84 of ComP_{ADP1} (SEQ ID NO: 2).

### DETAILED DESCRIPTION

To the extent necessary to provide descriptive support, the subject matter and/or text of the appended claims is incorporated herein by reference in their entirety.

It will be understood by all readers of this written description that the exemplary aspects and embodiments described and claimed herein may be suitably practiced in the absence of any recited feature, element or step that is, or is not, specifically disclosed herein.

### Definitions

It is to be noted that the term “a” or “an” entity refers to one or more of that entity; for example, “a polysaccharide,” is understood to represent one or more polysaccharides. As such, the terms “a” (or “an”), “one or more,” and “at least one” can be used interchangeably herein.

Furthermore, “and/or” where used herein is to be taken as specific disclosure of each of the specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; Δ, B, or C; A or C; A or B; B or C; A and C; A and B; B and C; A (alone); B (alone); and C (alone).

It is understood that wherever aspects are described herein with the language “comprising” or “comprises” otherwise analogous aspects described in terms of “consisting of,” “consists of,” “consisting essentially of,” and/or “consists essentially of,” and the like are also provided.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, e.g., 1, 2, 3, or 4, unless otherwise stated, the disclosure specifically includes any range in between the values, e.g., 1 to 3, 1 to 4, 2 to 4, etc.

The headings provided herein are solely for ease of reference and are not limitations of the various aspects or aspects of the disclosure, which can be had by reference to the specification as a whole.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Disclosed herein are certain binding molecules, or antigen-binding fragments, variants, or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that cans specifically bind a given antigenic determinant, or epitope. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

The terms “antibody” and “immunoglobulin” can be used interchangeably herein. An antibody (or a fragment, variant, or derivative thereof as disclosed herein comprises at least the variable domain of a heavy chain and at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring,” or that are, or that might be at any time, determined or interpreted by a judge or an administrative or judicial body to be, “naturally-occurring.”

In certain embodiments, the polynucleotide or nucleic acid is DNA. In other embodiments, a polynucleotide can be RNA.

A “vector” is nucleic acid molecule as introduced into a host cell, thereby producing a transformed host cell. A vector can include nucleic acid sequences that permit it to replicate in a host cell, such as an origin of replication. A vector can also include one or more selectable marker gene and other genetic elements known in the art.

A “transformed” cell, or a “host” cell, is a cell into which a nucleic acid molecule has been introduced by molecular biology techniques. As used herein, the term transformation encompasses those techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the terms “treat,” “treatment,” or “treatment of” (e.g., in the phrase “treating a subject”) refers to reducing the potential for disease pathology, reducing the occurrence of disease symptoms, e.g., to an extent that the subject has a longer survival rate or reduced discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals, including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective, and that contains no additional components that are unacceptably toxic to a subject to which the composition would be administered. Such composition can be sterile.

An “effective amount” of an antibody as disclosed herein is an amount sufficient to carry out a specifically stated purpose. An “effective amount” can be determined empirically and in a routine manner, in relation to the stated purpose.

As used herein, a “sequon” refers to a specific sequence of amino acids consisting of amino acid residues for recognition and subsequent glycosylation by a specific oligosaccharyltransferase.

As used herein, a “glycoconjugate” refers to a polypeptide that is covalently linked to a carbohydrate moiety. It is understood that the carbohydrate moiety can be a monosaccharide, oligosaccharide, or polysaccharide. For purposes of this disclosure, a “glycoconjugate” is a specific type of “bioconjugate” as referred to herein.

### Overview

Conjugate vaccines, consisting of a polysaccharide linked to a protein, are lifesaving prophylactics. Traditionally, conjugate vaccines are manufactured using chemical methodologies. However, in vivo bacterial conjugations have emerged as manufacturing alternatives. In vivo conjugation (bioconjugation) is reliant upon an oligosaccharyltransferase to attach polysaccharides to proteins. Currently, the oligosaccharyltransferases employed for bioconjugations are not suitable for the generation of conjugate vaccines when the polysaccharides contain glucose at the reducing end. This limitation has enormous implications as ˜75% of Streptococcus pneumoniae capsules contain glucose as the reducing end sugar. Disclosed herein is the use of an O-linked oligosaccharyltransferase to generate the first ever polyvalent pneumococcal bioconjugate vaccine with polysaccharides containing glucose at their reducing end. Pneumococcal bioconjugates were immunogenic, protective, and rapidly produced with recombinant techniques. Certain aspects disclosed herein provide for the engineering, characterization, and immunological responses of a polyvalent pneumococcal bioconjugate vaccine using the natural acceptor protein ComP as a vaccine carrier as well as a monovalent pneumococcal bioconjugate vaccine using a conventional vaccine carrier; e.g., in certain aspects, containing the Pseudomonas aeruginosa exotoxin A protein. This establishes a platform to overcome limitations of other conjugating enzymes enabling the development of bioconjugate vaccines for many important human and animal pathogens.

Even with the introduction and implementation of pneumococcal conjugate vaccines over the last two decades, ˜1.5 million deaths are still attributed to S. pneumoniae each year. This is due in part to the 90+ serotypes of S. pneumoniae and the complex manufacturing methods required to synthesize pneumococcal conjugate vaccines. Together these factors hinder global distribution and development of broader, more protective variations of the vaccines. To expedite development and lower manufacturing costs, disclosed herein is a platform for developing conjugate vaccines, for example pneumococcal conjugate vaccines, using in vivo conjugation. This streamlined process has the potential to complement existing manufacturing pipelines or completely bypass the dependency on chemical conjugation methodologies, enabling the production of a more comprehensive conjugate vaccines.

Traditional, chemical conjugate vaccine synthesis is considered complex, costly, and laborious (Frasch, C. E. Vaccine 27, 6468-6470 (2009)) however, in vivo conjugation has been thoroughly progressing as a viable biosynthetic alternative (Huttner, A. et al. Lancet Infect Dis 17, 528-537 (2017)). These strides are best highlighted by the successes of GlycoVaxyn, (now LimmaTech Biologics AG an independent company with direct ties to GlaxoSmithKline), a clinical stage biopharmaceutical company with multiple bioconjugate vaccines in various phases of clinical trials, one of which (Flexyn2a) has just completed a Phase 2b challenge study. Although GlycoVaxyn has been at the forefront of the in vivo conjugation revolution, the ability to glycosylate carrier/acceptor proteins with polysaccharides containing glucose (Glc) as the reducing end sugar has been elusive and, expectedly, has stymied the development of a pneumococcal bioconjugate vaccine.

The oligosaccharyltransferase PglS—previously referred to as PglL by Schulz et al. (PMID 23658772) and PglLcomP by Harding et al. 2015 (PMID 26727908)—was only recently characterized as a functional OTase (Schulz, B. L. et al. PLoS One 8, e62768 (2013)). Subsequent mass spectrometry studies on total glycopeptides demonstrated that PglS does not act as a general PglL-like OTase, glycosylating multiple periplasmic and outer membrane proteins (Harding, C. M. et al. Mol Microbiol 96, 1023-1041 (2015)). In fact, the genome of A. baylyi ADP1 encodes for two OTase, a PglL-like ortholog (UniProtKB/Swiss-Prot: Q6FFS6.1), which acts as the general OTase and PglS (UniProtKB/Swiss-Prot: Q6F7F9.1), which glycosylates a single protein, ComP (Harding, C. M. et al. Mol Microbiol 96, 1023-1041 (2015)).

ComP is orthologous to type IV pilin proteins, like PilA from Pseudomonas aeruginosa and PilE from Neisseria meningiditis, both of which are glycosylated by the OTases TfpO (Castric, P. Microbiology 141 (Pt 5), 1247-1254 (1995)) and PglL (Power, P. M. et al. Mol Microbiol 49, 833-847 (2003)), respectively. Although TfpO and PglL also glycosylate their cognate pilins at serine residues, the sites of glycosylation differ between each system. TfpO glycosylates its cognate pilin at a C-terminal serine residue (Comer, J. E., Marshall, M. A., Blanch, V. J., Deal, C. D. & Castric, P. Infect Immun 70, 2837-2845 (2002)), which is not present in ComP. PglL glycosylates PilE at an internal serine located at position 63 (Stimson, E. et al. Mol Microbiol 17, 1201-1214 (1995)). ComP also contains serine residues near position 63 and the surrounding residues show moderate conservation to PilE from N. meningiditis. Comprehensive glycopeptide analysis, however, revealed this serine and the surrounding residues were not the site of glycosylation in ComP. PglS glycosylates ComP at a single serine residue located at position corresponding to the conserved serine at position 82 of ComP₁₁₀₂₆₄: ENV58402.1 (SEQ ID NO: 1) (also corresponding to the conserved serine at position 84 of ComP_{ADP1}: AAC4588631 (SEQ ID NO: 2)), which is a novel glycosylation site not previously found within the type IV pilin superfamily. The ability of PglS to transfer polysaccharides containing glucose as the reducing end sugar coupled with the identification of a novel site of glycosylation within the pilin superfamilies demonstrates that PglS is a functionally distinct OTase from PglL and TfpO.

### Bioinformatic Features of ComP Pilin Orthologs.

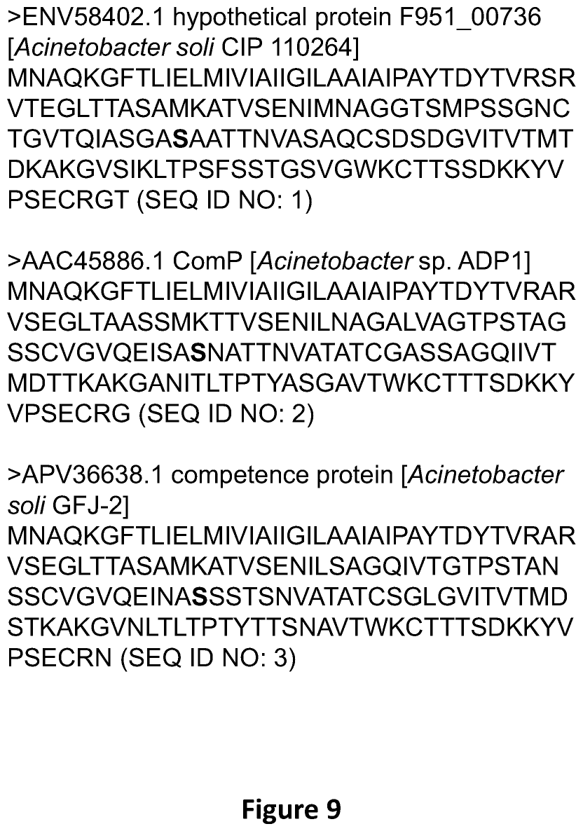

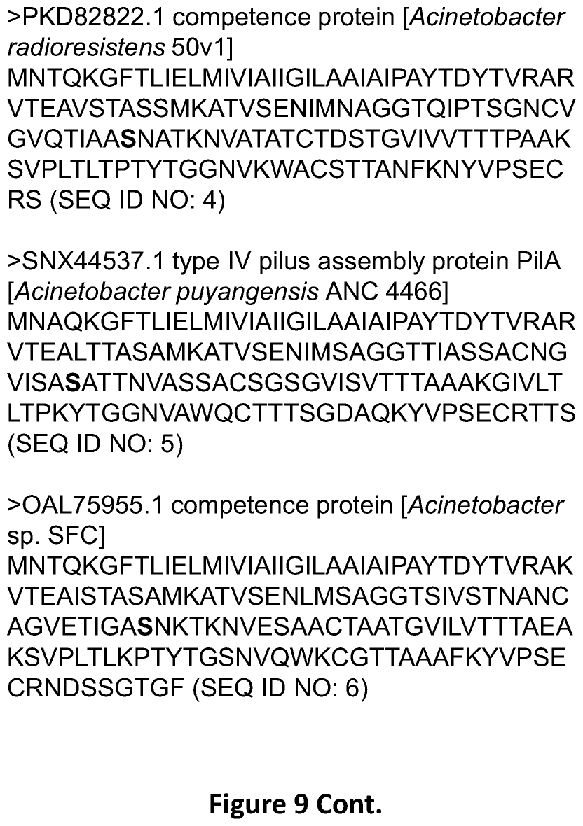

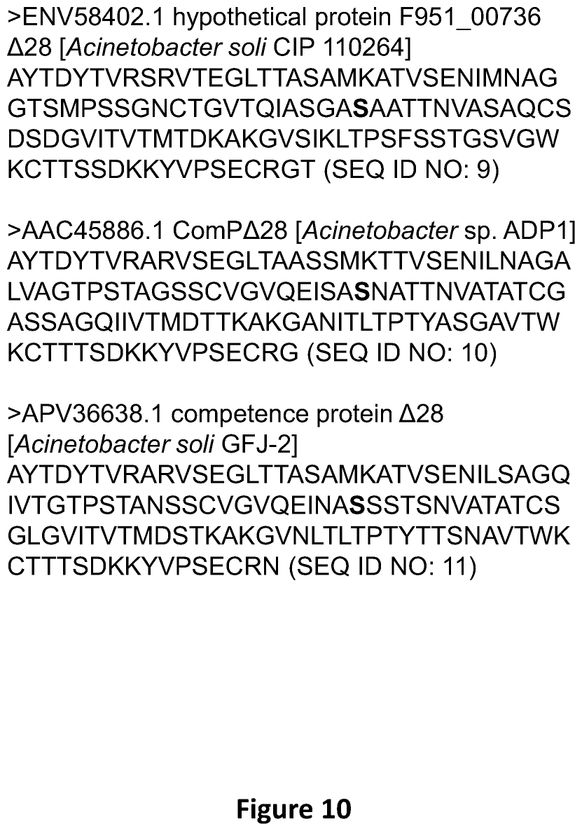

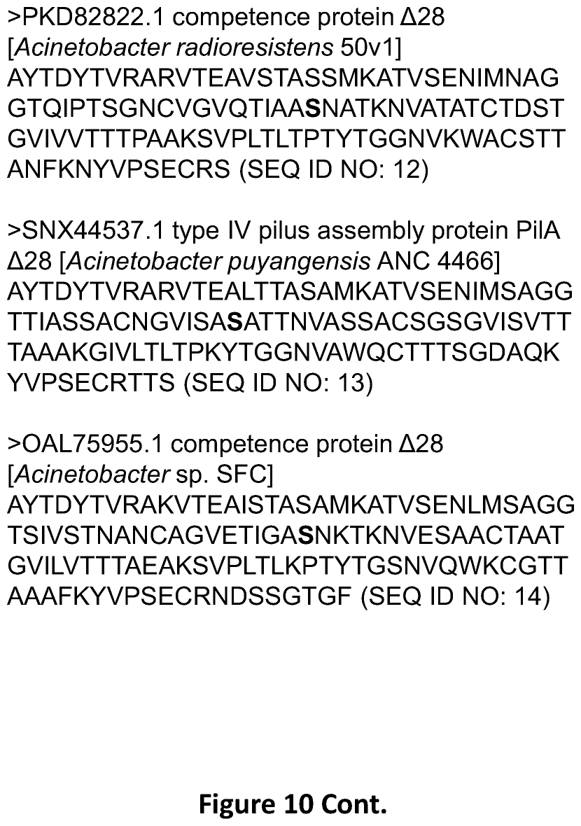

ComP was first described as a factor required for natural transformation in Acinetobacter baylyi ADP1 (Porstendorfer, D., Drotschmann, U. & Averhoff, B. Appl Environ Microbiol 63, 4150-4157 (1997)). In a subsequent study, it was demonstrated that ComP from A. baylyi ADP1 (herein referred to as ComPADP1) was glycosylated by a novel OTase, PglS, located immediately downstream of ComP, and not the general OTase PglL located elsewhere on the chromosome (Harding, C. M. et al. Mol Microbiol 96, 1023-1041 (2015)). The ComP_{ADP1} protein (NCBI identifier AAC45886.1) belongs to a family of proteins called type IV pilins. Specifically, ComP shares homology to type IVa major pilins (Giltner, C. L., Nguyen, Y. & Burrows, L. L. Microbiol Mol Biol Rev 76, 740-772 (2012)). Type IVa pilins share high sequence homology at their N-terminus, which encode for the highly conserved leader sequence and N-terminal alpha helix; however, the C-terminus display remarkable divergences across genera and even within species (Giltner, C. L., Nguyen, Y. & Burrows, L. L. Microbiol Mol Biol Rev 76, 740-772 (2012)). To help differentiate ComP orthologs from other type IVa pilin proteins, such as, PilA from A. baumannii, P. aeruginosa, and Haemophilus influenzae as well as PilE from Neisseria species (Pelicic, V. Mol Microbiol 68, 827-837 (2008)), a BLASTp analysis was performed comparing the primary amino acid sequence of ComP_{ADP1} against all proteins from bacteria in the Acinetobacter genus. Expectedly, many Acinetobacter type IVa pilin orthologs, including ComPADP1, share high homology at their N-termini; however, very few proteins display high sequence conservation across the entire amino acid sequence of ComP. At least six ComP orthologs (FIG. 9) were identified based on the presence of the conserved serine at position 84 relative to ComPADP1 as well as a conserved disulfide bond flanking the site of predicted glycosylation connecting the predicted alpha beta loop to the beta strand region (Giltner, C. L., Nguyen, Y. & Burrows, L. L. Microbiol Mol Biol Rev 76, 740-772 (2012)). Furthermore, all six ComP orthologs carry both a pglS homolog immediately downstream of the comP gene as well as a pglL homolog located elsewhere in the chromosome. Together, at least the presence of the conserved serine at position 84, the disulfide loop flanking the site of glycosylation, the presence of a pglS gene immediately downstream of comP, and the presence of a pglL homolog located elsewhere on the chromosome differentiate ComP pilin variants from other type IVa pilin variants.

Therefore, features common to ComP proteins are disclosed herein that identify ComP orthologs in different Acinetobacter species. ComP proteins can be differentiated from other pilins by the presence of the conserved glycosylated serine located at position 84 relative to the ADP1 ComP protein and the presence of a disulfide loop flanking the site of glycosylation. In addition, the presence of a pglS homolog immediately downstream of ComP is an indicator of ComP. Further to be classified as a PglS OTase protein rather than a PglL OTase protein, the OTase downstream of ComP must display higher sequence conservation with PglS (ACIAD3337) when compared to PglL (ACIAD0103) in A. baylyi ADP1. It is also evident to one of ordinary skill in the art that in any embodiment disclosed herein, a ComP protein comprises and is capable of being glycosylated on a serine residue corresponding to the conserved serine residue at position 82 of SEQ ID NO: 1 (ComP₁₁₀₂₆₄: ENV58402.1).

### ComP Protein Glycosylation Fragments.

It was previously demonstrated that the PglS ortholog from Acinetobacter baylyi strain ADP1 glycosylates the ComP ortholog from A. soli strain CIP 110264 at a single serine residue located at position 82 (Harding, C. M. et al., 2019; WO/2019/241672, which is incorporated by reference herein in its entirety). PglS was engineered to functionally glycosylate heterologous proteins by translationally fusing a large fragment (117 amino acids) of ComP to the C-terminus of a known carrier protein. Specifically, the 117 amino acid ComP₁₁₀₂₆₄ fragment was fused at the C-terminus of a genetically deactivated exotoxin A from Pseudomonas aeruginosa (EPA) between a flexible GGGS linker (SEQ ID NO: 182). This chimeric carrier protein also had an N-terminal DsbA signal sequence (ssDsbA) for translocation to the periplasm via the Sec-pathway as well as a C-terminal hexahistidine tag for detection.

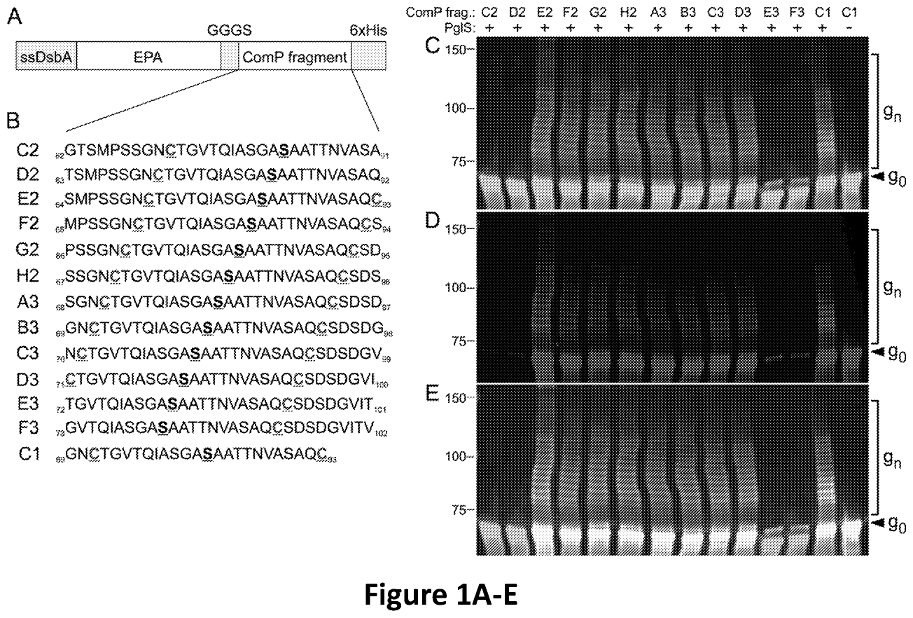

Even shorter ComP glycosylation fragments sufficient for glycosylation by PglS have been identified (WO/2020/131236, which is incorporated by reference herein in its entirety). It has been shown that ComP₁₁₀₂₆₄ glycosylation fragments fused to the C-terminus of the EPA carrier protein could also be glycosylated by PglS, but only if the ComP glycosylation fragments contained both cysteine residues corresponding to Cys71 and Cys93 relative to ComP₁₁₀₂₆₄. These observations were confirmed in a series of experiments aimed at identifying even shorter ComP glycosylation fragments. FIG. 1A and FIG. 1B show ComP₁₁₀₂₆₄ fragments that were designed to shift one amino acid N- to C-terminal relative to serine 82, which is the site of PglS glycosylation when the ComP glycosylation fragment was fused to the extreme C-terminus of the EPA carrier protein. The ComP glycosylation fragments were PCR amplified, cloned onto the C-terminus of EPA, and tested for bioconjugation by PglS. For these and all experiments described below, the serotype 8 pneumococcal capsular polysaccharide (CPS8) expressed from the pB-8 plasmid as the glycan source (Kay, E. J., et al., 2016) was used. The CPS8 glycan was selected as it contains glucose as the reducing end sugar and was previously demonstrated to be efficiently transferred to ComP by PglS (Harding, C. M. et al., 2019). In addition, for these and all experiments described below, bioconjugation was performed in the E. coli strain, SDB1. SDB1 has deletions of WecA, which initiates biosynthesis of the enterobacterial common antigen and the O-antigen polysaccharides, and WaaL, which transfers undecaprenyl-pyrophosphate linked glycan precursors to the outer core of lipid-A (Garcia-Quintanilla, F., et al., 2014). Collectively, these mutations facilitate the accumulation of heterologously expressed lipid-linked glycan precursors, like the CPS8 polysaccharide lipid-linked precursor, for exclusive use by PglS. SDB1 strains expressing the CPS8 glycan, PglS, and a fusion EPA-ComP₁₁₀₂₆₄ construct from IPTG inducible vectors were cultured in LB broth, induced at mid-log and grown overnight. Samples were harvested ˜20 hours after induction for western blot analysis on periplasmic extracts to assess EPA-ComP₁₁₀₂₆₄ fusion protein expression and protein glycosylation. Western blots were probed using antibodies against EPA (anti-EPA) and the hexahistidine tag (anti-His). Probing with both antibodies allowed ascertainment whether the EPA protein and/or the C-terminal ComP fragment remained intact.

FIG. 1C, FIG. 1D, and FIG. 1E reaffirm that the presence of Cys71 and Cys93 residues flanking Ser82 in ComP₁₁₀₂₆₄ are essential for EPA-ComP₁₁₀₂₆₄ glycosylation when the ComP glycosylation fragment is fused at the C-terminus. As seen in FIG. 1C, FIG. 1D, and FIG. 1E, fusion proteins containing ComP glycosylation fragments that lacked either Cys71 or Cys93 were not glycosylated. Only in fusion proteins containing ComP glycosylation fragments with both cysteine residues was transfer of the CPS8 glycan observed. The glycosylation efficiency and average number of the CPS8 repeat units transferred by PglS were similar for all fusion proteins containing ComP glycosylation fragments containing both Cys71 and Cys93. Upon closer examination of the Western blots, it was observed that chimeric EPA-ComP₁₁₀₂₆₄ variants (listed as C2, D2, E3, and F3 in FIG. 1C, FIG. 1D, and FIG. 1E) barely reacted with the anti-His antibody when compared to the anti-EPA signal (FIG. 1D). Furthermore, the anti-EPA channel revealed that these variants migrated with a slightly lower molecular weight when compared to the unglycosylated EPA-ComP₁₁₀₂₆₄ variants containing both Cys71 and Cys93 (FIG. 1C). Taken together, these observations indicated that the ComP fragments lacking both cysteine residues are unstable and likely prone to C-terminal degradation, thereby preventing glycosylation by PglS. Without being bound by theory, it is believed that Cys71 and Cys93 are able to stabilize ComP₁₁₀₂₆₄ by forming a covalent disulfide bridge.

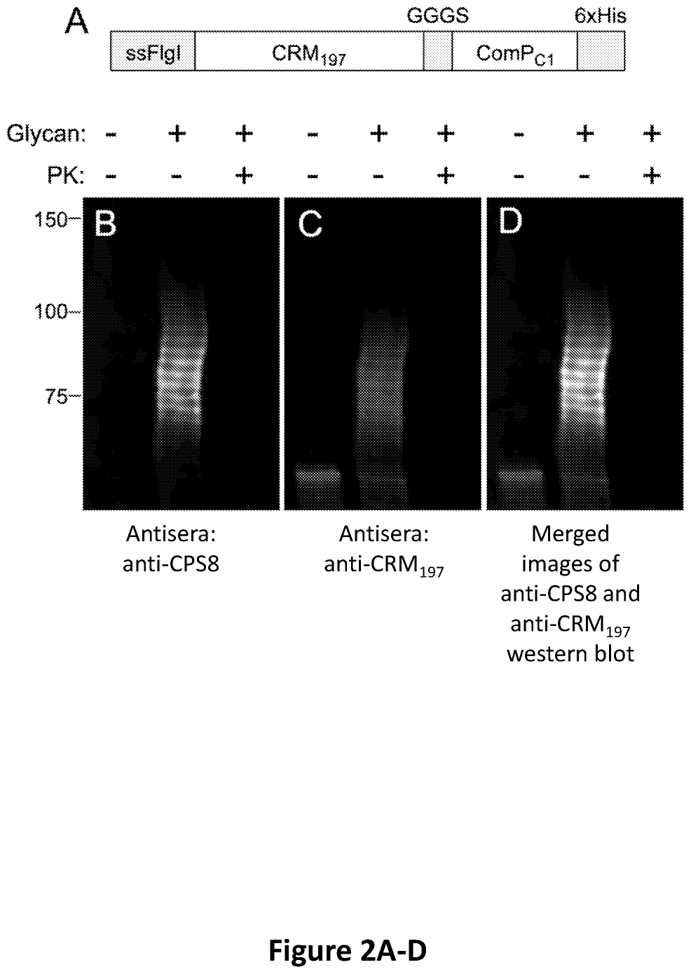

A variety of proteins from different organisms, typically inactivated bacterial toxins, have been used as carriers for conjugate and bioconjugate vaccines. Cross-reactive material 197 (CRM₁₉₇) is a genetically deactivated form of the diphtheria toxin that has been used extensively as the carrier protein in multiple conjugate vaccines for pneumococcus, Neisseria meningitidis, and Haemophilus influenza type b (Berti, F. & Adamo, R., 2018). Given the frequent use of CRM₁₉₇ in conjugate vaccine formulations the PglS bioconjugation system was extended to function with CRM₁₉₇. For these experiments, the 25-amino acid “C1” ComP glycosylation fragment (ComP_{C1}) previously identified was translationally fused to the C-terminus of CRM₁₉₇ linked by a GGGS sequence (SEQ ID NO: 182). An SRP-dependent FlgI secretion sequence (ssFlgI) was added to the N-terminus for CRM₁₉₇ for export to the periplasm (Goffin, P., et al., 2017). Finally, a C-terminal hexahistidine tag was added to aid purification (FIG. 2A). E. coli SDB1 cells expressing the CPS8 glycan along with PglS and the CRM₁₉₇-ComP_{C1} carrier (expected size of 61.8 kDa) were cultured in shake flasks and harvested after 24 hours. The CRM₁₉₇-ComP_{C1}-CPS8 glycoconjugate was purified with three successive rounds of chromatography. First, nickel-affinity chromatography was employed as the glycoconjugates contain a C-terminal hexahistidine tag. Fractions containing glycoconjugates were pooled and enriched for glycosylated glycoconjugates using a MonoQ column and eluted with a linear salt gradient. A final polishing step to remove large aggregates was performed on a Superdex 200 Increase column. As seen in FIG. 2B, FIG. 2C, and FIG. 2D, Western blotting on the purified samples using anti-CRM₁₉₇ and pneumococcal CPS8 antisera demonstrated that the CRM₁₉₇-ComP_{C1} carrier was glycosylated with CPS8. Digestion of the purified glycoconjugates with Proteinase K prior to separation on SDS-PAGE resulted in a complete loss of the CRM₁₉₇ and polysaccharide specific signals, indicating that the CPS8 glycans were covalently attached to CRM₁₉₇-ComP_{C1} protein.

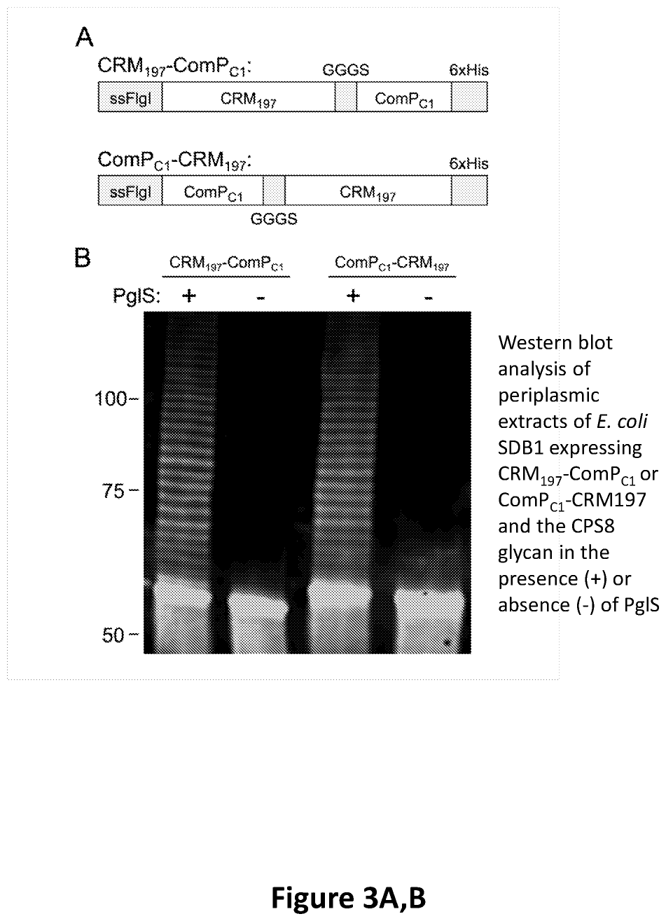

Next, whether the ComP_{C1} glycotag could be moved to another site in the CRM₁₉₇ fusion was tested. As such, a new construct was designed placing ComP_{C1} N-terminal to the CRM₁₉₇ coding region (FIG. 3A). The FlgI secretion signal was placed immediately N-terminal to ComP_{C1} glycosylation fragment and CRM₁₉₇ was C-terminally tagged with hexahistidine. E. coli SDB1 cells expressing the CPS8 glycan along with PglS and the ComP_{C1}-CRM₁₉₇ carrier were cultured in shake flasks and harvested after 24 hours. As seen in FIG. 3B, Western blot analysis of periplasmic extracts probing with an anti-His antibody showed that the ComP_{C1}-CRM₁₉₇ was also glycosylated by PglS. The average number of CPS8 repeat units and glycosylation efficiency of both fusions was comparable, indicating that ComP_{C1} glycotag can be placed at the N- or C-terminus of a carrier protein.

### Identification of an 11 Amino Acid ComP₁₁₀₂₆₃ Sequon Sufficient for PglS Glycosylation.

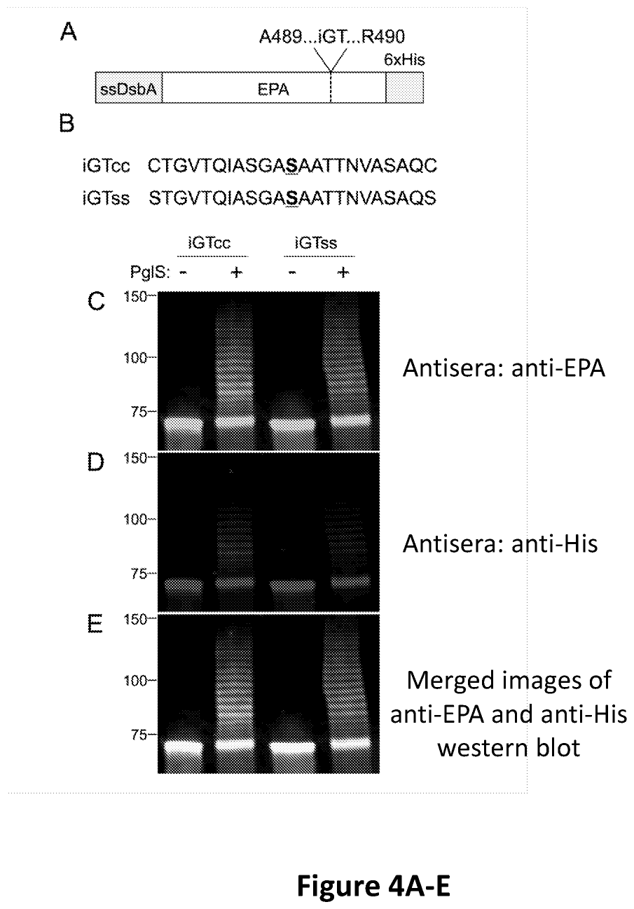

While prior reports indicated that Cys71 and Cys93 were required for glycosylation of fusion proteins containing a ComP₁₁₀₂₆₄ glycosylation fragment translationally fused at the C-terminus of EPA (e.g., FIG. 1C, FIG. 1D, and FIG. 1E), these data do not ascertain whether the two cysteine residues and the putative disulfide bridge formed between them are absolutely required for glycosylation by PglS in all circumstances. The N-linking sequon recognized by PglB has been engineered into multiple sites on surface loops of EPA and used as an “internal” glycotag (Ihssen, J. et al., 2010). In order to determine whether Cys71 and Cys93 of ComP₁₁₀₂₆₄ are necessary for PglS glycosylation, the entire 23 amino acid ComP₁₁₀₂₆₄ glycosylation fragment spanning Cys71 to Cys93-referred to herein as the iGT_{CC} for internal GlycoTag—cysteine-cysteine—was integrated internal of the EPA amino acid sequence. The ComP₁₁₀₂₆₄ iGT_{CC} was inserted between residues Ala489 and Arg490 of EPA, which is in a p-turn structure on the surface of the catalytic domain (FIG. 4A). As a control, a variant of the iGT_{CC} ComP glycosylation fragment containing serine residues instead of cysteine residues at positions 71 and 93 of ComP termed iGTss (“serine-serine”) was also integrated. This iGTSS ComP glycosylation fragment was also integrated between residues Ala489 and Arg490 of EPA. Serine residues are hypothesized to contribute a similar steric bulk as the cysteine residues, but are unable to oxidize and form a disulfide bond (FIG. 4B). The ability of PglS to transfer CPS8 to the EPA_{iGTcc}, or EPA_{iGTss} was assessed in a three-plasmid system as described above. As seen in FIG. 4C and FIG. 4D, both the cysteine-cysteine and serine-serine variants of EPA_{iGT} were glycosylated, demonstrating that Cys71 and Cys93 (and the putative disulfide bond formed between them) are not required for glycosylation by PglS when the ComP fragment is introduced internal of the EPA protein.

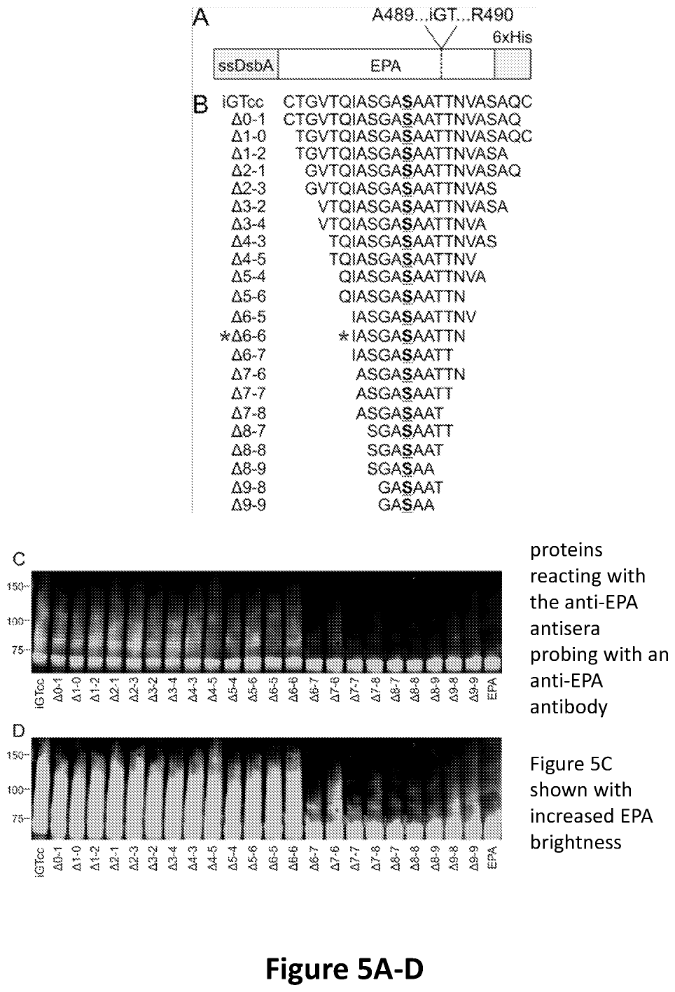

Since the cysteine residues are not necessary for PglS dependent glycosylation only when the ComP glycosylation fragment is integrated internal of the fusion protein, it was contemplated that a shorter ComP glycosylation fragment representing the minimal 0-linking ComP sequon could be found within the 23-amino acid ComP glycosylation fragment spanning Cys71 to Cys93. To investigate this, shorter variants of the iGT_{CC} ComP glycosylation fragment integrated between EPA residues Ala489-Arg490 were generated in order to identify which ComP residues were necessary for glycosylation. Alternate single amino acids were deleted from either side of the 23-amino acid iGTcc, generating 22 truncated variants that each contained Ser82, the site of PglS glycosylation (FIG. 5A and FIG. 5B). These variants were named after the number of deleted residues from either side of the iGTcc, e.g. Δ3-4 corresponds to a deletion of three amino acids from the N-terminal side of iGT_{CC} and a four amino acid deletion from the C-terminal side. The shortest variant generated was five amino acids long. These truncated EPA-iGTcc variants were tested for bioconjugation with CPS8 and PglS in shake flasks under the same conditions as the preceding experiments. As a negative control, we included a construct expressing only the EPA coding sequence along with DsbA secretion and hexahistidine tags.

FIG. 5C shows robust glycosylation for all EPA fusion proteins containing ComP glycosylation fragments that were at least 11 amino acids in length was observed. The glycosylation ratio was comparable to the 23 amino acid iGT_{CC} ComP glycosylation fragment, suggesting modest truncations on either side of Ser82 do not have a significant impact on the glycosylation efficiency by PglS. Although these fusion proteins were glycosylated, a mild decrease in glycosylation efficiency was observed as the iGT ComP glycosylation fragment amino acid sequence was shortened. The shortest internal ComP glycosylation fragment that was efficiently glycosylated was iGTΔ6-6 having the sequence IASGASAATTN (SEQ ID NO: 109); FIG. 5C). Removal of either the N-terminal isoleucine residue (iGTΔ7-6; SEQ ID NO: 121) or C-terminal asparagine residue (iGTΔ6-7; SEQ ID NO: 110) dramatically reduced the glycosylation efficiency of the carrier protein, suggesting that these residues play an important role in PglS glycosylation. Variants smaller than iGTΔ6-6 mostly showed minimal glycosylation, the best of these being iGTΔ7-6 with sequence ASGASAATTN (SEQ ID NO: 121). Interestingly, a small amount of higher molecular weight laddering was also observed in fusion proteins containing the smallest ComP glycosylation fragments, iGTΔ9-8 (SEQ ID NO: 146) and iGTΔ9-9 (SEQ ID NO: 147) (FIG. 5D), suggesting that these six and five amino acid variants, respectively, were glycosylated by PglS at very low levels. This implies that the ComP₁₁₀₂₆₄ glycosylation sequon recognized by PglS can be as small as five amino acids in size.

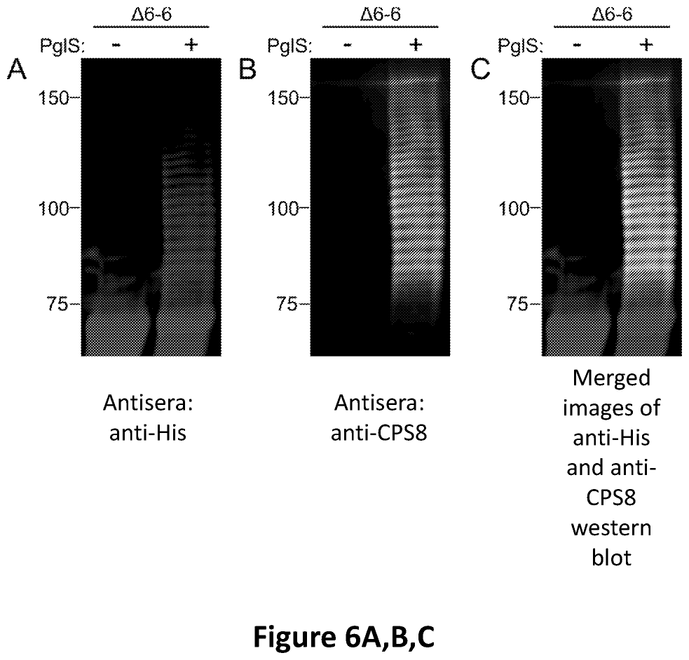

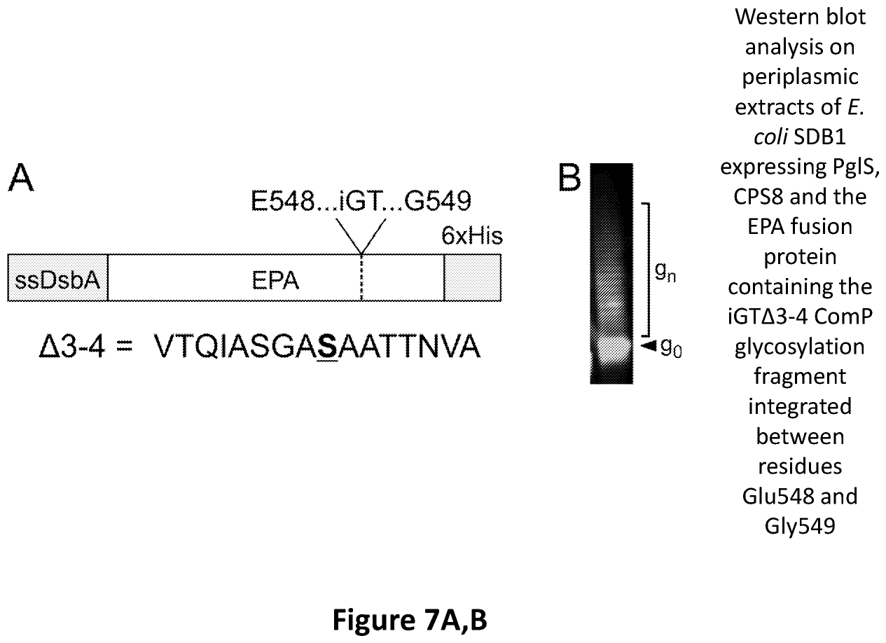

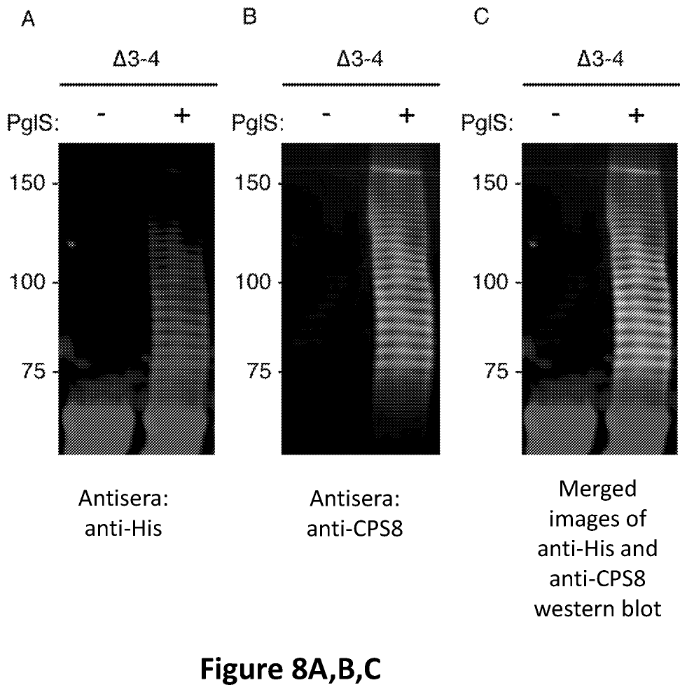

Next, the CPS8 glycosylated EPA fusion protein containing the iGTΔ6-6 ComP glycosylation fragment located between residues Ala489-Arg490 was purified from whole-cell lysates using a Ni-affinity chromatography and performed western blot analysis on the eluate using antisera specific to either the EPA protein or the CPS8 glycan. The results of these experiments clearly show that the EPA fusion protein containing the iGTΔ6-6 ComP glycosylation fragment located between residues Ala489-Arg490 was being glycosylated with CPS8 by PglS (FIG. 6A, FIG. 6B, and FIG. 6C). Overall, these experiments show that ComP₁₁₀₂₆₄ glycosylation fragment can be shortened from the 117 amino acid ComP₁₁₀₂₆₄ to a sequon as short as or shorter than 11 amino acids while maintaining glycosylation. These results unexpectedly show that the cysteine residues corresponding to Cys71 and Cys93 of ComP₁₁₀₂₆₄, previously shown to be required when fused at the C-terminus, are not required for PglS dependent glycosylation when the ComP glycosylation fragment is integrated internal of the fusion protein.

The preceding iGT truncation series was tested at one internal site on EPA between residues Ala489 and Arg490. Next, a second site between EPA residues Glu548 and Gly549 and incorporated the iGTΔ3-4 ComP glycosylation fragment (SEQ ID NO: 71) was tested. Like the first site, the second site is found on a surface-exposed loop in the catalytic domain of EPA. This alternately tagged variant for bioconjugation with CPS8 and PglS was tested under the same conditions as the other truncations. It was observed that this construct was glycosylated with CPS8 at a similar efficiency as when iGTΔ3-4 was placed in the first site on EPA. The CPS8 glycosylated EPA fusion protein containing the iGTΔ3-4 ComP glycosylation fragment located between residues Glu548 and Gly549 was then purified from whole-cell lysates using a Ni-affinity chromatography and performed Western blot analysis on the eluate using antisera specific to either the EPA protein or the CPS8 glycan. The results of these experiments again show that the EPA fusion protein containing the iGTΔ3-4 ComP glycosylation fragment located between residues Glu548 and Gly549 was being glycosylated with CPS8 by PglS. Overall, these experiments show that ComP₁₁₀₂₆₄ glycosylation fragment can be shortened from the 117 amino acid ComP₁₁₀₂₆₄ to a sequon as short as 11 amino acids or shorter while maintaining glycosylation. These results unexpectedly show that the cysteine residues corresponding to Cys71 and Cys93 of ComP₁₁₀₂₆₄ are not required for PglS dependent glycosylation when the ComP glycosylation fragment is integrated internal of the fusion protein.

Provided herein are glycoconjugates comprising an oligo- or polysaccharide linked to a fusion protein. In certain embodiments, the oligo- or polysaccharide is covalently linked to the fusion protein. The fusion protein comprises a glycosylation fragment of a ComP protein (as described in detail elsewhere herein). In certain embodiments of a glycoconjugate of this disclosure, the oligo- or polysaccharide comprises a glucose at its reducing end.

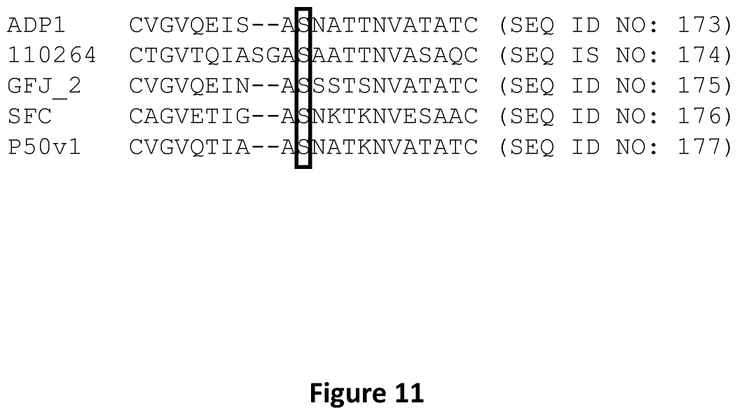

ComP is glycosylated on a serine (S) residue. This serine residue corresponds to position 82 of SEQ ID NO: 1 (ComP₁₁₀₂₆₄: ENV58402.1). This serine residue is conserved in ComP proteins and, for example, corresponds to position 84 of SEQ ID NO: 2 (ComP_{ADP1}: AAC45886.1). Thus, in certain aspects, a fusion protein (and thus the glycoconjugate) is glycosylated with an oligo- or polysaccharide on a ComP glycosylation fragment at a serine residue corresponding to the serine residue at position 84 of SEQ ID NO: 2 (ComP_{ADP1}: AAC45886.1) or corresponding to the serine residue at position 82 of SEQ ID NO: 1 (ComP₁₁₀₂₆₄: ENV58402.1). FIG. 11 shows an alignment of a region of ComP sequences including the serine (S) residue (boxed) corresponding to the serine residue at position 82 of SEQ ID NO: 1 (ComP₁₁₀₂₆₄: ENV58402.1), which is conserved across the ComP sequences.

One of ordinary skill in the art would recognize that by aligning ComP sequences with SEQ ID NO: 1, (e.g., either full sequences or partial sequences) the conserved serine residue of a non-SEQ ID NO: 1 ComP protein, corresponding to the serine residue at position 82 of SEQ ID NO: 1, can be identified. Further, one of ordinary skill in the art would recognize that by aligning ComP sequences with SEQ ID NO: 1, other residues, regions, and/or features corresponding to residues, regions, and/or features of SEQ ID NO: 1 as referred to herein can be identified in the non-SEQ ID NO: 1 ComP sequence and referenced in relation to SEQ ID NO: 1. And, while reference is generally made herein to SEQ ID NO: 1, by analogy, reference can similarly be made to any residue, region, feature and the like of any ComP sequence disclosed herein, for example, in reference to SEQ ID NO: 2.

A ComP protein is a protein that has been identified as a ComP protein consistent with the description provided herein. For example, representative examples of ComP proteins include, but are not limited to: AAC45886.1 ComP [Acinetobacter sp. ADP1]; ENV58402.1 hypothetical protein F951_00736 [Acinetobacter soli CIP 110264]; APV36638.1 competence protein [Acinetobacter soli GFJ-2]; PKD82822.1 competence protein [Acinetobacter radioresistens 50v1]; SNX44537.1 type IV pilus assembly protein PilA [Acinetobacter puyangensis ANC 4466]; OAL75955.1 competence protein [Acinetobacter sp. SFC]; ComPP5312; and ComP_{ANT_H59}. In certain aspects, a ComP protein comprises an amino acid sequence that is at least 50%, 60%, 70%, 75%, 80%, 85%, 90%, 95%, 96%, 97%, 98%, 99%, or 100% identical to SEQ ID NO: 2 (ComP_{ADP1}) or SEQ ID NO: 1 (ComP₁₁₀₂₆₄) and contains a serine residue corresponding to the conserved serine residue at position 84 of SEQ ID NO: 2 or at position 82 of SEQ ID NO: 1. SEQ ID NO: 2 comprises a leader sequence of 28 amino acids. In certain aspects, a ComP protein comprises an amino acid sequence that is at least 50%, 60%, 70%, 75%, 80%, 85%, 90%, 95%, 97%, 98%, 99%, or 100% identical to SEQ ID NO: 10 (ComPΔ28_{ADP1}), SEQ ID NO: 9 (ComPΔ28₁₁₀₂₆₄), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}), SEQ ID NO: 15 (ComPΔ28_{P5312}), or SEQ ID NO: 16 (ComPΔ29_{ANT_H59}) that do not include the amino acid leader sequence but do contain a serine residue corresponding to the conserved serine residue at position 82 of SEQ ID NO: 1 (ComP₁₁₀₂₆₄: AAC45886.1). In certain aspects, a ComP protein comprises an amino acid sequence that is at least 50%, 60%, 70%, 75%, 80%, 85%, 90%, 95%, 97%, 98%, 99%, or 100% identical to SEQ ID NO: 9 (ComPΔ28110264) that does not include the 28 amino acid leader sequence but does contain a serine residue corresponding to the conserved serine residue at position 82 of SEQ ID NO: 1 (ComP₁₁₀₂₆₄). In certain aspects, the ComP protein comprises SEQ ID NO: 10 (ComPΔ28_{ADP1}), SEQ ID NO: 9 (ComPΔ28110264), SEQ ID NO: 11 (ComPΔ28_{GFJ-2}), SEQ ID NO: 12 (ComPΔ28P₅₀ᵥ₁), SEQ ID NO: 13 (ComPΔ284466), SEQ ID NO: 14 (ComPΔ28_{SFC}), SEQ ID NO: 15 (ComPΔ28_{P5312}), or SEQ ID NO: 16 (ComPΔ29_{ANT_H59}). In certain aspects, the ComP protein is SEQ ID NO: 2 (ComP_{ADP1}: AAC45886.1), SEQ ID NO: 1 (ComP₁₁₀₂₆₄: ENV58402.1), SEQ ID NO: 3 (ComP_{GFJ-2}: APV36638.1), SEQ ID NO: 4 (ComP₅₀ᵥ₁: PKD82822.1), SEQ ID NO: 5 (ComP₄₄₆₆: SNX44537.1), SEQ ID NO: 6 (ComP_{SFC}: OAL75955.1), SEQ ID NO: 7 (ComP_{P5312}), or SEQ ID NO: 8 (ComP_{ANT_H59}).

Provided for herein is a glycoconjugate comprising an oligo- or polysaccharide covalently linked to a fusion protein wherein the fusion protein comprises a ComP protein (ComP) glycosylation fragment. In certain embodiments, the ComP glycosylation fragment does not contain a cysteine (C) residue corresponding to the conserved cysteine (C) residue at position 71 of ComP₁₁₀₂₆₄ (SEQ ID NO: 1). In certain embodiments, the ComP glycosylation fragment does not contain a cysteine (C) residue corresponding to the conserved cysteine (C) residue at position 93 of ComP₁₁₀₂₆₄ (SEQ ID NO: 1). As described in greater detail herein, the fusion protein is glycosylated with the oligo- or polysaccharide on the ComP glycosylation fragment at serine residue corresponding to the conserved serine residue at position 82 of ComP₁₁₀₂₆₄ (SEQ ID NO: 1). In certain embodiments, the ComP glycosylation fragment is located internally within the fusion protein. Further, in certain embodiments, the ComP glycosylation fragment portion of the fusion protein is solvent (or surface)-exposed and/or is integrated into a C₁₀ β-turn, β-turn, β-twist, β-loop, U turn, reverse turn, chain reversal, or a hairpin loop of the fusion protein.

Because it has been discovered that when the ComP glycosylation fragment is located internally within the fusion protein, it does not require the flanking cysteine residues for glycosylation, the ComP glycosylation fragments disclosed herein can be shorter than previously believed. In certain embodiments, the ComP glycosylation fragment can be shorter than 23, 22, 21, 20, 19, 18, 17, 16, 15, 14, 13, 12, 11, 10, 9, 8, 7, or 6 amino acids long, as long as it comprises a serine residue corresponding to the conserved serine residue at position 82 of ComP₁₁₀₂₆₄ (SEQ ID NO: 1). In certain embodiment, the ComP glycosylation fragment has a length of from any one of 5, 6, 7, 8, 9, 10, 11, 12, 13, 14, or 15 to any one of 6, 7, 8, 9, 10, 11, 12, 13, 14, 15, 16, 17, 18, 19, 20, 21, or 22 amino acids in length. In certain embodiments, the fragment has at least 1, 2, 3, 4, 5, 6, 7, 8, 9, 10, or 11 amino acid residues of the ComP protein N-terminal to the serine residue corresponding to the conserved serine residue at position 82 of SEQ ID NO: 1, e.g., XₙS[Y], wherein n is at least 1, 2, 3, 4, 5, 6, 7, 8, 9, 10, or 11 amino acid residues of the ComP protein. In certain embodiments, the fragment has at least 1, 2, 3, 4, 5, 6, 7, 8, 9, 10, or 11 amino acid residues of the ComP protein C-terminal to the serine residue corresponding to the conserved serine residue at position 82 of SEQ ID NO: 1, e.g., [X]SYₙ, wherein n is at least 1, 2, 3, 4, 5, 6, 7, 8, 9, 10, or 11 amino acid residues of the ComP protein. Further, in certain embodiments, the amino acid sequence of the ComP glycosylation fragment does not extend in the N-terminus direction beyond the amino acid residue corresponding to position 72 of ComP₁₁₀₂₆₄ (SEQ ID NO: 1) and/or does not extend in the C-terminus beyond the amino acid residue corresponding to position 92 of ComP₁₁₀₂₆₄ (SEQ ID NO: 1).

Consistent with a ComP protein of this disclosure, in certain embodiments, a ComP protein from which the ComP glycosylation fragment is derived comprises an amino acid sequence that is at least 50%, 60%, 70%, 75%, 80%, 85%, 90%, 95%, 96%, 97%, 98%, 99%, or 100% identical to SEQ ID NO: 9 (ComPΔ28₁₁₀₂₆₄) SEQ ID NO: 10 (ComPΔ28_{ADP1}), SEQ ID NO: 11 (ComPΔ28G_{FJ}-2), SEQ ID NO: 12 (ComPΔ28_{P50v1}), SEQ ID NO: 13 (ComPΔ284466), SEQ ID NO: 14 (ComPΔ28_{SFC}); SEQ ID NO: 15 (ComPΔ28_{P5312}), or SEQ ID NO: 16 (ComPΔ29_{ANT_H59}). In certain embodiments, the ComP protein from which the ComP glycosylation fragment is derived comprises SEQ ID NO: 9 (ComPΔ28₁₁₀₂₆₄), SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59}).

In certain embodiments of the glycoconjugate of this disclosure, the ComP glycosylation fragment comprises or consists of the amino acid consensus sequence of:

(SEQ ID NO: 17)
X₁GVX₄X₅X₆X₇X₈X₉ASX₁₂X₁₃TX₁₅NVX₁₈X₁₉X₂₀X₂₁;
(SEQ ID NO: 196)
CX₁GVX₄X₅X₆X₇X₈X₉ASX₁₂X₁₃TX₁₅NVX₁₈X₁₉X₂₀X₂₁;
or
(SEQ ID NO: 197)
X₁GVX₄X₅X₆X₇X₈X₉ASX₁₂X₁₃TX₁₅NVX₁₈X₁₉X₂₀X₂₁C;

wherein: X₁ is V, T, A, or I;
   X₄ is Q, T, E, A, or S;
   X₅ is E, Q, T, or L;
   X₆ is I or V;
   X₇ is S, N, A, or G;
   X₈ is S or no amino acid;
   X₉ is G, D, or no amino acid;
   X₁₂ is N, S, or A;
   X₁₃ is A, S, or K;
   X₁₅ is T, S, or K;
   X₁₈ is A, E, Q, or L;
   X₁₉ is T, S, or K;
   X₂₀ is A or S; and
   X₂₁ is T, Q, A, or V;
      or a fragment of thereof of at least 5, 6, 7, 8, 9, 10, 11, 12, 13, 14, or 15 amino acids in length comprising the serine (S) residue corresponding to position 11 of SEQ ID NO: 17. In certain embodiments, the fragment has at least 1, 2, 3, 4, 5, 6, 7, or 8 amino acid residues N-terminal to the serine (S) residue corresponding to position 11 of SEQ ID NO: 17. In certain embodiments, the fragment has at least 1, 2, 3, 4, 5, 6, 7, or 8 amino acid residues C-terminal to the serine (S) residue corresponding to position 11 of SEQ ID NO: 17. But, the ComP glycosylation fragment does not contain a cysteine (C) residue corresponding to the conserved cysteine (C) residue at position 71 of ComP₁₁₀₂₆₄ (SEQ ID NO: 1) and/or the ComP glycosylation fragment does not contain a cysteine (C) residue corresponding to the conserved cysteine (C) residue at position 93 of ComP₁₁₀₂₆₄ (SEQ ID NO: 1).

Certain embodiments provide for a ComP glycosylation fragment that is a variant of the amino acid consensus sequence of SEQ ID NO: 17, SEQ ID NO: 196, or SEQ ID NO: 197, or the fragment thereof, having 1, 2, 3, 4, 5, 6 or 7 amino acid substitutions, additions, and/or deletions, wherein the variant maintains the serine (S) residue corresponding to position 11 of SEQ ID NO: 17 and wherein the variant does not contain a cysteine (C) residue corresponding to the conserved cysteine (C) residue at position 71 of ComP₁₁₀₂₆₄ (SEQ ID NO: 1) and/or the variant does not contain a cysteine (C) residue corresponding to the conserved cysteine (C) residue at position 93 of ComP₁₁₀₂₆₄ (SEQ ID NO: 1). One of ordinary skill in the art will understand that the number of amino acid substitutions, additions, and/or deletions that can be tolerated within a sequence without abolishing its function (e.g., ability to function as a sequon) can depend on the length of the sequence. For example, a six amino acid long sequence will tolerate less changes than a 21 amino acid long sequence.

Whether a ComP glycosylation fragment can be glycosylated (including subfragments of a fragment and variants as disclosed herein and collectively referred to as ComP glycosylation fragments), and the efficiency of glycosylation, can be determined such as by methods described herein. In certain embodiments, the ComP glycosylation fragment can be glycosylated when located internally in a fusion protein and/or internally in a carrier protein sequence as described elsewhere herein. Further, in certain embodiments, the ComP glycosylation fragment or variant is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

In certain embodiments, the fusion protein comprises a carrier protein selected from the group consisting of Pseudomonas aeruginosa Exotoxin A (EPA), CRM₁₉₇, cholera toxin B subunit, tetanus toxin C fragment, Haemophilus influenzae Protein D, and a fragment or fragments thereof. For example, in certain embodiments, the Pseudomonas aeruginosa Exotoxin A (EPA) carrier protein comprises the amino acid sequence of SEQ ID NO: 18, or a fragment or fragments thereof. For example, in certain embodiments, the CRM₁₉₇ carrier protein comprises the amino acid sequence of SEQ ID NO: 24, or a fragment or fragments thereof.

As can be understood from this disclosure as a whole, by internally within the fusion protein, it is meant that the ComP fusion protein is not located at the C-terminal end or the N-terminal end of the fusion protein, not including any C-terminal leader sequence or N-terminal tag (e.g., His-Tag), or the like. For example:

### N-Terminal, not Internal

Leader sequence—ComP glycosylation fragment—Carrier protein

### C-Terminal, not Internal

Carrier protein—ComP glycosylation fragment—His-Tag

### Internal

Leader Sequence—Carrier protein—ComP glycosylation fragment—Carrier protein-His-Tag
   In certain embodiments, the ComP glycosylation fragment can be attached to the carrier protein sequence via an amino acid linker.

Further, in certain embodiments, the ComP glycosylation fragment can be inserted into the sequence of a carrier protein rather than between carrier proteins. For example, in certain embodiments:
(i) the ComP glycosylation fragment is inserted between Ala489 and Arg490 relative to the PDB entity 1IKQ of Pseudomonas aeruginosa Exotoxin A (EPA) (SEQ ID NO: 19);
(ii) the ComP glycosylation fragment is inserted between Glu548 and Gly549 relative to the PDB entity 1IKQ of Pseudomonas aeruginosa Exotoxin A (EPA) (SEQ ID NO:20);
(iii) the ComP glycosylation fragment is inserted between Ala122 and Gly123 relative to the PDB entity 1IKQ of Pseudomonas aeruginosa Exotoxin A (EPA) (SEQ ID NO: 21);
(iv) the ComP glycosylation fragment is inserted between Thr355 and Gly356 relative to the PDB entity 1IKQ of Pseudomonas aeruginosa Exotoxin A (EPA) (SEQ ID NO: 22); or
(v) the ComP glycosylation fragment is inserted between Lys20 and Asp21 relative to the PDB entity 1IKQ of Pseudomonas aeruginosa Exotoxin A (EPA) (SEQ ID NO: 23).

Further, in certain embodiments, the ComP glycosylation fragment can be inserted into the sequence of a carrier protein rather than between carrier proteins. For example, in certain embodiments:
(i) the ComP glycosylation fragment is inserted between Asn481 and Gly482 relative to the PDB entity 4AE0 of CRM₁₉₇ (SEQ ID NO: 25);
(ii) the ComP glycosylation fragment is inserted between Asp392 and Gly393 relative to the PDB entity 4AE0 of CRM₁₉₇ (SEQ ID NO: 26);
(iii) the ComP glycosylation fragment is inserted between Glu142 and Gly143 relative to the PDB entity 4AE0 of CRM₁₉₇ (SEQ ID NO: 27);
(iv) the ComP glycosylation fragment is inserted between Asp129 and Gly130 relative to the PDB entity 4AE0 of CRM₁₉₇ (SEQ ID NO: 28); or
(v) the ComP glycosylation fragment is inserted between Asn69 and Glu70 relative to the PDB entity 4AE0 of CRM₁₉₇ (SEQ ID NO: 29).

In certain embodiments, ComP glycosylation fragments can be located between carrier proteins and also inserted into the sequence of a carrier protein(s) within one fusion protein. In certain embodiments, a ComP glycosylation fragment can be located internally and one or more ComP glycosylation fragments can be located at the C-terminal and/or N-terminal end that are sufficient for glycosylation at such location.

An aspect of this disclosure is that a fusion protein can be designed to comprise multiple ComP glycosylation fragments such as to increase the immunogenicity of the glycosylated fusion protein/glycoconjugate. In certain embodiments, the fusion protein comprises two or more, three or more, four or more, five or more, six or more, eight or more, ten or more, fifteen or more, or twenty or more ComP glycosylation fragments. In certain embodiments, the fusion protein does not comprise more than three, more than five, more than ten, more than fifteen, more than twenty, or more than twenty five ComP glycosylation fragments. The identity of the ComP glycosylation fragments can also be controlled. For example, in certain embodiments, a plurality of ComP glycosylation fragments of a fusion protein are identical. In certain embodiments, ComP glycosylation fragments of a fusion protein differ from each other. For example, in certain embodiments, at least three, at least four, or at least five of the ComP glycosylation fragments of a fusion protein all differ from each other. For example, in certain embodiments, none of the ComP glycosylation fragments of a fusion protein are the same.

In certain embodiments, the oligo- or polysaccharide is derived from a saccharide produced by bacteria from the genus Streptococcus. For example, in certain embodiments, the saccharide is a S. pneumoniae, S. agalactiae, or S. suis capsular polysaccharide; in certain embodiments, the saccharide is the serotype 8 capsular polysaccharide from S. pneumoniae; and in certain embodiments, the saccharide is the type Ia, Ib, II, III, IV, V, VI, VII, VIII, or X capsular polysaccharide from S. agalactiae.

In certain embodiments, the oligo- or polysaccharide is derived from a saccharide produced by the bacteria from the genus Klebsiella. For example, in certain embodiments, the saccharide is a K. pneumoniae, K. varricola, K. michinganenis, or K. oxytoca capsular polysaccharide; and in certain embodiments, the saccharide is a K. pneumoniae, K. varricola, K. michinganenis, or K. oxytoca O-antigen polysaccharide.

In certain embodiments, the glycoconjugate is produced in vivo, for example: in a bacterial cell; in Escherichia coli; in a bacterium from the genus Klebsiella; and/or wherein the bacterial species is K. pneumoniae, K. varricola, K. michinganenis, or K. oxytoca.

Provided for herein is a glycoconjugate as described above (e.g., the ComP glycosylation fragment does not contain a cysteine (C) residue corresponding to the conserved cysteine (C) residue at position 71 of ComP₁₁₀₂₆₄ (SEQ ID NO: 1) and/or the ComP glycosylation fragment does not contain a cysteine (C) residue corresponding to the conserved cysteine (C) residue at position 93 of ComP₁₁₀₂₆₄ (SEQ ID NO: 1)), wherein the ComP glycosylation fragment comprises or consists of an amino acid sequence of SEQ ID NO: 32-163, or 164. Provided for herein is a glycoconjugate as described above (e.g., the ComP glycosylation fragment does not contain a cysteine (C) residue corresponding to the conserved cysteine (C) residue at position 71 of ComP₁₁₀₂₆₄ (SEQ ID NO: 1) and/or the ComP glycosylation fragment does not contain a cysteine (C) residue corresponding to the conserved cysteine (C) residue at position 93 of ComP₁₁₀₂₆₄ (SEQ ID NO: 1)), wherein the ComP glycosylation fragment comprises or consists of an amino acid sequence of:

iGTccΔ0-1
(SEQ ID NO: 32)
CTGVTQIASGASAATINVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATTNVASA;
iGTccΔ2-1
(SEQ ID NO: 56)
GVTQIASGASAATTNVASAQ;
iGTccΔ2-2
(SEQ ID NO: 57)
GVTQIASGASAATTNVASA;
iGTccΔ2-3
(SEQ ID NO: 58)
GVTQIASGASAATTNVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN

Also provided for herein is a ComP glycosylation fragment that is a variant of any of the above disclosed ComP glycosylation fragments having 1, 2, 3, 4, 5, 6, or 7 amino acid substitutions, additions, and/or deletions, wherein the variant maintains the serine residue corresponding to the conserved serine residue at position 82 of SEQ ID NO: 1 and wherein the variant does not contain a cysteine (C) residue corresponding to the conserved cysteine (C) residue at position 71 of ComP₁₁₀₂₆₄ (SEQ ID NO: 1) and/or the variant does not contain a cysteine (C) residue corresponding to the conserved cysteine (C) residue at position 93 of ComP₁₁₀₂₆₄ (SEQ ID NO: 1).

Whether a ComP glycosylation fragment can be glycosylated (including subfragments of a fragment and variants as disclosed herein and collectively referred to as ComP glycosylation fragments), and the efficiency of glycosylation, can be determined such as by methods described herein. In certain embodiments, the ComP glycosylation fragment can be glycosylated when located internally in a fusion protein and/or internally in a carrier protein sequence as described elsewhere herein. Further, in certain embodiments, the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

In certain embodiments, the glycoconjugate is a conjugate vaccine. Thus, this disclosure in certain embodiments is directed to and provides for a conjugate vaccine. In certain embodiments the conjugate vaccine is a vaccine against Streptococcus pneumoniae serotype 8. In certain embodiments, the conjugate vaccine induces an immune response when administered to a subject. In certain embodiments, the immune response elicits long term memory (memory B and T cells), is an antibody response, and is optionally a serotype-specific antibody response. In certain embodiments, the antibody response is an IgG or IgM response. In certain embodiments, the antibody response is an IgG response; optionally an IgG1 response. And, in certain embodiments, the conjugate vaccine generates immunological memory in a subject administered the vaccine.

Whereas the above describes a glycoconjugate comprising a ComP glycosylation fragment that comprises an isolated fragment of a ComP protein, it is understood that this disclosure also explicitly provides for a ComP glycosylation fragment consistent with any and all description of a ComP glycosylation fragment provided anywhere herein, including in the appended Claims below, e.g., wherein the ComP glycosylation fragment does not contain a cysteine residue corresponding to the conserved cysteine residue at position 71 of ComP₁₁₀₂₆₄ (SEQ ID NO: 1) and/or does not contain a cysteine residue corresponding to the conserved cysteine residue at position 93 of ComP₁₁₀₂₆₄ (SEQ ID NO: 1) and wherein the ComP glycosylation fragment comprises the serine residue corresponding to the conserved serine residue at position 82 of ComP₁₁₀₂₆₄ (SEQ ID NO: 1).

Provided for herein is as fusion protein comprising a ComP glycosylation fragment of this disclosure. In certain embodiments, the fusion protein is glycosylated by an oligo- or polysaccharide at a serine residue on the glycosylation fragment corresponding to the serine ComP glycosylation fragment residue at position 82 of SEQ ID NO: 1 (ComP₁₁₀₂₆₄). Further, whereas the above describes a glycoconjugate comprising a ComP glycosylation fragment that comprises a fusion protein, it is understood that this disclosure also explicitly provides for a fusion protein consistent with any and all description of a fusion protein provided anywhere herein, including in the appended Claims below. In certain embodiments, the fusion protein comprises a carrier protein selected from the group consisting of Pseudomonas aeruginosa Exotoxin A (EPA), CRM₁₉₇, cholera toxin B subunit, tetanus toxin C fragment, Haemophilus influenzae Protein D, and a fragment or fragments thereof.

Also provided for herein is a method of in vivo conjugation of an oligo- or polysaccharide to an acceptor polypeptide. In certain embodiments, the method comprises culturing a host cell comprising the components necessary for the conjugation of the oligo- or polysaccharide to the polypeptide. In general, these components are the oligosaccharyltransferase, the acceptor polypeptide to be glycosylated, and the oligo- or polysaccharide. The method comprises covalently linking an oligo- or polysaccharide to the acceptor polypeptide (fusion protein of this disclosure) with a PglS oligosaccharyltransferase (OTase), wherein the acceptor polypeptide comprises a ComP glycosylation fragment as described herein. In certain embodiments, the PglS OTase is PglS₁₁₀₂₆₄ (SEQ ID NO: 165), PglS_{ADP1} (SEQ ID NO: 166), PglS_{GFJ-2} (SEQ ID NO: 167), PglS₅₀ᵥ₁ (SEQ ID NO: 168), PglS₄₄₆₆ (SEQ ID NO: 169), PglS_{SFC} (SEQ ID NO: 170), Pgl_{SP5312} (SEQ ID NO: 171), or PglS_{ANT_H59} (SEQ ID NO: 172). In certain embodiments, the oligo- or polysaccharide is linked to the ComP glycosylation fragment at a serine (S) residue corresponding to the serine residue at position 82 of SEQ ID NO: 1 (ComP₁₁₀₂₆₄). In certain embodiments, the in vivo conjugation occurs in a host cell. In certain aspects, the glycoconjugate is produced in a bacterial cell, a fungal cell, a yeast cell, an avian cell, an algal cell, an insect cell, or a mammalian cell. In certain embodiments, the host cell is a bacterial cell, e.g.: in Escherichia coli; in a bacterium from the genus Klebsiella; the bacterial species is K. pneumoniae, K. varricola, K. michinganenis, or K. oxytoca. Certain embodiments comprise culturing a host cell that comprises: (a) a genetic cluster encoding for the proteins required to synthesize the oligo- or polysaccharide; (b) a PglS OTase; and (3) the acceptor polypeptide. In certain embodiments, the production of the oligo- or polysaccharide is enhanced by the K. pneumoniae transcriptional activator rmpA (K. pneumoniae NTUH K-2044) or a homolog of the K. pneumoniae transcriptional activator rmpA (K. pneumoniae NTUH K-2044). In certain embodiments, the method further comprises expressing and/or providing such a transcriptional activator in the host cell along with the other components.

In certain aspects, the glycoconjugate is produced in a cell free system. Examples of the use of a cell free system utilizing OTases other than PglS can be found in WO2013/067523A1, which in incorporated herein by reference.

Also provided for is a host cell comprising (a) a genetic cluster encoding for the proteins required to synthesize an oligo- or polysaccharide; (b) a PglS OTase; and (3) an acceptor polypeptide comprising a ComP glycosylation fragment of this disclosure. In certain embodiments, the acceptor polypeptide is a fusion protein. In certain embodiments, the host cell comprises a nucleic acid encoding the PglS OTase. In certain embodiments, the host cell comprises a nucleic acid encoding the acceptor polypeptide.

Also provided for herein is an isolated nucleic acid encoding a ComP glycosylation fragment and/or a fusion protein of this disclosure. In certain embodiments, the nucleic acid is a vector. In certain embodiments, a host cell comprises the isolated nucleic acid.

A glycoconjugate of this invention may have one of numerous uses including, but not limited to, use as a conjugate vaccine. Thus in certain methods, a conjugate vaccine is produced. In certain embodiments, a composition comprising the conjugate vaccine or the fusion protein of this disclosure and an adjuvant. For example, in certain embodiments, the conjugate vaccine is a vaccine against Streptococcus pneumoniae serotype 8, Streptococcus pneumoniae serotype 1, Streptococcus pneumoniae serotype 2, Streptococcus pneumoniae serotype 4, Streptococcus pneumoniae serotype 5, Streptococcus pneumoniae serotype 6A, Streptococcus pneumoniae serotype 6B, Streptococcus pneumoniae serotype 7F, Streptococcus pneumoniae serotype 9N, Streptococcus pneumoniae serotype 9V, Streptococcus pneumoniae serotype 10A, Streptococcus pneumoniae serotype 11A, Streptococcus pneumoniae serotype 12F, Streptococcus pneumoniae serotype 14, Streptococcus pneumoniae serotype 15B, Streptococcus pneumoniae serotype 17F, Streptococcus pneumoniae serotype 18C, Streptococcus pneumoniae serotype 19F, Streptococcus pneumoniae serotype 19A, Streptococcus pneumoniae serotype 20, Streptococcus pneumoniae serotype 22F, Streptococcus pneumoniae serotype 23F, Streptococcus pneumoniae serotype 33F, Klebsiella pneumoniae serotype K1, Klebsiella pneumoniae serotype K2, Klebsiella pneumoniae serotype K5, Klebsiella pneumoniae serotype K16, Klebsiella pneumoniae serotype K20, Klebsiella pneumoniae serotype K54, Klebsiella pneumoniae serotype K57, Streptococcus agalactiae serotype Ia, Streptococcus agalactiae serotype Ib, Streptococcus agalactiae serotype II, Streptococcus agalactiae serotype III, Streptococcus agalactiae serotype IV, Streptococcus agalactiae serotype V, Streptococcus agalactiae serotype VI, Streptococcus agalactiae serotype VII, Streptococcus agalactiae serotype VIII, Streptococcus agalactiae serotype IX, Streptococcus pyogenes Group A Carbohydrate, Enterococcus faecalis serotype A, Enterococcus faecalis serotype B, Enterococcus faecalis serotype C, Enterococcus faecalis serotype D, Enterococcus faecium capsular polysaccharide and lipotechoic acid, Moraxella catarrhalis lipooligosaccharide A, Moraxella catarrhalis lipooligosaccharide B, Moraxella catarrhalis lipooligosaccharide C, and Staphylococcus aureus lipotechoic acid. In certain embodiments, the conjugate vaccine is useful because it induces an immune response when administered to a subject. In certain embodiments, the immune response elicits long term memory (memory B and T cells), is an antibody response, and is optionally a serotype-specific antibody response. In certain embodiments, the antibody response is an IgG or IgM response. For example, in certain embodiments the antibody response can be an IgG response, and in certain embodiments, an IgG1 response. In certain embodiments, the conjugate vaccine generates immunological memory in a subject administered the vaccine.

Disclosed herein is a pneumococcal glyconjugate vaccine containing a conventional vaccine carrier that can be produced by isolating a glycoconjugate or a glycosylated fusion protein of this disclosure comprising a ComP glycosylation fragment of this disclosure and combining the isolated glycoconjugate or isolated glycosylated fusion protein with an adjuvant. In certain embodiments, the ComP glycosylation fragment can be added to a conventional carrier protein Pseudomonas aeruginosa Exotoxin A (EPA). It has been demonstrated that in certain embodiments, the glycosylation fragment/carrier fusion protein can be paired with the CPS8 polysaccharide and use of PglS, generating a carrier protein-CPS8 bioconjugate, a first of its kind pneumococcal bioconjugate vaccine. For example, in certain embodiments, an EPA fusion can be paired with the CPS8 polysaccharide and use of PglS, generating an EPA-CPS8 bioconjugate. It has been demonstrated that the EPA-CPS8 bioconjugate vaccine elicited high IgG titers specific to serotype 8 specific that were protective as determined via bactericidal killing. Importantly, vaccination with as little as 100 ng of polysaccharide in the EPA-CPS8 bioconjugate was able to provide protection. Thus, certain embodiments provide for a CPS8 pneumococcal bioconjugate vaccine.

It is contemplated that a conjugate vaccine (such as the EPA vaccine construct) can comprise additional/multiple sites of glycosylation to increase the glycan to protein ratio as well as expand upon the number of serotypes in order to develop a comprehensive pneumococcal bioconjugate vaccine.

In certain embodiments, a glycoconjugate or glycosylated fusion protein disclosed herein is a conjugate vaccine that can be administered to a subject for the prevention and/or treatment of an infection and/or disease. In certain embodiments, the conjugate vaccine is a prophylaxis that can be used, e.g., to immunize a subject against an infection and/or disease. In certain embodiments, the glycoconjugate is associated with (such as in a therapeutic composition) and/or administered with an adjuvant. Certain embodiments provide for a composition (such as a therapeutic composition) comprising a conjugate vaccine described herein and an adjuvant. In certain embodiments, when the conjugate vaccine is administered to a subject, it induces an immune response. In certain embodiments, the immune response elicits long term memory (memory B and T cells). In certain embodiments, the immune is an antibody response. In certain embodiments, the antibody response is a serotype-specific antibody response. In certain embodiments, the antibody response is an IgG or IgM response. In certain embodiments where the antibody response is an IgG response, the IgG response is an IgG1 response. Further, in certain embodiments, the conjugate vaccine generates immunological memory in a subject administered the vaccine.

Certain embodiments also provide for producing a vaccine against an infection and/or disease. In certain embodiments a method comprises isolating a glycoconjugate or fusion protein disclosed herein (conjugate vaccine) and combining the conjugate vaccine with an adjuvant. In certain embodiments, the infection is a localized or systemic infection of skin, soft tissue, blood, or an organ, or is auto-immune in nature. In certain embodiments, the vaccine is a conjugate vaccine against pneumococcal infection. In certain embodiments, the disease is pneumonia. In certain embodiments, the infection is a systemic infection and/or an infection of the blood. In certain embodiments, the subject is a mammal. For example, in certain embodiments, a pig or a human.

Importantly, the aspects disclosed herein are not limited to pneumococcal polysaccharides, but in fact, have vast applicability for generating bioconjugate vaccines for many important human and animal pathogens that are incompatible with PglB and PglL. Notable examples include the human pathogens Klebsiella pneumoniae and Group B Streptococcus as well as the swine pathogen S. suis, all immensely relevant pathogens with no licensed vaccines available.

Provided herein are methods of inducing a host immune response against a pathogen. In certain embodiments, the pathogen is a bacterial pathogen. In certain embodiments, the host is immunized against the pathogen. In certain embodiments, the method comprises administering to a subject in need of the immune response an effective amount of a ComP conjugate vaccine, glycosylated fusion protein, or any other therapeutic/immunogenic composition disclosed herein. Certain embodiments provide a conjugate vaccine, glycosylated fusion protein, or other therapeutic/immunogenic composition disclosed herein for use in inducing a host immune response against a bacterial pathogen and immunization against the bacterial pathogen. Examples of immune responses include but are not limited to an innate response, an adaptive response, a humoral response, an antibody response, cell mediated response, a B cell response, a T cell response, cytokine upregulation or downregulation, immune system cross-talk, and a combination of two or more of said immune responses. In certain embodiments, the immune response is an antibody response. In certain embodiments, the immune response is an innate response, a humoral response, an antibody response, a T cell response, or a combination of two or more of said immune responses.

Also provided herein are methods of preventing or treating a bacterial disease and/or infection in a subject comprising administering to a subject in need thereof a conjugate vaccine, a fusion protein, or a composition disclosed herein. In certain embodiments, the infection is a localized or systemic infection of skin, soft tissue, blood, or an organ, or is auto-immune in nature. In certain embodiments, the disease is pneumonia. In certain embodiments, the infection is a systemic infection and/or an infection of the blood. In certain embodiments disclosed herein, the subject is a vertebrate. In certain embodiments the subject is a mammal such as a dog, cat, cow, horse, pig, mouse, rat, rabbit, sheep, goat, guinea pig, monkey, ape, etc. And, for example, in certain embodiments the mammal is a human.

In any of the embodiments of administration disclose herein, the composition is administered via intramuscular injection, intradermal injection, intraperitoneal injection, subcutaneous injection, intravenous injection, oral administration, mucosal administration, intranasal administration, or pulmonary administration.

In certain embodiments, the glycoconjugate, glycosylated fusion protein, or conjugate vaccine of any of the above claims for use in inducing a host immune response against a bacterial pathogen and/or preventing or treating a bacterial disease and/or infection in a subject.

Immunization with a Glycosylated ComP Bioconjugate Elicits an Immune Response.

T-cell dependent immune responses to conjugate vaccines are characterized by the secretion of high affinity IgG1 antibody (Avci, F. Y., Li, X., Tsuji, M. & Kasper, D. L. Nat Med 17, 1602-1609 (2011)). The immunogenicity of a CPS14-ComP bioconjugate in a murine vaccination model was evaluated (WO/2020/131236, which in incorporated by reference herein in its entirety). Sera collected from mice vaccinated with a CPS14-ComP bioconjugate had a significant increase in CPS14 specific IgG titers but not IgM titers. Further, secondary HRP-tagged anti-IgG subtype antibodies were employed to determine which of the IgG subtypes had elevated titers. IgG1 titers appeared to be higher than the other subtypes.

Next, a second vaccination trial was performed comparing the immunogenicity of a trivalent CPS8-, CPS9V-, and CPS14-ComP bioconjugate to the current standard of care, PREVNAR 13@. Serotypes 9V and 14 are included in PREVNAR 13@ and elevated IgG titers could be seen in PREVNAR 13® immunized mice against these two serotypes. The monovalent immunization against serotype 14 also showed significant induction of serotype specific IgG titers, which were similar to the preliminary immunization. Mice receiving the trivalent bioconjugate, all had elevations in serotype specific IgG titers when compared to control as expected, day 49 sera have shown much more elevated IgG tires for serotypes 8 and 14 compared to serotype 9V. Nevertheless, IgG titers against 9V were still significantly higher than the placebo.

Certain embodiments of the present disclosure can be defined in any of the following numbered paragraphs:

1. A glycoconjugate comprising an oligo- or polysaccharide covalently linked to a fusion protein: wherein the fusion protein comprises a ComP protein (ComP) glycosylation fragment; wherein the ComP glycosylation fragment does not contain a cysteine residue corresponding to the conserved cysteine residue at position 71 of ComP₁₁₀₂₆₄ (SEQ ID NO: 1) and/or does not contain a cysteine residue corresponding to the conserved cysteine residue at position 93 of ComP₁₁₀₂₆₄ (SEQ ID NO: 1); wherein the ComP glycosylation fragment is located internally within the fusion protein; and wherein the fusion protein is glycosylated with the oligo- or polysaccharide on the ComP glycosylation fragment at serine residue corresponding to the conserved serine residue at position 82 of ComP₁₁₀₂₆₄ (SEQ ID NO: 1); optionally, wherein the glycoconjugate is immunogenic; optionally, wherein the ComP glycosylation fragment is solvent (or surface)-exposed; optionally, wherein the ComP glycosylation fragment is integrated into a C₁₀ β-turn, β-turn, β-twist, β-loop, U turn, reverse turn, chain reversal, or a hairpin loop of the fusion protein.

2. The glycoconjugate of Paragraph 1, wherein the ComP glycosylation fragment has a length of from 5 to 22 amino acids in length, has a length of from 10 to 22 amino acids in length, has a length of from 11 to 22 amino acids in length, has a length of from 5 to 21 amino acids in length, has a length of from 10 to 21 amino acids in length, or has a length of from 11 to 21 amino acids in length; optionally, wherein the fragment has at least 1, 2, 3, 4, or 5 amino acid residues N-terminal to the serine residue corresponding to the conserved serine residue at position 82 of SEQ ID NO: 1 and/or wherein the fragment has at least 1, 2, 3, 4, or 5 amino acid residues C-terminal to the serine residue corresponding to the conserved serine residue at position 82 of SEQ ID NO: 1.

3. The glycoconjugate of Paragraph 1 or 2, wherein the amino acid sequence of the ComP glycosylation fragment does not extend in the N-terminus direction beyond the amino acid residue corresponding to position 72 of ComP₁₁₀₂₆₄ (SEQ ID NO: 1) and/or does not extend in the C-terminus beyond the amino acid residue corresponding to position 92 of ComP₁₁₀₂₆₄ (SEQ ID NO: 1).

4. The glycoconjugate of any one of Paragraphs 1 to 3, wherein the ComP protein comprises an amino acid sequence that is at least 50%, 60%, 70%, 75%, 80%, 85%, 90%, 95%, 96%, 97%, 98%, 99%, or 100% identical to SEQ ID NO: 9 (ComPΔ28110264) SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT}_H₅₉); optionally, wherein the ComP protein comprises SEQ ID NO: 9 (ComPΔ28₁₁₀₂₆₄), SEQ ID NO: 10 (ComPΔ28_{ADP1}), SEQ ID NO: 11 (ComPΔ28G_{FJ}-2), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59}).

5. The glycoconjugate of any one of Paragraphs 1 to 4, wherein the ComP glycosylation fragment comprises or consists of the amino acid consensus sequence of: X₁GVX₄X₅X₆X₇X₈X₉ASX₁₂X₁₃TX₁₅NVX₁₈X₁₉X₂₀X₂₁ (SEQ ID NO: 17) wherein: X₁ is V, T, A, or I; X₄ is Q, T, E, A, or S; X₅ is E, Q, T, or L; X₆ is I or V; X₇ is S, N, A, or G; X₈ is S or no amino acid; X₉ is G, D, or no amino acid; X₁₂ is N, S, or A; X₁₃ is A, S, or K; X₁₅ is T, S, or K; X₁₈ is A, E, Q, or L; X₁₉ is T, S, or K; X₂₀ is A or S; and X₂₁ is T, Q, A, or V; or a fragment of thereof of at least 5, 6, 7, 8, 9, 10, or 11 amino acids in length comprising the serine (S) residue at position 11 of SEQ ID NO: 17, optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17; or a variant of the amino acid consensus sequence of SEQ ID NO: 17 or the fragment thereof, having one, two, or three amino acid substitutions, additions, and/or deletions, wherein the variant maintains the serine (S) residue at position 11 of SEQ ID NO: 17, optionally, wherein the variant has at least 1, 2, 3, 4, 5, or 6 amino acid residues N-terminal to the serine (S) residue at position 11 of SEQ ID NO: 17 and/or wherein the variant has at least 1, 2, 3, 4, 5, or 6 amino acid residues C-terminal to the serine (S) residue at position 11 of SEQ ID NO: 17; optionally, wherein the ComP glycosylation fragment can be glycosylated when located internally in a fusion protein; and optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

6. The glycoconjugate of any one of Paragraphs 1 to 4, wherein the ComP glycosylation fragment comprises or consists of the amino acid consensus sequence of: X₁GVX₄X₅X₆X₇X₈X₉ASX₁₂X₁₃TX₁₅NVX₁₈X₁₉X₂₀X₂₁ (SEQ ID NO: 17) wherein: X₁ is V, T, A, or I; X₄ is Q, T, E, A, or S; X₅ is E, Q, T, or L; X₆ is I or V; X₇ is S, N, A, or G; X₈ is S or no amino acid; X₉ is G, D, or no amino acid; X₁₂ is N, S, or A; X₁₃ is A, S, or K; X₁₅ is T, S, or K; X₁₈ is A, E, Q, or L; X₁₉ is T, S, or K; X₂₀ is A or S; and X₂₁ is T, Q, A, or V; or a fragment of thereof of at least 5, 6, 7, 8, 9, 10, or 11 amino acids in length comprising the serine (S) residue at position 11 of SEQ ID NO: 17, optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17; optionally, wherein the ComP glycosylation fragment can be glycosylated when located internally in a fusion protein; and optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

7. The glycoconjugate of any one of Paragraphs 1 to 6, wherein the fusion protein comprises a carrier protein selected from the group consisting of Pseudomonas aeruginosa Exotoxin A (EPA), CRM₁₉₇, cholera toxin B subunit, tetanus toxin C fragment, Haemophilus influenzae Protein D, and a fragment or fragments thereof; optionally, wherein the Pseudomonas aeruginosa Exotoxin A (EPA) carrier protein comprises the amino acid sequence of SEQ ID NO: 18, or a fragment or fragments thereof; optionally, wherein the CRM₁₉₇ carrier protein comprises the amino acid sequence of SEQ ID NO: 24, or a fragment or fragments thereof.

8. The glycoconjugate of Paragraph 7, wherein: (i) the ComP glycosylation fragment is inserted between Ala489 and Arg490 relative to the PDB entity 1IKQ of Pseudomonas aeruginosa Exotoxin A (EPA) (SEQ ID NO: 19); (ii) the ComP glycosylation fragment is inserted between Glu548 and Gly549 relative to the PDB entity 1IKQ of Pseudomonas aeruginosa Exotoxin A (EPA) (SEQ ID NO:20); (iii) the ComP glycosylation fragment is inserted between Ala122 and Gly123 relative to the PDB entity 1IKQ of Pseudomonas aeruginosa Exotoxin A (EPA) (SEQ ID NO: 21); (iv) the ComP glycosylation fragment is inserted between Thr355 and Gly356 relative to the PDB entity 1IKQ of Pseudomonas aeruginosa Exotoxin A (EPA) (SEQ ID NO: 22); or (v) the ComP glycosylation fragment is inserted between Lys20 and Asp21 relative to the PDB entity 1IKQ of Pseudomonas aeruginosa Exotoxin A (EPA) (SEQ ID NO: 23).

9. The glycoconjugate of Paragraph 7, wherein: (i) the ComP glycosylation fragment is inserted between Asn481 and Gly482 relative to the PDB entity 4AE0 of CRM₁₉₇ (SEQ ID NO: 25); (ii) the ComP glycosylation fragment is inserted between Asp392 and Gly393 relative to the PDB entity 4AE0 of CRM₁₉₇ (SEQ ID NO: 26); (iii) the ComP glycosylation fragment is inserted between Glu142 and Gly143 relative to the PDB entity 4AE0 of CRM₁₉₇ (SEQ ID NO: 27); (iv) the ComP glycosylation fragment is inserted between Asp129 and Gly130 relative to the PDB entity 4AE0 of CRM₁₉₇ (SEQ ID NO: 28); or (v) the ComP glycosylation fragment is inserted between Asn69 and Glu70 relative to the PDB entity 4AE0 of CRM₁₉₇ (SEQ ID NO: 29).

10. The glycoconjugate of any one of Paragraphs 1 to 9, wherein the fusion protein comprises two or more, three or more, four or more, five or more, six or more, eight or more, ten or more, fifteen or more, or twenty or more ComP glycosylation fragments; optionally, wherein the fusion protein does not comprise more than three, more than five, more than ten, more than fifteen, more than twenty, or more than twenty five ComP glycosylation fragments.

11. The glycoconjugate of any one of Paragraphs 1 to 10, wherein the ComP glycosylation fragments are identical.

12. The glycoconjugate of any one of Paragraphs 1 to 10, wherein the ComP glycosylation fragments differ from each other; optionally, wherein at least three, at least four, or at least five of the ComP glycosylation fragments all differ from each other; optionally, wherein none of the ComP glycosylation fragments are the same.

13. The glycoconjugate of any one of Paragraphs 1 to 12, wherein the oligo- or polysaccharide is derived from a saccharide produced by bacteria from the genus Streptococcus; optionally, wherein the saccharide is a S. pneumoniae, S. agalactiae, or S. suis capsular polysaccharide; optionally, wherein the saccharide is the serotype 8 capsular polysaccharide from S. pneumoniae; optionally, wherein the saccharide is the type Ia, Ib, II, III, IV, V, VI, VII, VIII, or X capsular polysaccharide from S. agalactiae.

14. The glycoconjugate of any one of Paragraphs 1 to 12, wherein the oligo- or polysaccharide is derived from a saccharide produced by the bacteria from the genus Klebsiella; optionally, wherein the saccharide is a K. pneumoniae, K. varricola, K. michinganenis, or K. oxytoca capsular polysaccharide; optionally, wherein the saccharide is a K. pneumoniae, K. varricola, K. michinganenis, or K. oxytoca 0-antigen polysaccharide.

15. The glycoconjugate of any one of Paragraphs 1 to 14, wherein oligo- or polysaccharide comprises glucose at its reducing end.

16. The glycoconjugate of any one of Paragraphs 1 to 15, wherein the glycoconjugate is produced in vivo; optionally, in a bacterial cell; optionally, in Escherichia coli; optionally, in a bacterium from the genus Klebsiella; optionally, wherein the bacterial species is K. pneumoniae, K. varricola, K. michinganenis, or K. oxytoca.

17. The glycoconjugate of any one of Paragraphs 1 to 16, wherein the ComP glycosylation fragment comprises or consists of an amino acid sequence of SEQ ID NO: 32-163, or 164, or a variant thereof having one, two, or three amino acid substitutions, additions, and/or deletions, wherein the variant comprises the serine residue corresponding to the conserved serine residue at position 82 of SEQ ID NO: 1; optionally, wherein the ComP glycosylation fragment can be glycosylated when located internally in a fusion protein; and optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

18. The glycoconjugate of Paragraph 17, wherein the ComP glycosylation fragment comprises or consists of an amino acid sequence of:

iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATTNVASA;
iGTccΔ2-1
(SEQ ID NO: 56)
GVTQIASGASAATTNVASAQ;
iGTccΔ2-2
(SEQ ID NO: 57)
GVTQIASGASAATTNVASA;
iGTccΔ2-3
(SEQ ID NO: 58)
GVTQIASGASAATTNVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN,

19. The glycoconjugate of Paragraph 17, wherein the ComP glycosylation fragment comprises or consists of an amino acid sequence of SEQ ID NO: 32-163, or 164, optionally, wherein the ComP glycosylation fragment can be glycosylated when located internally in a fusion protein; and optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

20. The glycoconjugate of Paragraph 19, wherein the ComP glycosylation fragment comprises or consists of an amino acid sequence of:

iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATTNVASA;
iGTccΔ2-1
(SEQ ID NO: 56)
GVTQIASGASAATTNVASAQ;
iGTccΔ2-2
(SEQ ID NO: 57)
GVTQIASGASAATTNVASA;
iGTccΔ2-3
(SEQ ID NO: 58)
GVTQIASGASAATTNVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN.

21. The glycoconjugate of any one of Paragraphs 1 to 20, wherein the bioconjugate is a conjugate vaccine; optionally, wherein the conjugate vaccine is a vaccine against Streptococcus pneumoniae serotype 8.

22. The glycoconjugate of Paragraph 21, wherein when the conjugate vaccine induces an immune response when administered to a subject.

23. The glycoconjugate of Paragraph 22, wherein the immune response elicits long term memory (memory B and T cells), is an antibody response, and is optionally a serotype-specific antibody response.

24. The glycoconjugate of Paragraph 23, wherein the antibody response is an IgG or IgM response.

25. The glycoconjugate of Paragraph 24, wherein the antibody response is an IgG response; optionally an IgG1 response.

26. The glycoconjugate of any one of Paragraphs 21 to 25, wherein the conjugate vaccine generates immunological memory in a subject administered the vaccine.

27. A ComP glycosylation fragment comprising or consisting of an isolated fragment of a ComP protein, wherein the ComP glycosylation fragment does not contain a cysteine residue corresponding to the conserved cysteine residue at position 71 of ComP₁₁₀₂₆₄ (SEQ ID NO: 1) and/or does not contain a cysteine residue corresponding to the conserved cysteine residue at position 93 of ComP₁₁₀₂₆₄ (SEQ ID NO: 1); and wherein the ComP glycosylation fragment comprises the serine residue corresponding to the conserved serine residue at position 82 of ComP₁₁₀₂₆₄ (SEQ ID NO: 1); optionally, wherein the ComP glycosylation fragment is immunogenic.

28. The ComP glycosylation fragment of Paragraph 27, wherein the ComP glycosylation fragment has a length of from 5 to 22 amino acids in length, has a length of from 10 to 22 amino acids in length, has a length of from 11 to 22 amino acids in length, has a length of from 5 to 21 amino acids in length, has a length of from 10 to 21 amino acids in length, or has a length of from 11 to 21 amino acids in length; optionally, wherein the fragment has at least 1, 2, 3, 4, 5, or 6 amino acid residues N-terminal to the serine residue corresponding to the conserved serine residue at position 82 of SEQ ID NO: 1 and/or wherein the fragment has at least 1, 2, 3, 4, 5, or 6 amino acid residues C-terminal to the serine residue corresponding to the conserved serine residue at position 82 of SEQ ID NO: 1.

29. The ComP glycosylation fragment of Paragraph 27 or 28, wherein the amino acid sequence of the ComP glycosylation fragment does not extend in the N-terminus direction beyond the amino acid residue corresponding to position 72 of ComP₁₁₀₂₆₄ (SEQ ID NO: 1) and/or does not extend in the C-terminus beyond the amino acid residue corresponding to position 92 of ComP₁₁₀₂₆₄ (SEQ ID NO: 1).

30. The ComP glycosylation fragment of any one of Paragraphs 27 to 29, wherein the ComP protein comprises an amino acid sequence that is at least 50%, 60%, 70%, 75%, 80%, 85%, 90%, 95%, 96%, 97%, 98%, 99%, or 100% identical to SEQ ID NO: 9 (ComPΔ28₁₁₀₂₆₄), SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59}); optionally, wherein the ComP protein comprises SEQ ID NO: 9 (ComPΔ28₁₁₀₂₆₄), SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59}).

31. The ComP glycosylation fragment of any one of Paragraphs 27 to 30, wherein the ComP glycosylation fragment comprises or consists of the amino acid consensus sequence of: X₁GVX₄X₅X₆X₇X₈X₉ASX₁₂X₁₃TX₁₅NVX₁₈X₁₉X₂₀X₂₁ (SEQ ID NO: 17) wherein: X₁ is V, T, A, or I; X₄ is Q, T, E, A, or S; X₅ is E, Q, T, or L; X₆ is I or V; X₇ is S, N, A, or G; X₈ is S or no amino acid; X₉ is G, D, or no amino acid; X₁₂ is N, S, or A; X₁₃ is A, S, or K; X₁₅ is T, S, or K; X₁₈ is A, E, Q, or L; X₁₉ is T, S, or K; X₂₀ is A or S; and X₂₁ is T, Q, A, or V; or a fragment of thereof of at least 5, 6, 7, 8, 9, 10, or 11 amino acids in length comprising the serine (S) residue at position 11 of SEQ ID NO: 17, optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17; or a variant of the amino acid consensus sequence of SEQ ID NO: 17 or the fragment thereof, having one, two, three, four, five, six, or seven amino acid substitutions, additions, and/or deletions, wherein the variant maintains the serine (S) residue at position 11 of SEQ ID NO: 17, optionally, wherein the variant has at least 1, 2, 3, 4, 5, or 6 amino acid residues N-terminal to the serine (S) residue at position 11 of SEQ ID NO: 17 and/or wherein the variant has at least 1, 2, 3, 4, 5, or 6 amino acid residues C-terminal to the serine (S) residue at position 11 of SEQ ID NO: 17; optionally, wherein the ComP glycosylation fragment can be glycosylated when located internally in a fusion protein; and optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

32. The ComP glycosylation fragment of any one of Paragraphs 27 to 30, wherein the ComP glycosylation fragment comprises or consists of the amino acid consensus sequence of: X₁GVX₄X₅X₆X₇X₈X₉ASX₁₂X₁₃TX₁₅NVX₁₈X₁₉X₂₀X₂₁ (SEQ ID NO: 17) wherein: X₁ is V, T, A, or I; X₄ is Q, T, E, A, or S; X₅ is E, Q, T, or L; X₆ is I or V; X₇ is S, N, A, or G; X₈ is S or no amino acid; X₉ is G, D, or no amino acid; X₁₂ is N, S, or A; X₁₃ is A, S, or K; X₁₅ is T, S, or K; X₁₈ is A, E, Q, or L; X₁₉ is T, S, or K; X₂₀ is A or S; and X₂₁ is T, Q, A, or V; or a fragment of thereof of at least 5, 6, 7, 8, 9, 10, or 11 amino acids in length comprising the serine (S) residue at position 11 of SEQ ID NO: 17, optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17; optionally, wherein the ComP glycosylation fragment can be glycosylated when located internally in a fusion protein; and optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

33. The ComP glycosylation fragment of Paragraph 27, wherein the ComP glycosylation fragment comprises or consists of an amino acid sequence of SEQ ID NO: 32-163, or 164, or a variant thereof having one, two, or three amino acid substitutions, additions, and/or deletions, wherein the variant comprises the serine residue corresponding to the conserved serine (S) residue at position 82 of SEQ ID NO: 1; optionally, wherein the ComP glycosylation fragment can be glycosylated when located internally in a fusion protein; and optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

34. The ComP glycosylation fragment of Paragraph 33, wherein the ComP glycosylation fragment comprises or consists of an amino acid sequence of:

iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATTNVASA;
iGTccΔ2-1
(SEQ ID NO: 56)
GVTQIASGASAATTNVASAQ;
iGTccΔ2-2
(SEQ ID NO: 57)
GVTQIASGASAATTNVASA;
iGTccΔ2-3
(SEQ ID NO: 58)
GVTQIASGASAATTNVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN,

or the variant thereof.

35. The ComP glycosylation fragment of Paragraph 33, wherein the ComP glycosylation fragment comprises or consists of an amino acid sequence of SEQ ID NO: 32-163, or 164, optionally, wherein the ComP glycosylation fragment can be glycosylated when located internally in a fusion protein; and optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

36. The ComP glycosylation fragment of Paragraph 35, wherein the ComP glycosylation fragment comprises or consists of an amino acid sequence of:

iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATTNVASA;
iGTccΔ2-1
(SEQ ID NO: 56)
GVTQIASGASAATTNVASAQ;
iGTccΔ2-2
(SEQ ID NO: 57)
GVTQIASGASAATTNVASA;
iGTccΔ2-3
(SEQ ID NO: 58)
GVTQIASGASAATTNVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN.

37. A fusion protein comprising the ComP glycosylation fragment of any of Paragraphs 27 to 36, wherein the ComP glycosylation fragment is located internally within the fusion protein; optionally, wherein the fusion protein is glycosylated by an oligo- or polysaccharide at a serine residue on the glycosylation fragment corresponding to the serine ComP glycosylation fragment residue at position 82 of SEQ ID NO: 1 (ComP₁₁₀₂₆₄).

38. The fusion protein of Paragraph 37, wherein the oligo- or polysaccharide is derived from a saccharide produced by bacteria from the genus Streptococcus; optionally, wherein the saccharide is a S. pneumoniae, S. agalactiae, or S. suis capsular polysaccharide; optionally, wherein the saccharide is the serotype 8 capsular polysaccharide from S. pneumoniae; optionally, wherein the saccharide is the type Ia, Ib, II, III, IV, V, VI, VII, VIII, or X capsular polysaccharide from S. agalactiae.

39. The fusion protein of Paragraph 37, wherein the oligo- or polysaccharide is derived from a saccharide produced by the bacteria from the genus Klebsiella; optionally, wherein the saccharide is a K. pneumoniae, K. varricola, K. michinganenis, or K. oxytoca capsular polysaccharide; optionally, wherein the saccharide is a K. pneumoniae, K. varricola, K. michinganenis, or K. oxytoca O-antigen polysaccharide.

40. The fusion protein of any one of Paragraphs 37 to 39, wherein oligo- or polysaccharide comprises glucose at its reducing end.

41. The fusion protein of any one of Paragraphs 37 to 40, wherein the glycosylated fusion protein is produced in vivo; optionally, in a bacterial cell; optionally, in Escherichia coli; optionally, in a bacterium from the genus Klebsiella; optionally, wherein the bacterial species is K. pneumoniae, K. varricola, K. michinganenis, or K. oxytoca.

42. The fusion protein of any one of Paragraphs 37 to 41, wherein the fusion protein comprises a carrier protein selected from the group consisting of Pseudomonas aeruginosa Exotoxin A (EPA), CRM₁₉₇, cholera toxin B subunit, tetanus toxin C fragment, Haemophilus influenzae Protein D, and a fragment or fragments thereof; optionally, wherein the Pseudomonas aeruginosa Exotoxin A (EPA) carrier protein comprises the amino acid sequence of SEQ ID NO: 18, or a fragment or fragments thereof; optionally, wherein the CRM₁₉₇ carrier protein comprises the amino acid sequence of SEQ ID NO: 24, or a fragment or fragments thereof.

43. The fusion protein of Paragraph 42, wherein: (i) the ComP glycosylation fragment is inserted between Ala489 and Arg490 relative to the PDB entity 1IKQ of Pseudomonas aeruginosa Exotoxin A (EPA) (SEQ ID NO: 19); (ii) the ComP glycosylation fragment is inserted between Glu548 and Gly549 relative to the PDB entity 1IKQ of Pseudomonas aeruginosa Exotoxin A (EPA) (SEQ ID NO: 20); (iii) the ComP glycosylation fragment is inserted between Ala122 and Gly123 relative to the PDB entity 1IKQ of Pseudomonas aeruginosa Exotoxin A (EPA) (SEQ ID NO: 21); (iv) the ComP glycosylation fragment is inserted between Thr355 and Gly356 relative to the PDB entity 1IKQ of Pseudomonas aeruginosa Exotoxin A (EPA) (SEQ ID NO: 22); or (v) the ComP glycosylation fragment is inserted between Lys20 and Asp21 relative to the PDB entity 1IKQ of Pseudomonas aeruginosa Exotoxin A (EPA) (SEQ ID NO: 23).

44. The fusion protein of Paragraph 42, wherein: (i) the ComP glycosylation fragment is inserted between Asn481 and Gly482 relative to the PDB entity 4AE0 of CRM₁₉₇ (SEQ ID NO: 25); (ii) the ComP glycosylation fragment is inserted between Asp392 and Gly393 relative to the PDB entity 4AE0 of CRM₁₉₇ (SEQ ID NO: 26); (iii) the ComP glycosylation fragment is inserted between Glu142 and Gly143 relative to the PDB entity 4AE0 of CRM₁₉₇ (SEQ ID NO: 27); (iv) the ComP glycosylation fragment is inserted between Asp129 and Gly130 relative to the PDB entity 4AE0 of CRM₁₉₇ (SEQ ID NO: 28); or (v) the ComP glycosylation fragment is inserted between Asn69 and Glu70 relative to the PDB entity 4AE0 of CRM₁₉₇ (SEQ ID NO: 29).

45. The fusion protein of any one of Paragraph s 37 to 44, wherein the fusion protein comprises two or more, three or more, four or more, five or more, six or more, eight or more, ten or more, fifteen or more, or twenty or more ComP glycosylation fragments; optionally, wherein the fusion protein does not comprise more than three, more than five, more than ten, more than fifteen, more than twenty, or more than twenty five ComP glycosylation fragments.

46. The fusion protein of any one of Paragraphs 37 to 45, wherein the ComP glycosylation fragments are identical.

47. The fusion protein of any one of Paragraphs 37 to 45, wherein the ComP glycosylation fragments differ from each other; optionally, wherein at least three, at least four, or at least five of the ComP glycosylation fragments all differ from each other; optionally, wherein none of the ComP glycosylation fragments are the same.

48. A method of in vivo conjugation of an oligo- or polysaccharide to an acceptor polypeptide, the method comprising covalently linking the oligo- or polysaccharide to the acceptor polypeptide with a PglS oligosaccharyltransferase (OTase), wherein the acceptor polypeptide comprises the ComP glycosylation fragment of any one of Paragraphs 27 to 36.

49. The method of Paragraph 48, wherein the PglS OTase is PglS₁₁₀₂₆₄ (SEQ ID NO: 165), PglS_{ADP1} (SEQ ID NO: 166), PglS_{GFJ-2} (SEQ ID NO: 167), PglS₅₀ᵥ₁ (SEQ ID NO: 168), PglS₄₄₆₆ (SEQ ID NO: 169), PglS_{SFC} (SEQ ID NO: 170), Pgl_{SP5312} (SEQ ID NO: 171), or PglS_{ANT_H59} (SEQ ID NO: 172).

50. The method of Paragraph 48 or 49, wherein the oligo- or polysaccharide is linked to the ComP glycosylation fragment at a serine (S) residue corresponding to the serine residue at position 82 of SEQ ID NO: 1 (ComP₁₁₀₂₆₄).

51. The method of any one of Paragraphs 48 to 50, wherein the in vivo conjugation occurs in a host cell.

52. The method of Paragraph 51, wherein the host cell is a bacterial cell; optionally, in Escherichia coli; optionally, in a bacterium from the genus Klebsiella; optionally, wherein the bacterial species is K. pneumoniae, K. varricola, K. michinganenis, or K. oxytoca.

53. The method of Paragraph 51 of 52, comprising culturing a host cell that comprises: (a) a genetic cluster encoding for the proteins required to synthesize the oligo- or polysaccharide; (b) a PglS OTase; and (3) the acceptor polypeptide.

54. The method of any one of Paragraphs 48 to 53, wherein production of the oligo- or polysaccharide is enhanced by the K. pneumoniae transcriptional activator rmpA (K. pneumoniae NTUH K-2044) or a homolog of the K. pneumoniae transcriptional activator rmpA (K. pneumoniae NTUH K-2044).

55. The method of any one of Paragraphs 48 to 54, wherein the method produces a conjugate vaccine.

56. A host cell comprising (a) a genetic cluster encoding for the proteins required to synthesize an oligo- or polysaccharide; (b) a PglS OTase; and (3) an acceptor polypeptide comprising the ComP glycosylation fragment of any one of Paragraphs 27 to 36.

57. The host cell of Paragraph 56, wherein the acceptor polypeptide is a fusion protein.

58. The host cell of Paragraph 56 or 57, wherein the host cell comprises a nucleic acid encoding the PglS OTase.

59. The host cell of any one of Paragraphs 56 to 58, wherein the host cell comprises a nucleic acid encoding the acceptor polypeptide.

60. An isolated nucleic acid encoding the ComP glycosylation fragment of any one of Paragraphs 27 to 36 and/or the fusion protein of any one of Paragraphs 37 to 47.

61. The isolated nucleic acid of Paragraph 60, wherein the nucleic acid is a vector.

62. A host cell comprising the isolated nucleic acid of Paragraph 60 or 61.

63. A composition comprising the conjugate vaccine of any one of Paragraphs 21 to 26 or the fusion protein of any one of Paragraphs 37 to 47, and an adjuvant.

64. A method of inducing a host immune response against a bacterial pathogen, the method comprising administering to a subject in need of the immune response an effective amount of the conjugate vaccine of any one of Paragraphs 21 to 26, the fusion protein of any one of Paragraphs 37 to 47, or the composition of Paragraph 63.

65. The method of Paragraph 64, wherein the immune response is an antibody response.

66. The method of Paragraph 64, wherein the immune response is selected from the group consisting of an innate response, an adaptive response, a humoral response, an antibody response, cell mediated response, a B cell response, a T cell response, cytokine upregulation or downregulation, immune system cross-talk, and a combination of two or more of said immune responses.

67. The method of Paragraph 64, wherein the immune response is selected from the group consisting of an innate response, a humoral response, an antibody response, a T cell response, and a combination of two or more of said immune responses.

68. A method of preventing or treating a bacterial disease and/or infection in a subject comprising administering to a subject in need thereof the conjugate vaccine of any one of Paragraphs 21 to 26, the fusion protein of any one of Paragraphs 37 to 47, or the composition of Paragraph 63.

69. The method of Paragraph 68, wherein the infection is a localized or systemic infection of skin, soft tissue, blood, or an organ, or is auto-immune in nature.

70. The method of Paragraph 69, wherein the disease is pneumonia.

71. The method of Paragraph 69, wherein the infection is a systemic infection and/or an infection of the blood.

72. The method of any one of Paragraphs 68 to 71, wherein the subject is a human.

73. The method of any one of Paragraph s 68 to 72, wherein the composition is administered via intramuscular injection, intradermal injection, intraperitoneal injection, subcutaneous injection, intravenous injection, oral administration, mucosal administration, intranasal administration, or pulmonary administration.

74. A method of producing a pneumococcal conjugate vaccine against pneumococcal infection, the method comprising: (a) isolating the glycoconjugate of any one of Paragraphs 1 to 26 or a glycosylated fusion protein of any one of Paragraphs 37 to 47; and (b) combining the isolated glycoconjugate or isolated glycosylated fusion protein with an adjuvant.

75. The glycoconjugate, glycosylated fusion protein, or conjugate vaccine of any of the above paragraphs for use in inducing a host immune response against a bacterial pathogen and/or preventing or treating a bacterial disease and/or infection in a subject.

The breadth and scope of the present disclosure should not be limited by any of the above-described exemplary embodiments, but should be defined only in accordance with the appended claims and their equivalents.

### Sequences

PDB entity 1IKQ of Pseudomonas aeruginosa Exotoxin A (EPA) SEQ ID NO: 18

SEQ ID NO: 18
AEEAFDLWNECAKACVLDLKDGVRSSRMSVDPAIADTNGQGVLHYSMVL
EGGNDALKLAIDNALSITSDGLTIRLEGGVEPNKPVRYSYTRQARGSWS
LNWLVPIGHEKPSNIKVFIHELNAGNQLSHMSPIYTIEMGDELLAKLAR
DATFFVRAHESNEMQPTLAISHAGVSVVMAQAQPRREKRWSEWASGKVL
CLLDPLDGVYNYLAQQRCNLDDTWEGKIYRVLAGNPAKHDLDIKPTVIS
HRLHFPEGGSLAALTAHQACHLPLETFTRHRQPRGWEQLEQCGYPVQRL
VALYLAARLSWNQVDQVIRNALASPGSGGDLGEAIREQPEQARLALTLA
AAESERFVRQGTGNDEAGAASADVVSLTCPVAAGECAGPADSGDALLER
NYPTGAEFLGDGGDISFSTRGTQNWTVERLLQAHRQLEERGYVFVGYHG
TFLEAAQSIVFGGVRARSQDLDAIWRGFYIAGDPALAYGYAQDQEPDAR
GRIRNGALLRVYVPRSSLPGFYRTGLTLAAPEAAGEVERLIGHPLPLRL
DAITGPEEEGGRLTILGWPLAERTVVIPSAIPTDPRNVGGDLDPSSIPD
KEQAISALPDYASQPGKPPREDLK

ssDsbA-EPA-iGT-SITE1-6×His—DsbA signal sequence and hexahistidine tag underlined, the ComP glycosylation fragment insertion site is located between the underlined, bolded amino acid residues

SEQ ID NO: 19
MKKIWLALAGLVLAFSASAAEEAFDLWNECAKACVLDLKDGVRSSRMSV
DPALADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLETFTRH
RQPRGWEQLEQCGYPVQRLVALYLAARLSWNQVDQVIRNALASPGSGGD
LGEAIREQPEQARLALTLAAAESERFVRQGTGNDEAGAASADVVSLTCP
VAAGECAGPADSGDALLERNYPTGAEFLGDGGDISFSTRGTQNWTVERL
LQAHRQLEERGYVFVGYHGTFLEAAQSIVFGGVRARSQDLDAIWRGFYI
AGDPALAYGYAQDQEPDARGRIRNGALLRVYVPRSSLPGFYRTGLTLAA
PEAAGEVERLIGHPLPLRLDAITGPEEEGGRLTILGWPLAERTVVIPSA
IPTDPRNVGGDLDPSSIPDKEQAISALPDYASQPGKPPREDLKHHHHHH

ssDsbA-EPA-iGT-SITE2-6×His—DsbA signal sequence and hexahistidine tag underlined, ComP glycosylation fragment insertion site is located between the underlined, bolded amino acid residues

SEQ ID NO: 20
MKKIWLALAGLVLAFSASAAEEAFDLWNECAKACVLDLKDGVRSSRMSVDPAIAD
TNGQGVLHYSMVLEGGNDALKLAIDNALSITSDGLTIRLEGGVEPNKPVRYSYTRQA
RGSWSLNWLVPIGHEKPSNIKVFIHELNAGNQLSHMSPIYTIEMGDELLAKLARDATF
FVRAHESNEMQPTLAISHAGVSVVMAQAQPRREKRWSEWASGKVLCLLDPLDGVY
NYLAQQRCNLDDTWEGKIYRVLAGNPAKHDLDIKPTVISHRLHFPEGGSLAALTAH
QACHLPLETFTRHRQPRGWEQLEQCGYPVQRLVALYLAARLSWNQVDQVIRNALAS
PGSGGDLGEAIREQPEQARLALTLAAAESERFVRQGTGNDEAGAASADVVSLTCPVA
AGECAGPADSGDALLERNYPTGAEFLGDGGDISFSTRGTQNWTVERLLQAHRQLEE
RGYVFVGYHGTFLEAAQSIVFGGVRARSQDLDAIWRGFYIAGDPALAYGYAQDQEP
DARGRIRNGALLRVYVPRSSLPGFYRTGLTLAAPEAAGEVERLIGHPLPLRLDAITGP
EEEGGRLTILGWPLAERTVVIPSAIPTDPRNVGGDLDPSSIPDKEQAISALPDYASQPG
KPPREDLKHHHHHH

ssDsbA-EPA-iGT-SITE3-6×His—DsbA signal sequence and hexahistidine tag underlined, ComP glycosylation fragment insertion site is located between the underlined, bolded amino acid residues

SEQ ID NO: 21
MKKIWLALAGLVLAFSASAAEEAFDLWNECAKACVLDLKDGVRSSRMSVDPAIAD
TNGQGVLHYSMVLEGGNDALKLAIDNALSITSDGLTIRLEGGVEPNKPVRYSYTRQA
RGSWSLNWLVPIGHEKPSNIKVFIHELNAGNQLSHMSPIYTIEMGDELLAKLARDATF
FVRAHESNEMQPTLAISHAGVSVVMAQAQPRREKRWSEWASGKVLCLLDPLDGVY
NYLAQQRCNLDDTWEGKIYRVLAGNPAKHDLDIKPTVISHRLHFPEGGSLAALTAH
QACHLPLETFTRHRQPRGWEQLEQCGYPVQRLVALYLAARLSWNQVDQVIRNALAS
PGSGGDLGEAIREQPEQARLALTLAAAESERFVRQGTGNDEAGAASADVVSLTCPVA
AGECAGPADSGDALLERNYPTGAEFLGDGGDISFSTRGTQNWTVERLLQAHRQLEE
RGYVFVGYHGTFLEAAQSIVFGGVRARSQDLDAIWRGFYIAGDPALAYGYAQDQEP
DARGRIRNGALLRVYVPRSSLPGFYRTGLTLAAPEAAGEVERLIGHPLPLRLDAITGP
EEEGGRLTILGWPLAERTVVIPSAIPTDPRNVGGDLDPSSIPDKEQAISALPDYASQPG
KPPREDLKHHHHHH

ssDsbA-EPA-iGT-SITE4-6×His—DsbA signal sequence and hexahistidine tag underlined, ComP glycosylation fragment insertion site located between the underlined, bolded amino acid residues

SEQ ID NO: 22
MKKIWLALAGLVLAFSASAAEEAFDLWNECAKACVLDLKDGVRSSRMSVDPAIAD
TNGQGVLHYSMVLEGGNDALKLAIDNALSITSDGLTIRLEGGVEPNKPVRYSYTRQA
RGSWSLNWLVPIGHEKPSNIKVFIHELNAGNQLSHMSPIYTIEMGDELLAKLARDATF
FVRAHESNEMQPTLAISHAGVSVVMAQAQPRREKRWSEWASGKVLCLLDPLDGVY
NYLAQQRCNLDDTWEGKIYRVLAGNPAKHDLDIKPTVISHRLHFPEGGSLAALTAH
QACHLPLETFTRHRQPRGWEQLEQCGYPVQRLVALYLAARLSWNQVDQVIRNALAS
PGSGGDLGEAIREQPEQARLALTLAAAESERFVRQGTGNDEAGAASADVVSLTCPV
AAGECAGPADSGDALLERNYPTGAEFLGDGGDISFSTRGTQNWTVERLLQAHRQLE
ERGYVFVGYHGTFLEAAQSIVFGGVRARSQDLDAIWRGFYIAGDPALAYGYAQDQE
PDARGRIRNGALLRVYVPRSSLPGFYRTGLTLAAPEAAGEVERLIGHPLPLRLDAITG
PEEEGGRLTILGWPLAERTVVIPSAIPTDPRNVGGDLDPSSIPDKEQAISALPDYASQP
GKPPREDLKHHHHHH

ssDsbA-EPA-iGT-SITE5-6×His—DsbA signal sequence and hexahistidine tag underlined, ComP glycosylation fragment insertion site located between the underlined, bolded amino acid residues

SEQ ID NO: 23
MKKIWLALAGLVLAFSASAAEEAFDLWNECAKACVLDLKDGVRSSRMSVDPAIAD
TNGQGVLHYSMVLEGGNDALKLAIDNALSITSDGLTIRLEGGVEPNKPVRYSYTRQA
RGSWSLNWLVPIGHEKPSNIKVFIHELNAGNQLSHMSPIYTIEMGDELLAKLARDATF
FVRAHESNEMQPTLAISHAGVSVVMAQAQPRREKRWSEWASGKVLCLLDPLDGVY
NYLAQQRCNLDDTWEGKIYRVLAGNPAKHDLDIKPTVISHRLHFPEGGSLAALTAH
QACHLPLETFTRHRQPRGWEQLEQCGYPVQRLVALYLAARLSWNQVDQVIRNALAS
PGSGGDLGEAIREQPEQARLALTLAAAESERFVRQGTGNDEAGAASADVVSLTCPVA
AGECAGPADSGDALLERNYPTGAEFLGDGGDISFSTRGTQNWTVERLLQAHRQLEE
RGYVFVGYHGTFLEAAQSIVFGGVRARSQDLDAIWRGFYIAGDPALAYGYAQDQEP
DARGRIRNGALLRVYVPRSSLPGFYRTGLTLAAPEAAGEVERLIGHPLPLRLDAITGP
EEEGGRLTILGWPLAERTVVIPSAIPTDPRNVGGDLDPSSIPDKEQAISALPDYASQPG
KPPREDLKHHHHHH

PDB entity 4AE0 of CRM197

SEQ ID NO: 24
GADDVVDSSKSFVMENFSSYHGTKPGYVDSIQKGIQKPKSGTQGNYDDDWKEFYST
DNKYDAAGYSVDNENPLSGKAGGVVKVTYPGLTKVLALKVDNAETIKKELGLSLTE
PLMEQVGTEEFIKRFGDGASRVVLSLPFAEGSSSVEYINNWEQAKALSVELEINFETR
GKRGQDAMYEYMAQACAGNRVRRSVGSSLSCINLDWDVIRDKTKTKIESLKEHGPI
KNKMSESPNKTVSEEKAKQYLEEFHQTALEHPELSELKTVTGTNPVFAGANYAAWA
VNVAQVIDSETADNLEKTTAALSILPGIGSVMGIADGAVHHNTEEIVAQSIALSSLMV
AQAIPLVGELVDIGFAAYNFVESIINLFQVVHNSYNRPAYSPGHKTQPFLHDGYAVS
WNTVEDSIIRTGFQGESGHDIKITAENTPLPIAGVLLPTIPGKLDVNKSKTHISVNGRKI
RMRCRAIDGDVTFCRPKSPVYVGNGVHANLHVAFHRSSSEKIHSNEISSDSIGVLGYQ
KTVDHTKVNSKLSLFFEIKS

ssFlgI-CRM197-iGT-SITE1-6×His—FlgI signal sequence and hexahistidine tag underlined, ComP glycosylation fragment insertion site located between the underlined, bolded amino acid residues

SEQ ID NO: 25
MIKFLSALILLLVTTAAQAGADDVVDSSKSFVMENFSSYHGTKPGYVDSIQKGIQKP
KSGTQGNYDDDWKEFYSTDNKYDAAGYSVDNENPLSGKAGGVVKVTYPGLTKVL
ALKVDNAETIKKELGLSLTEPLMEQVGTEEFIKRFGDGASRVVLSLPFAEGSSSVEYI
NNWEQAKALSVELEINFETRGKRGQDAMYEYMAQACAGNRVRRSVGSSLSCINLD
WDVIRDKTKTKIESLKEHGPIKNKMSESPNKTVSEEKAKQYLEEFHQTALEHPELSEL
KTVTGTNPVFAGANYAAWAVNVAQVIDSETADNLEKTTAALSILPGIGSVMGIADG
AVHHNTEEIVAQSIALSSLMVAQAIPLVGELVDIGFAAYNFVESIINLFQVVHNSYNRP
AYSPGHKTQPFLHDGYAVSWNTVEDSIIRTGFQGESGHDIKITAENTPLPIAGVLLPTI
PGKLDVNKSKTHISVNGRKIRMRCRAIDGDVTFCRPKSPVYVGNGVHANLHVAFHR
SSSEKIHSNEISSDSIGVLGYQKTVDHTKVNSKLSLFFEIKSHHHHHH

ssFlgI-CRM197-iGT-SITE2-6×His—FlgI signal sequence and hexahistidine tag underlined, ComP glycosylation fragment insertion site located between the underlined, bolded amino acid residues

SEQ ID NO: 26
MIKFLSALILLLVTTAAQAGADDVVDSSKSFVMENFSSYHGTKPGYVDSIQKGIQKP
KSGTQGNYDDDWKEFYSTDNKYDAAGYSVDNENPLSGKAGGVVKVTYPGLTKVL
ALKVDNAETIKKELGLSLTEPLMEQVGTEEFIKRFGDGASRVVLSLPFAEGSSSVEYI
NNWEQAKALSVELEINFETRGKRGQDAMYEYMAQACAGNRVRRSVGSSLSCINLD
WDVIRDKTKTKIESLKEHGPIKNKMSESPNKTVSEEKAKQYLEEFHQTALEHPELSEL
KTVTGTNPVFAGANYAAWAVNVAQVIDSETADNLEKTTAALSILPGIGSVMGIADG
AVHHNTEEIVAQSIALSSLMVAQAIPLVGELVDIGFAAYNFVESIINLFQVVHNSYNRP
AYSPGHKTQPFLHDGYAVSWNTVEDSIIRTGFQGESGHDIKITAENTPLPIAGVLLPTI
PGKLDVNKSKTHISVNGRKIRMRCRAIDGDVTFCRPKSPVYVGNGVHANLHVAFHR
SSSEKIHSNEISSDSIGVLGYQKTVDHTKVNSKLSLFFEIKSHHHHHH

ssFlgI-CRM197-iGT-SITE3-6×His—FlgI signal sequence and hexahistidine tag underlined, ComP glycosylation fragment insertion site located between the underlined, bolded amino acid residues

SEQ ID NO: 27
MIKFLSALILLLVTTAAQAGADDVVDSSKSFVMENFSSYHGTKPGYVDSIQKGIQKP
KSGTQGNYDDDWKEFYSTDNKYDAAGYSVDNENPLSGKAGGVVKVTYPGLTKVL
ALKVDNAETIKKELGLSLTEPLMEQVGTEEFIKRFGDGASRVVLSLPFAEGSSSVEYI
NNWEQAKALSVELEINFETRGKRGQDAMYEYMAQACAGNRVRRSVGSSLSCINLD
WDVIRDKTKTKIESLKEHGPIKNKMSESPNKTVSEEKAKQYLEEFHQTALEHPELSEL
KTVTGTNPVFAGANYAAWAVNVAQVIDSETADNLEKTTAALSILPGIGSVMGIADG
AVHHNTEEIVAQSIALSSLMVAQAIPLVGELVDIGFAAYNFVESIINLFQVVHNSYNRP
AYSPGHKTQPFLHDGYAVSWNTVEDSIIRTGFQGESGHDIKITAENTPLPIAGVLLPTI
PGKLDVNKSKTHISVNGRKIRMRCRAIDGDVTFCRPKSPVYVGNGVHANLHVAFHR
SSSEKIHSNEISSDSIGVLGYQKTVDHTKVNSKLSLFFEIKSHHHHHH

ssFlgI-CRM197-iGT-SITE4-6×His—FlgI signal sequence and hexahistidine tag underlined, ComP glycosylation fragment insertion site located between the underlined, bolded amino acid residues

SEQ ID NO: 28
MIKFLSALILLLVTTAAQAGADDVVDSSKSFVMENFSSYHGTKPGYVDSIQKGIQKP
KSGTQGNYDDDWKEFYSTDNKYDAAGYSVDNENPLSGKAGGVVKVTYPGLTKVL
ALKVDNAETIKKELGLSLTEPLMEQVGTEEFIKRFGDGASRVVLSLPFAEGSSSVEYI
NNWEQAKALSVELEINFETRGKRGQDAMYEYMAQACAGNRVRRSVGSSLSCINLD
WDVIRDKTKTKIESLKEHGPIKNKMSESPNKTVSEEKAKQYLEEFHQTALEHPELSEL
KTVTGTNPVFAGANYAAWAVNVAQVIDSETADNLEKTTAALSILPGIGSVMGIADG
AVHHNTEEIVAQSIALSSLMVAQAIPLVGELVDIGFAAYNFVESIINLFQVVHNSYNRP
AYSPGHKTQPFLHDGYAVSWNTVEDSIIRTGFQGESGHDIKITAENTPLPIAGVLLPTI
PGKLDVNKSKTHISVNGRKIRMRCRAIDGDVTFCRPKSPVYVGNGVHANLHVAFHR
SSSEKIHSNEISSDSIGVLGYQKTVDHTKVNSKLSLFFEIKSHHHHHH

ssFlgI-CRM197-iGT-SITE5-6×His—FlgI signal sequence and hexahistidine tag underlined, ComP glycosylation fragment insertion site located between the underlined, bolded amino acid residues

SEQ ID NO: 29
MIKFLSALILLLVTTAAQAGADDVVDSSKSFVMENFSSYHGTKPGYVDSIQKGIQKP
KSGTQGNYDDDWKEFYSTDNKYDAAGYSVDNENPLSGKAGGVVKVTYPGLTKVL
ALKVDNAETIKKELGLSLTEPLMEQVGTEEFIKRFGDGASRVVLSLPFAEGSSSVEYI
NNWEQAKALSVELEINFETRGKRGQDAMYEYMAQACAGNRVRRSVGSSLSCINLD
WDVIRDKTKTKIESLKEHGPIKNKMSESPNKTVSEEKAKQYLEEFHQTALEHPELSEL
KTVTGTNPVFAGANYAAWAVNVAQVIDSETADNLEKTTAALSILPGIGSVMGIADG
AVHHNTEEIVAQSIALSSLMVAQAIPLVGELVDIGFAAYNFVESIINLFQVVHNSYNRP
AYSPGHKTQPFLHDGYAVSWNTVEDSIIRTGFQGESGHDIKITAENTPLPIAGVLLPTI
PGKLDVNKSKTHISVNGRKIRMRCRAIDGDVTFCRPKSPVYVGNGVHANLHVAFHR
SSSEKIHSNEISSDSIGVLGYQKTVDHTKVNSKLSLFFEIKSHHHHHH

PglS₁₁₀₂₆₄

SEQ ID NO: 165
MNFLISKLKFYVLFIGIVCFCLTFILPNTSYFSSSLFKEIVVVLGFLILLTNQILSLKEIILP
KKAPLLFILFLFLFLFLFFQYLFKLIISFQDLFFNLIYISVFFLSIIFGLNSKKYNQIILIHWI
LFSLIFSALISFLIGLNQKIRIIESPYLFGVSYNGRATANLGQPNQLSTLTLMAFFSLFYL
KKYYKINKLFFYSIIISLIFCNVLTQSRSAWLSVILISIFFITKFPDKKNVLSVFCLNLVF
WLSTILIPFFFNYFYPIGNSYTTLDRMKLSSSRFDIWPQLFLATFDKPFLGYGAGQVGL
AQIESISNVSTRGEWFTYSHNIFLDFVIWYGWIVGSLVSFFIISLLIKISKSDLNRNETYL
FVIILVFFFHCLLEYPYSYFYFLIPIGIISGFLLKLKSDDIFVLKKMYLCIVVFLSWLLFTL
FTYQLIELGEKKESYSLQYLFKSSVKPIQSNLFILDGYSEKLDIEYLDYCYLIKNKDKE
FFRRVAYRYPSTVSVSKYYSTQSDNLKNAENIVQAYQVISNRVYQPHIKKCNN

PglS_{ADP1}

SEQ ID NO: 166
MNSIFKKIKNYTIVSGVFFLGSAFIIPNTSNLSSTLYKELIAVLGLLILLTVKSFDYKKILI
PKNFYWFLFVIFIIFIQLIVGEIYFFQDFFFSISFLVILFLSFLLGFNERLNGDDLIVKKIA
WIFIIVVQISFLIAINQKIEIVQNFFLFSSSYNGRSTANLGQPNQFSTLILITLFLLCYLRE
KNSLNNMVFNILSFCLIFANVMTQSRSAWISVILISLLYLLKFQKKIELRRVIFFNIVFW
TLVYCVPLLFNLIFFQKNSYSTFDRLTMGSSRFEIWPQLLKAVFHKPFIGYGWGQTGV
AQLETINKSSTKGEWFTYSHNLFLDLMLWNGFFIGLIISILILCFLIELYSSIKNKSDLFL
FFCVVAFFVHCLLEYPFAYTYFLIPVGFLCGYISTQNIKNSISYFNLSKRKLTLFLGCC
WLGYVAFWVEVLDISKKNEIYARQFLFSNHVKFYNIENYILDGFSKQLDFQYLDYCE
LKDKYQLLDFKKVAYRYPNASIVYKYYSISAEMKMDQKSANQIIRAYSVIKNQKIIKP
KLKFCSIEY

PglS_{GFJ-2}

SEQ ID NO: 167
MINILNKFKDCLIIIGLGCLCLAFFLPNTSNFSSSLFKEFFAVLGFLFILTVQFFFLKKIV
VPSKLFILFILFIFLFIQYVFNLIINFQDLFFNLIYISIFFLSIIFGLNSKKYNNSVLIHWILFS
LIFSALVSFLISLNQKIRIIESPYLFCVSYNGRATANLGQPNQLSTLTLMAFFSLFYLKK
YYKINKLFFYTIIISLIFCNVLTQSRSAWLSVILISTFFITKFPDKKNVLSVFCLNLVFWL
STILIPFFFNYFYPIGNSYTTLDRMKLSSSRFDIWPQLFLATFDKPFLGYGAGQVGLAQI
ESISNASTRGEWFTYSHNIFLDFVIWYGWIVGSLVSFFIISLLIKISKSDLNRNKTYLFIII
LVFFFHCLLEYPYSYFYFLIPIGIISGFLLKLKSDGVFVLKKIYLCIVIFLSWLLFALFTY
QLIELDEKKESYSLQYLFKSSVKPIQSNLFILDGYSEKLDIEYLDYCYLIKNRDKEFFR
RVAYRYPSTVSVSKYYSTQSDNLKNAENIVQAYQVISNRVYQPNIKKCNN

PglS₅₀ᵥ₁

SEQ ID NO: 168
MRLYLSFLLLGLSYLSPNSSLLWPNSLQDFFAILSLILLLLTFNLNNFLINKYLFLVFLL
LISIPVIQYNLKIIYFKQELFLSCLYITIFFSSIFLGSSIHNSQKVFIKFSIFFLVIGVLCVLIQ
IFQWIAVYSSIFINDLNSSRLSANIGQPNQLASLLSISLISCLILYKNKKIKVLIFSTCSVLI
IFGIVLTQSRTSWLIFILIILFSYFKKNLKLTKYVTIFSTIFYGLLITYPFFYNSIHKKDISII
QRLNSDYSRLDIWQQMLFAIIERPWFGYGWNQTSVAQTEISLYHTTSIWIEYSHNLFL
DFLIWNGIPLGIILITIIIFWFIYMYVNIKDLNSFMILIIISSFFIHCLLEFPFAYAYFIFPIGL
YIGIINKRYLKYNYFNFNNWNYIFGLIIIFLLFFIVKDYIKITEKHKEYSLKYFSDNSILP
NKLDIYLLDSLNVKEDIQYLDICYLIKIYNSEEIRNNFLRYPTNKSAVSLYYISLYNKN
VSLETISFMKWKFQNLDLNTLKINKRCNTL

PglS₄₄₆₆

SEQ ID NO: 169
MSFYCYKYLNLFGIFLMGVAYFSTITLFFSTTFYKEIFAVAGLLFFLTALCFQYKIVST
QLLLFNLALLLIPMIQYAFGIIFFLQDALLSTVYLCIFLCSILVGVNFKANHQTNILNIFL
AMLVFVGCISVLMAFNQRFMWFNSYLLFSSSYGNRATANLAQPNQLSTLLIMSLFSL
FYLYQAQKIKKIIMYGITFILLIGIVMTQSRSAWASCIVLSALYLYYYHQKQDIINVIKL
NVVFIGLTLCIPFLLNVLTYSQASTAIDRLQGGSTRFKIWPQLLHAVMEQPWTGYGW
GQVDVAQLSTMTPTSTKKELFTYSHNLFLDLLLWNGLVLGTLLSLLIIYILYRCYMNL
QYKQDLLLFLGFMAFFVHSCLEYPYAYTYFLIPAGMFLGYVSYQQNIKEVLIQINKKL
YVIFLILLCIIFGCFLIEVNHLNEKSDLYARQNLFHEKVDFNDQKFYFLDGYSTSLDFQ
TIPYCNLVQYYPLITFKQIAYRYPSALTIAKYWLFSQKQQQMVRDAEQLRQAYVLLT
KSGQHTFNNKVCN

PglS_{SFC}

SEQ ID NO: 170
MQLILIILGLSYLNPNSFLPWPNAMQDFCAMVALILLTATQFIKKNIQINKNTFYLFLFI
LSIPIIQFLFNILFFKQELFLSILYISIFFLSIIYGINQKEASNRIIKVSFFFVSVGIVCVFIQII
QWTNIYYSPFILESNYLRPSANLGQPNNLATLLFICLFSNLYIFKNKKINTSFYISINIFII
FGIALTQSRTSWIVFIALLILSHFKKELKLFKTIMINSILFFILVLITPYITLFYHGKGLTII
ERINSDYSRLSIWKQIIIAITNKPLTGYGWNQTSVAQTQISLKYPIKVWLEYSHNMFLD
ILVWTGIPIGLLIITLINKWLFKTYQNIKNTNQLIIFFIIISFFIHCMFEFPFAYAYFLIPVGI
YIGFLNKQDYNIITINIFTILLFLLISTLLTIITIDYMVLSEKRNNYSTKYLFSKKISPLESN
IKILDALDLHNDILFLNDCYILKNKSIKNIKHIFYRYPTNKNIVIYYRFSLYYKNSSKEV
IEYMKLKYPNFDSNQSKYNMCN

Pgl_{SP5312}

SEQ ID NO: 171
MPIFYFILGLSYLSPIFMQPWVSAFQDLCAIIAIILLMSIQSYRKNIEIDRRVLYVFGFIV
CIPLVQYLFGILFFTQELVLSLIYISVFFLSIISGANFNRSYKNEEKLSFFFVFIGLSCVFIQ
LIQWSGLYHSALILDSSSRRPFANIGQPNNLATLLFIGFFSNILLFKNNRLKAKFYFLIS
AVLMTGIVLTQSRTSWLVFVSVLLLAFFKSKLELFSIMLKSSVLFFCLVLILPYITLFFH
DQGLTVTERISSDSSRLYIWKQMLIAIMDKPWFGYGWNQTSVAQTSVTLKYPLDIWL
EYSHNLFLDLIVWTGIPIGLSIIGIIIIWFLQTFKKINTLNQLLYFFIIAAFLIHCMLEYPFA
YAYFLVPIGLYVGMLHQQLYETKNLKFKSLVITLVSILIITIIIISRDYFVLSDKRTIYTS
ESLFSEQVKPAFSKVLVLDALDVNNDILFLNRCYVLKKNTIENFKSNFYRYPTRMNL
VMYYKSTIYYEKNSRDAERYMTAWYPDYKQNLSQYDICS

ComP_{P5312}

SEQ ID NO: 7
MNAQKGFTLIELMIVIAIIGILAAIALPAYTDYTTRARVSEALTTASAM
KATVSENIISKGGTSIDEDSACIGVATVGSDASAATKNVQKSVCDKGVI
TVTTTPDAKSVPLILTPSYSGDGVEWTCTTTADKKYVPAECR

ComP Δ28_{P5312}

SEQ ID NO: 15
MNAQKGFTLIELMIVIAIIGILAAIALPAYTDYTTRARVSEALTTASAM
KATVSENIISKGGTSIDEDSACIGVATVGSDASAATKNVQKSVCDKGVI
TVTTTPDAKSVPLILTPSYSGDGVEWTCTTTADKKYVPAECR

PglS_{ANT_H59}

SEQ ID NO: 172
MLIFYIMLGLSYLSPNIFLPWLNALQDLFAIFALIILVSKQSYRKDIEIDERVIYVFGLIA
LIPLVQYLFGLLFFTQELVLSLIYISAFFLSIISGINLTKSFKEIEKISFSFIFISLSCVLLQLI
QWSNIYHSALLLDSSSRRPFANIGQPNNLATLLFIGFFSNILLFKNNKIKIYLYLLVSAT
LMTGIVLTQSRTSWLVFIAVLFITFLKKKLNLFSTMLKSSIAFLFLVLTLPYITLFFHDQ
GLTVIERISSDSSRLYIWKQMLIAIIDKPWFGYGWNQTSVAQTSVTLKYPLNIWLEYS
HNLFLDIIVWTGIPIGISIITIIIIWFLQTFKKINTPNQLIYFLIITAFFIHCMLEFPFAYAYF
LLPVGLYVGILHQQVYETKNSKVKGLVMTIVTVLIVAVIIISRDYFLFNNKRTIYASKN
LFSQQIQPISSKILLLNALDINNDILFLDECYVLKNNKFKVLRNSFYRYPTNKNLITYY
KSAIYNNQNTQYPEKYMQKEYSNFKSSPAIYNNCSKL

ComP_{ANT_H59}

SEQ ID NO: 8
MNTAQKGFTLIELMIVIAIIGILAAIAIPAYSDYTARARVTEAVTTASS
MKATVSENIISKGGTTIGAGSCAGVSLIGASNKTKNVLSSTCTDTTGVI
LVTTTADAKSVPLTLTPTYTGDAVTWKCTTTSDFTKYVPAECRPH

ComPΔ29_{ANT_H59}

SEQ ID NO: 16
MNTAQKGFTLIELMIVIAIIGILAAIAIPAYSDYTARARVTEAVTTASSMKATVSENIIS
KGGTTIGAGSCAGVSLIGASNKTKNVLSSTCTDTTGVILVTTTADAKSVPLTLTPTYT
GDAVTWKCTTTSDFTKYVPAECRPH

| | | |
|---|---|---|
| iGTcc | CTGVTQIASGASAATTNVASAQC | (SEQ ID NO: 30); |
| iGTss | STGVTQIASGASAATTNVASAQS | (SEQ ID NO: 31); |
| iGTccΔ0-1 | CTGVTQIASGASAATINVASAQ | (SEQ ID NO: 32); |
| iGTccΔ0-2 | CTGVTQIASGASAATINVASA | (SEQ ID NO: 33); |
| iGTccΔ0-3 | CTGVTQIASGASAATTNVAS | (SEQ ID NO: 34); |
| iGTccΔ0-4 | CTGVTQIASGASAATTNVA | (SEQ ID NO: 35); |
| iGTccΔ0-5 | CTGVTQIASGASAATTNV | (SEQ ID NO: 36); |
| iGTccΔ0-6 | CTGVTQIASGASAATTN | (SEQ ID NO: 37); |
| iGTccΔ0-7 | CTGVTQIASGASAATT | (SEQ ID NO: 38); |
| iGTccΔ0-8 | CTGVTQIASGASAAT | (SEQ ID NO: 39); |
| iGTccΔ0-9 | CTGVTQIASGASAA | (SEQ ID NO: 40); |
| iGTccΔ0-10 | CTGVTQIASGASA | (SEQ ID NO: 41); |
| iGTccΔ0-11 | CTGVTQIASGAS | (SEQ ID NO: 42); |
| iGTccΔ1-0 | TGVTQIASGASAATTNVASAQC | (SEQ ID NO: 43); |
| iGTccΔ1-1 | TGVTQIASGASAATTNVASAQ | (SEQ ID NO: 44); |
| iGTccΔ1-2 | TGVTQIASGASAATTNVASA | (SEQ ID NO: 45); |
| iGTccΔ1-3 | TGVTQIASGASAATTNVAS | (SEQ ID NO: 46); |
| iGTccΔ1-4 | TGVTQIASGASAATTNVA | (SEQ ID NO: 47); |
| iGTccΔ1-5 | TGVTQIASGASAATTNV | (SEQ ID NO: 48); |
| iGTccΔ1-6 | TGVTQIASGASAATTN | (SEQ ID NO: 49); |
| iGTccΔ1-7 | TGVTQIASGASAATT | (SEQ ID NO: 50); |
| iGTccΔ1-8 | TGVTQIASGASAAT | (SEQ ID NO: 51); |
| iGTccΔ1-9 | TGVTQIASGASAA | (SEQ ID NO: 52); |
| iGTccΔ1-10 | TGVTQIASGASA | (SEQ ID NO: 53); |
| iGTccΔ1-11 | TGVTQIASGAS | (SEQ ID NO: 54); |
| iGTccΔ2-0 | GVTQIASGASAATINVASAQC | (SEQ ID NO: 55); |
| iGTccΔ2-1 | GVTQIASGASAATTNVASAQ | (SEQ ID NO: 56); |
| iGTccΔ2-2 | GVTQIASGASAATTNVASA | (SEQ ID NO: 57); |
| iGTccΔ2-3 | GVTQIASGASAATTNVAS | (SEQ ID NO: 58); |
| iGTccΔ2-4 | GVTQIASGASAATTNVA | (SEQ ID NO: 59); |
| iGTccΔ2-5 | GVTQIASGASAATTNV | (SEQ ID NO: 60); |
| iGTccΔ2-6 | GVTQIASGASAATTN | (SEQ ID NO: 61); |
| iGTccΔ2-7 | GVTQIASGASAATT | (SEQ ID NO: 61); |
| iGTccΔ2-8 | GVTQIASGASAAT | (SEQ ID NO: 63); |
| iGTccΔ2-9 | GVTQIASGASAA | (SEQ ID NO: 64); |
| iGTccΔ2-10 | GVTQIASGASA | (SEQ ID NO: 65); |
| iGTccΔ2-11 | GVTQIASGAS | (SEQ ID NO: 66); |
| iGTccΔ3-0 | VTQIASGASAATTNVASAQC | (SEQ ID NO: 67); |
| iGTccΔ3-1 | VTQIASGASAATINVASAQ | (SEQ ID NO: 68); |
| iGTccΔ3-2 | VTQIASGASAATTNVASA | (SEQ ID NO: 69); |
| iGTccΔ3-3 | VTQIASGASAATTNVAS | (SEQ ID NO: 70); |
| iGTccΔ3-4 | VTQIASGASAATTNVA | (SEQ ID NO: 71); |
| iGTccΔ3-5 | VTQIASGASAATTNV | (SEQ ID NO: 72); |
| iGTccΔ3-6 | VTQIASGASAATTN | (SEQ ID NO: 73); |
| iGTccΔ3-7 | VTQIASGASAATT | (SEQ ID NO: 74); |
| iGTccΔ3-8 | VTQIASGASAAT | (SEQ ID NO: 75); |
| iGTccΔ3-9 | VTQIASGASAA | (SEQ ID NO: 76); |
| iGTccΔ3-10 | VTQIASGASA | (SEQ ID NO: 77); |
| iGTccΔ3-11 | VTQIASGASS | (SEQ ID NO: 78); |
| iGTccΔ4-0 | TQIASGASAATTNVASAQC | (SEQ ID NO: 79); |
| iGTccΔ4-1 | TQIASGASAATTNVASAQ | (SEQ ID NO: 80); |
| iGTccΔ4-2 | TQIASGASAATTNVASA | (SEQ ID NO: 81); |
| iGTccΔ4-3 | TQIASGASAATTNVAS | (SEQ ID NO: 82); |
| iGTccΔ4-4 | TQIASGASAATTNVA | (SEQ ID NO: 83); |
| iGTccΔ4-5 | TQIASGASAATTNV | (SEQ ID NO: 84); |
| iGTccΔ4-6 | TQIASGASAATTN | (SEQ ID NO: 85); |
| iGTccΔ4-7 | TQIASGASAATT | (SEQ ID NO: 86); |
| iGTccΔ4-8 | TQIASGASAAT | (SEQ ID NO: 87); |
| iGTccΔ4-9 | TQIASGASAA | (SEQ ID NO: 88); |
| iGTccΔ4-10 | TQIASGASA | (SEQ ID NO: 89); |
| iGTccΔ4-11 | TQIASGAS | (SEQ ID NO: 90); |
| iGTccΔ5-0 | QIASGASAATTNVASAQC | (SEQ ID NO: 91); |
| iGTccΔ5-1 | QIASGASAATTNVASAQ | (SEQ ID NO: 92); |
| iGTccΔ5-2 | QIASGASAATTNVASA | (SEQ ID NO: 93); |
| iGTccΔ5-3 | QIASGASAATTNVAS | (SEQ ID NO: 94); |
| iGTccΔ5-4 | QIASGASAATTNVA | (SEQ ID NO: 95); |
| iGTccΔ5-5 | QIASGASAATTNV | (SEQ ID NO: 96); |
| iGTccΔ5-6 | QIASGASAATTN | (SEQ ID NO: 97); |
| iGTccΔ5-7 | QIASGASAATT | (SEQ ID NO: 98); |
| iGTccΔ5-8 | QIASGASAAT | (SEQ ID NO: 99); |
| iGTccΔ5-9 | QIASGASAA | (SEQ ID NO: 100); |
| iGTccΔ5-10 | QIASGASA | (SEQ ID NO: 101); |
| iGTccΔ5-11 | QIASGASA | (SEQ ID NO: 102); |
| iGTccΔ6-0 | IASGASAATTNVASAQC | (SEQ ID NO: 103); |
| iGTccΔ6-1 | IASGASAATTNVASAQ | (SEQ ID NO: 104); |
| iGTccΔ6-2 | IASGASAATTNVASA | (SEQ ID NO: 105); |
| iGTccΔ6-3 | IASGASAATTNVAS | (SEQ ID NO: 106); |
| iGTccΔ6-4 | IASGASAATTNVA | (SEQ ID NO: 107); |
| iGTccΔ6-5 | IASGASAATTNV | (SEQ ID NO: 108); |
| iGTccΔ6-6 | IASGASAATTN | (SEQ ID NO: 109); |
| iGTccΔ6-7 | IASGASAATT | (SEQ ID NO: 110); |
| iGTccΔ6-8 | IASGASAAT | (SEQ ID NO: 111); |
| iGTccΔ6-0 | IASGASAA | (SEQ ID NO: 112); |
| iGTccΔ6-10 | IASGASA | (SEQ ID NO: 113); |
| iGTccΔ6-11 | IASGAS | (SEQ ID NO: 114); |
| iGTccΔ7-0 | ASGASAATINVASAQC | (SEQ ID NO: 115); |
| iGTccΔ7-1 | ASGASAATTNVASAQ | (SEQ ID NO: 116); |
| iGTccΔ7-2 | ASGASAATINVASA | (SEQ ID NO: 117); |
| iGTccΔ7-3 | ASGASAATTNVAS | (SEQ ID NO: 118); |
| iGTccΔ7-4 | ASGASAATTNVA | (SEQ ID NO: 119); |
| iGTccΔ7-5 | ASGASAATTNV | (SEQ ID NO: 120); |
| iGTccΔ7-6 | ASGASAATTN | (SEQ ID NO: 121); |
| iGTccΔ7-7 | ASGASAATT | (SEQ ID NO: 122); |
| iGTccΔ7-8 | ASGASAAT | (SEQ ID NO: 123); |
| iGTccΔ7-9 | ASGASAA | (SEQ ID NO: 124); |
| iGTccΔ7-10 | ASGASA | (SEQ ID NO: 125); |
| iGTccΔ7-11 | ASGAS | (SEQ ID NO: 126); |
| iGTccΔ8-0 | SGASAATTNVASAQC | (SEQ ID NO: 127); |
| iGTccΔ8-1 | SGASAATTNVASAQ | (SEQ ID NO: 128); |
| iGTccΔ8-2 | SGASAATTNVASA | (SEQ ID NO: 129); |
| iGTccΔ8-3 | SGASAATINVAS | (SEQ ID NO: 130); |
| iGTccΔ8-4 | SGASAATTNVA | (SEQ ID NO: 131); |
| iGTccΔ8-5 | SGASAATTNV | (SEQ ID NO: 132); |
| iGTccΔ8-6 | SGASAATTN | (SEQ ID NO: 133); |
| iGTccΔ8-7 | SGASAATT | (SEQ ID NO: 134); |
| iGTccΔ8-8 | SGASAAT | (SEQ ID NO: 135); |
| iGTccΔ8-9 | SGASAA | (SEQ ID NO: 136); |
| iGTccΔ8-10 | SGASA | (SEQ ID NO: 137); |
| iGTccΔ9-0 | GASAATINVASAQC | (SEQ ID NO: 138); |
| iGTccΔ9-1 | GASAATTNVASAQ | (SEQ ID NO: 139); |
| iGTccΔ9-2 | GASAATTNVASA | (SEQ ID NO: 140); |
| iGTccΔ9-3 | GASAATTNVAS | (SEQ ID NO: 141); |
| iGTccΔ9-4 | GASAATTNVA | (SEQ ID NO: 142); |
| iGTccΔ9-5 | GASAATTNV | (SEQ ID NO: 143); |
| iGTccΔ9-6 | GASAATTN | (SEQ ID NO: 144); |
| iGTccΔ9-7 | GASAATT | (SEQ ID NO: 145); |
| iGTccΔ9-8 | GASAAT | (SEQ ID NO: 146); |
| iGTccΔ9-9 | GASAA | (SEQ ID NO: 147); |
| iGTccΔ10-0 | ASAATTNVASAQC | (SEQ ID NO: 148); |
| iGTccΔ10-1 | ASAATTNVASAQ | (SEQ ID NO: 149); |
| iGTccΔ10-2 | ASAATTNVASA | (SEQ ID NO: 150); |
| iGTccΔ10-3 | ASAATTNVAS | (SEQ ID NO: 151); |
| iGTccΔ10-4 | ASAATTNVA | (SEQ ID NO: 152); |
| iGTccΔ10-5 | ASAATTNV | (SEQ ID NO: 153); |
| iGTccΔ10-6 | ASAATTN | (SEQ ID NO: 154); |
| iGTccΔ10-7 | ASAATT | (SEQ ID NO: 155); |
| iGTccΔ10-8 | ASAAT | (SEQ ID NO: 156); |
| iGTccΔ11-0 | SAATTNVASAQC | (SEQ ID NO: 157); |
| iGTccΔ11-1 | SAATTNVASAQ | (SEQ ID NO: 158); |
| iGTccΔ11-2 | SAATTNVASA | (SEQ ID NO: 159); |
| iGTccΔ11-3 | SAATTNVAS | (SEQ ID NO: 160); |
| iGTccΔ11-4 | SAATTNVA | (SEQ ID NO: 161); |
| iGTccΔ11-5 | SAATTNV | (SEQ ID NO: 161); |
| iGTccΔ11-6 | SAATTN | (SEQ ID NO: 163); |
| iGTccΔ11-7 | SAATT | (SEQ ID NO: 164). |

### REFERENCES

1. Szymanski, C. M., Yao, R., Ewing, C. P., Trust, T. J. & Guerry, P. Evidence for a system of general protein glycosylation in Campylobacter jejuni. Mol Microbiol 32, 1022-1030, doi:10.1046/j.1365-2958.1999.01415.x (1999).
2. Wacker, M. et al. N-linked glycosylation in Campylobacter jejuni and its functional transfer into E. coli. Science 298, 1790-1793, doi:10.1126/science.298.5599.1790 (2002).
3. Nothaft, H. & Szymanski, C. M. Protein glycosylation in bacteria: sweeter than ever. Nat Rev Microbiol 8, 765-778, doi:10.1038/nrmicro2383 (2010).
4. Schaffer, C. & Messner, P. Emerging facets of prokaryotic glycosylation. FEMS Microbiol Rev 41, 49-91, doi:10.1093/femsre/fuw036 (2017).
5. Harding, C. M. & Feldman, M. F. Glycoengineering bioconjugate vaccines, therapeutics, and diagnostics in E. coli. Glycobiology 29, 519-529, doi:10.1093/glycob/cwz031 (2019).
6. Valvano, M. A. Export of O-specific lipopolysaccharide. Front Biosci 8, s452-471, doi:10.2741/1079 (2003).
7. Hug, I. & Feldman, M. F. Analogies and homologies in lipopolysaccharide and glycoprotein biosynthesis in bacteria. Glycobiology 21, 138-151, doi:10.1093/glycob/cwql48 (2011).
8. Raetz, C. R. & Whitfield, C. Lipopolysaccharide endotoxins. Annu Rev Biochem 71, 635-700, doi:10.1146/annurev.biochem.71.110601.135414 (2002).
9. Wacker, M. et al. Substrate specificity of bacterial oligosaccharyltransferase suggests a common transfer mechanism for the bacterial and eukaryotic systems. Proc Natl Acad Sci USA 103, 7088-7093, doi:10.1073/pnas.0509207103 (2006).
10. Faridmoayer, A. et al. Extreme substrate promiscuity of the Neisseria oligosaccharyl transferase involved in protein O-glycosylation. J Biol Chem 283, 34596-34604, doi:10.1074/jbc.M807113200 (2008).
11. Feldman, M. F. et al. Engineering N-linked protein glycosylation with diverse O antigen lipopolysaccharide structures in Escherichia coli. Proc Natl Acad Sci USA 102, 3016-3021, doi:10.1073/pnas.0500044102 (2005).
12. Kowarik, M. et al. Definition of the bacterial N-glycosylation site consensus sequence. EMBO J 25, 1957-1966, doi:10.1038/sj.emboj.7601087 (2006). 13. Faridmoayer, A., Fentabil, M. A., Mills, D. C., Klassen, J. S. & Feldman, M. F.
Functional characterization of bacterial oligosaccharyltransferases involved in O-linked protein glycosylation. J Bacteriol 189, 8088-8098, doi:10.1128/JB.01318-07 (2007).
14. Vik, A. et al. Broad spectrum O-linked protein glycosylation in the human pathogen Neisseria gonorrhoeae. Proc Natl Acad Sci USA 106, 4447-4452, doi:10.1073/pnas.0809504106 (2009).
15. Pan, C. et al. Biosynthesis of Conjugate Vaccines Using an O-Linked Glycosylation System. MBio 7, e00443-00416, doi:10.1128/mBio.00443-16 (2016).
16. Harding, C. M. et al. Acinetobacter strains carry two functional oligosaccharyltransferases, one devoted exclusively to type IV pilin, and the other one dedicated to O-glycosylation of multiple proteins. Mol Microbiol 96, 1023-1041, doi:10.1111/mmi.12986 (2015).
17. Harding, C. M. et al. A platform for glycoengineering a polyvalent pneumococcal bioconjugate vaccine using E. coli as a host. Nat Commun 10, 891, doi:10.1038/s41467-019-08869-9 (2019).
18. Wacker, M. et al. Prevention of Staphylococcus aureus infections by glycoprotein vaccines synthesized in Escherichia coli. J Infect Dis 209, 1551-1561, doi:10.1093/infdis/jit800 (2014).
19. Hatz, C. F. et al. Safety and immunogenicity of a candidate bioconjugate vaccine against Shigella dysenteriae type 1 administered to healthy adults: A single blind, partially randomized Phase I study. Vaccine 33, 4594-4601, doi:10.1016/j.vaccine.2015.06.102 (2015).
20. Huttner, A. et al. Safety, immunogenicity, and preliminary clinical efficacy of a vaccine against extraintestinal pathogenic Escherichia coli in women with a history of recurrent urinary tract infection: a randomised, single-blind, placebo-controlled phase 1b trial. Lancet Infect Dis 17, 528-537, doi:10.1016/51473-3099(17)30108-1 (2017).
21. Sun, P. et al. Design and production of conjugate vaccines against S. Paratyphi A using an O-linked glycosylation system in vivo. NPJ Vaccines 3, 4, doi:10.1038/s41541-017-0037-1 (2018).
22. van den Dobbelsteen, G. et al. Immunogenicity and safety of a tetravalent E. coli O-antigen bioconjugate vaccine in animal models. Vaccine 34, 4152-4160, doi:10.1016/j.vaccine.2016.06.067 (2016).
23. Geno, K. A. et al. Pneumococcal Capsules and Their Types: Past, Present, and Future. Clin Microbiol Rev 28, 871-899, doi:10.1128/CMR.00024-15 (2015).
24. Pan, Y. J. et al. Genetic analysis of capsular polysaccharide synthesis gene clusters in 79 capsular types of Klebsiella spp. Sci Rep 5, 15573, doi:10.1038/srep15573 (2015).
25. Berti, F. et al. Structure of the type IX group B Streptococcus capsular polysaccharide and its evolutionary relationship with types V and VII. J Biol Chem 289, 23437-23448, doi:10.1074/jbc.M114.567974 (2014).
26. Feldman, M. F. et al. A promising bioconjugate vaccine against hypervirulent Klebsiella pneumoniae. Proc Natl Acad Sci USA 116, 18655-18663, doi:10.1073/pnas.1907833116 (2019).
27. Ihssen, J. et al. Production of glycoprotein vaccines in Escherichia coli. Microb Cell Fact 9, 61, doi:10.1186/1475-2859-9-61 (2010).
28. Gerber, S. et al. Mechanism of bacterial oligosaccharyltransferase: in vitro quantification of sequon binding and catalysis. J Biol Chem 288, 8849-8861, doi:10.1074/jbc.M112.445940 (2013).
29. Kay, E. J., Yates, L. E., Terra, V. S., Cuccui, J. & Wren, B. W. Recombinant expression of Streptococcus pneumoniae capsular polysaccharides in Escherichia coli. Open Biol 6, 150243, doi:10.1098/rsob.150243 (2016).
30. Garcia-Quintanilla, F., Iwashkiw, J. A., Price, N. L., Stratilo, C. & Feldman, M. F. Production of a recombinant vaccine candidate against Burkholderia pseudomallei exploiting the bacterial N-glycosylation machinery. Front Microbiol 5, 381, doi:10.3389/fmicb.2014.00381 (2014).
31. Berti, F. & Adamo, R. Antimicrobial glycoconjugate vaccines: an overview of classic and modem approaches for protein modification. Chem Soc Rev 47, 9015-9025, doi:10.1039/c8cs00495a (2018).
32. Goffin, P., Dewerchin, M., De Rop, P., Blais, N. & Dehottay, P. High-yield production of recombinant CRM197, a non-toxic mutant of diphtheria toxin, in the periplasm of Escherichia coli. Biotechnol J 12, doi: 10.1002/biot.201700168 (2017).
33. Wedekind, J. E. et al. Refined crystallographic structure of Pseudomonas aeruginosa exotoxin A and its implications for the molecular mechanism of toxicity. J Mol Biol 314, 823-837, doi: 10.1006/jmbi.2001.5195 (2001).

What is claimed is:

1. A glycoconjugate comprising an oligo- or polysaccharide covalently linked to a fusion protein:
wherein the fusion protein comprises a ComP protein (ComP) glycosylation fragment;
wherein the ComP glycosylation fragment does not contain a cysteine residue corresponding to the conserved cysteine residue at position 71 of ComP₁₁₀₂₆₄ (SEQ ID NO: 1) and/or does not contain a cysteine residue corresponding to the conserved cysteine residue at position 93 of ComP₁₁₀₂₆₄ (SEQ ID NO: 1);
wherein the ComP glycosylation fragment is located internally within the fusion protein; and
wherein the fusion protein is glycosylated with the oligo- or polysaccharide on the ComP glycosylation fragment at serine residue corresponding to the conserved serine residue at position 82 of ComP₁₁₀₂₆₄ (SEQ ID NO: 1);
optionally, wherein the glycoconjugate is immunogenic;
optionally, wherein the ComP glycosylation fragment is solvent (or surface)-exposed;
optionally, wherein the ComP glycosylation fragment is integrated into a C₁₀ β-turn, β-turn, β-twist, β-loop, U turn, reverse turn, chain reversal, or a hairpin loop of the fusion protein.

2. The glycoconjugate of claim 1, wherein the ComP glycosylation fragment has a length of from 5 to 22 amino acids in length, has a length of from 10 to 22 amino acids in length, has a length of from 11 to 22 amino acids in length, has a length of from 5 to 21 amino acids in length, has a length of from 10 to 21 amino acids in length, or has a length of from 11 to 21 amino acids in length;
optionally, wherein the fragment has at least 1, 2, 3, 4, or 5 amino acid residues N-terminal to the serine residue corresponding to the conserved serine residue at position 82 of SEQ ID NO: 1 and/or wherein the fragment has at least 1, 2, 3, 4, or 5 amino acid residues C-terminal to the serine residue corresponding to the conserved serine residue at position 82 of SEQ ID NO: 1.

3. The glycoconjugate of claim 1, wherein the amino acid sequence of the ComP glycosylation fragment does not extend in the N-terminus direction beyond the amino acid residue corresponding to position 72 of ComP₁₁₀₂₆₄ (SEQ ID NO: 1) and/or does not extend in the C-terminus beyond the amino acid residue corresponding to position 92 of ComP₁₁₀₂₆₄ (SEQ ID NO: 1).

4. The glycoconjugate of claim 1, wherein the ComP protein comprises an amino acid sequence that is at least 50%, 60%, 70%, 75%, 80%, 85%, 90%, 95%, 96%, 97%, 98%, 99%, or 100% identical to SEQ ID NO: 9 (ComPΔ28₁₁₀₂₆₄) SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59});
optionally, wherein the ComP protein comprises SEQ ID NO: 9 (ComPΔ28₁₁₀₂₆₄), SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59}).

5. The glycoconjugate of claim 1, wherein the ComP glycosylation fragment comprises or consists of the amino acid consensus sequence of:
(SEQ ID NO: 17)
X₁GVX₄X₅X₆X₇X₈X₉ASX₁₂X₁₃TX₁₅NVX₁₈X₁₉X₂₀X₂₁
wherein: X₁ is V, T, A, or I;
X₄ is Q, T, E, A, or S;
X₅ is E, Q, T, or L;
X₆ is I or V;
X₇ is S, N, A, or G;
X₈ is S or no amino acid;
X₉ is G, D, or no amino acid;
X₁₂ is N, S, or A;
X₁₃ is A, S, or K;
X₁₅ is T, S, or K;
X₁₈ is A, E, Q, or L;
X₁₉ is T, S, or K;
X₂₀ is A or S; and
X₂₁ is T, Q, A, or V;
or a fragment of thereof of at least 5, 6, 7, 8, 9, 10, or 11 amino acids in length comprising the serine (S) residue at position 11 of SEQ ID NO: 17,
optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17;
or a variant of the amino acid consensus sequence of SEQ ID NO: 17 or the fragment thereof, having one, two, or three amino acid substitutions, additions, and/or deletions, wherein the variant maintains the serine (S) residue at position 11 of SEQ ID NO: 17,
optionally, wherein the variant has at least 1, 2, 3, 4, 5, or 6 amino acid residues N-terminal to the serine (S) residue at position 11 of SEQ ID NO: 17 and/or wherein the variant has at least 1, 2, 3, 4, 5, or 6 amino acid residues C-terminal to the serine (S) residue at position 11 of SEQ ID NO: 17;
optionally, wherein the ComP glycosylation fragment can be glycosylated when located internally in a fusion protein; and
optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

6. The glycoconjugate of claim 1, wherein the ComP glycosylation fragment comprises or consists of the amino acid consensus sequence of:
(SEQ ID NO: 17)
X₁GVX₄X₅X₆X₇X₈X₉ASX₁₂X₁₃TX₁₅NVX₁₈X₁₉X₂₀X₂₁
wherein: X₁ is V, T, A, or I;
X₄ is Q, T, E, A, or S;
X₅ is E, Q, T, or L;
X₆ is I or V;
X₇ is S, N, A, or G;
X₈ is S or no amino acid;
X₉ is G, D, or no amino acid;
X₁₂ is N, S, or A;
X₁₃ is A, S, or K;
X₁₅ is T, S, or K;
X₁₈ is A, E, Q, or L;
X₁₉ is T, S, or K;
X₂₀ is A or S; and
X₂₁ is T, Q, A, or V;
or a fragment of thereof of at least 5, 6, 7, 8, 9, 10, or 11 amino acids in length comprising the serine (S) residue at position 11 of SEQ ID NO: 17,
optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17;
optionally, wherein the ComP glycosylation fragment can be glycosylated when located internally in a fusion protein; and
optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

7. The glycoconjugate of claim 1, wherein the fusion protein comprises a carrier protein selected from the group consisting of Pseudomonas aeruginosa Exotoxin A (EPA), CRM₁₉₇, cholera toxin B subunit, tetanus toxin C fragment, Haemophilus influenzae Protein D, and a fragment or fragments thereof;
optionally, wherein the Pseudomonas aeruginosa Exotoxin A (EPA) carrier protein comprises the amino acid sequence of SEQ ID NO: 18, or a fragment or fragments thereof;
optionally, wherein the CRM₁₉₇ carrier protein comprises the amino acid sequence of SEQ ID NO: 24, or a fragment or fragments thereof.

8. The glycoconjugate of claim 7, wherein:
(i) the ComP glycosylation fragment is inserted between Ala489 and Arg490 relative to the PDB entity 1IKQ of Pseudomonas aeruginosa Exotoxin A (EPA) (SEQ ID NO: 19);
(ii) the ComP glycosylation fragment is inserted between Glu548 and Gly549 relative to the PDB entity 1IKQ of Pseudomonas aeruginosa Exotoxin A (EPA) (SEQ ID NO:20);
(iii) the ComP glycosylation fragment is inserted between Ala122 and Gly123 relative to the PDB entity 1IKQ of Pseudomonas aeruginosa Exotoxin A (EPA) (SEQ ID NO: 21);
(iv) the ComP glycosylation fragment is inserted between Thr355 and Gly356 relative to the PDB entity 1IKQ of Pseudomonas aeruginosa Exotoxin A (EPA) (SEQ ID NO: 22); or
(v) the ComP glycosylation fragment is inserted between Lys20 and Asp21 relative to the PDB entity 1IKQ of Pseudomonas aeruginosa Exotoxin A (EPA) (SEQ ID NO: 23).

9. The glycoconjugate of claim 7, wherein:
(i) the ComP glycosylation fragment is inserted between Asn481 and Gly482 relative to the PDB entity 4AE0 of CRM₁₉₇ (SEQ ID NO: 25);
(ii) the ComP glycosylation fragment is inserted between Asp392 and Gly393 relative to the PDB entity 4AE0 of CRM₁₉₇ (SEQ ID NO: 26);
(iii) the ComP glycosylation fragment is inserted between Glu142 and Gly143 relative to the PDB entity 4AE0 of CRM₁₉₇ (SEQ ID NO: 27);
(iv) the ComP glycosylation fragment is inserted between Asp129 and Gly130 relative to the PDB entity 4AE0 of CRM₁₉₇ (SEQ ID NO: 28); or
(v) the ComP glycosylation fragment is inserted between Asn69 and Glu70 relative to the PDB entity 4AE0 of CRM₁₉₇ (SEQ ID NO: 29).

10. The glycoconjugate of claim 1, wherein the fusion protein comprises two or more, three or more, four or more, five or more, six or more, eight or more, ten or more, fifteen or more, or twenty or more ComP glycosylation fragments;
optionally, wherein the fusion protein does not comprise more than three, more than five, more than ten, more than fifteen, more than twenty, or more than twenty five ComP glycosylation fragments.

11. The glycoconjugate of claim 1, wherein the ComP glycosylation fragments are identical.

12. The glycoconjugate of claim 1, wherein the ComP glycosylation fragments differ from each other;
optionally, wherein at least three, at least four, or at least five of the ComP glycosylation fragments all differ from each other;
optionally, wherein none of the ComP glycosylation fragments are the same.

13. The glycoconjugate of claim 1, wherein the oligo- or polysaccharide is derived from a saccharide produced by bacteria from the genus Streptococcus;
optionally, wherein the saccharide is a S. pneumoniae, S. agalactiae, or S. suis capsular polysaccharide;
optionally, wherein the saccharide is the serotype 8 capsular polysaccharide from S. pneumoniae;
optionally, wherein the saccharide is the type Ia, Ib, II, III, IV, V, VI, VII, VIII, or X capsular polysaccharide from S. agalactiae.

14. The glycoconjugate of claim 1, wherein the oligo- or polysaccharide is derived from a saccharide produced by the bacteria from the genus Klebsiella;
optionally, wherein the saccharide is a K. pneumoniae, K. varricola, K. michinganenis, or K. oxytoca capsular polysaccharide;
optionally, wherein the saccharide is a K. pneumoniae, K. varricola, K. michinganenis, or K. oxytoca O-antigen polysaccharide.

15. The glycoconjugate of claim 1, wherein oligo- or polysaccharide comprises glucose at its reducing end.

16. The glycoconjugate of claim 1, wherein the glycoconjugate is produced in vivo;
optionally, in a bacterial cell;
optionally, in Escherichia coli;
optionally, in a bacterium from the genus Klebsiella;
optionally, wherein the bacterial species is K. pneumoniae, K. varricola, K. michinganenis, or K. oxytoca.

17. The glycoconjugate of claim 1, wherein the ComP glycosylation fragment comprises or consists of an amino acid sequence of SEQ ID NO: 32-163, or 164,
or a variant thereof having one, two, or three amino acid substitutions, additions, and/or deletions,
wherein the variant comprises the serine residue corresponding to the conserved serine residue at position 82 of SEQ ID NO: 1;
optionally, wherein the ComP glycosylation fragment can be glycosylated when located internally in a fusion protein; and
optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

18. The glycoconjugate of claim 17, wherein the ComP glycosylation fragment comprises or consists of an amino acid sequence of:
iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATINVASA;
iGTccΔ2-1
(SEQ ID NO: 56)
GVTQIASGASAATTNVASAQ;
iGTccΔ2-2
(SEQ ID NO: 57)
GVTQIASGASAATINVASA;
iGTccΔ2-3
(SEQ ID NO: 58)
GVTQIASGASAATINVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN,
or the variant thereof.

19. The glycoconjugate of claim 17, wherein the ComP glycosylation fragment comprises or consists of an amino acid sequence of SEQ ID NO: 32-163, or 164,
optionally, wherein the ComP glycosylation fragment can be glycosylated when located internally in a fusion protein; and
optionally, wherein the ComP glycosylation fragment is not glycosylated when located at the N-terminal and/or C-terminal end of a fusion protein or is glycosylated at least 50% less, 60% less, 70% less, 80% less, 90% less, 95% less, or 99% less when located at the N-terminal and/or C-terminal end of a fusion protein in comparison to when it is located internally in the fusion protein.

20. The glycoconjugate of claim 19, wherein the ComP glycosylation fragment comprises or consists of an amino acid sequence of:
iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATINVASA;
iGTccΔ2-1
(SEQ ID NO: 56)
GVTQIASGASAATTNVASAQ;
iGTccΔ2-2
(SEQ ID NO: 57)
GVTQIASGASAATINVASA;
iGTccΔ2-3
(SEQ ID NO: 58)
GVTQIASGASAATINVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN,

21. The glycoconjugate of claim 1, wherein the bioconjugate is a conjugate vaccine;
optionally, wherein the conjugate vaccine is a vaccine against Streptococcus pneumoniae serotype 8.

22. The glycoconjugate of claim 21, wherein when the conjugate vaccine induces an immune response when administered to a subject.

23. The glycoconjugate of claim 22, wherein the immune response elicits long term memory (memory B and T cells), is an antibody response, and is optionally a serotype-specific antibody response.

24. The glycoconjugate of claim 23, wherein the antibody response is an IgG or IgM response.

25. The glycoconjugate of claim 24, wherein the antibody response is an IgG response; optionally an IgG1 response.

26. The glycoconjugate of claim 21, wherein the conjugate vaccine generates immunological memory in a subject administered the vaccine.

27. A ComP glycosylation fragment comprising or consisting of an isolated fragment of a ComP protein,
wherein the ComP glycosylation fragment does not contain a cysteine residue corresponding to the conserved cysteine residue at position 71 of ComP₁₁₀₂₆₄ (SEQ ID NO: 1) and/or does not contain a cysteine residue corresponding to the conserved cysteine residue at position 93 of ComP₁₁₀₂₆₄ (SEQ ID NO: 1); and
wherein the ComP glycosylation fragment comprises the serine residue corresponding to the conserved serine residue at position 82 of ComP₁₁₀₂₆₄ (SEQ ID NO: 1);
optionally, wherein the ComP glycosylation fragment is immunogenic.

28. The ComP glycosylation fragment of claim 27, wherein the ComP glycosylation fragment has a length of from 5 to 22 amino acids in length, has a length of from 10 to 22 amino acids in length, has a length of from 11 to 22 amino acids in length, has a length of from 5 to 21 amino acids in length, has a length of from 10 to 21 amino acids in length, or has a length of from 11 to 21 amino acids in length;
optionally, wherein the fragment has at least 1, 2, 3, 4, 5, or 6 amino acid residues N-terminal to the serine residue corresponding to the conserved serine residue at position 82 of SEQ ID NO: 1 and/or wherein the fragment has at least 1, 2, 3, 4, 5, or 6 amino acid residues C-terminal to the serine residue corresponding to the conserved serine residue at position 82 of SEQ ID NO: 1.

29. The ComP glycosylation fragment of claim 27, wherein the amino acid sequence of the ComP glycosylation fragment does not extend in the N-terminus direction beyond the amino acid residue corresponding to position 72 of ComP₁₁₀₂₆₄ (SEQ ID NO: 1) and/or does not extend in the C-terminus beyond the amino acid residue corresponding to position 92 of ComP₁₁₀₂₆₄ (SEQ ID NO: 1)

30. The ComP glycosylation fragment of claim 27, wherein the ComP protein comprises an amino acid sequence that is at least 50%, 60%, 70%, 75%, 80%, 85%, 90%, 95%, 96%, 97%, 98%, 99%, or 100% identical to SEQ ID NO: 9 (ComPΔ28₁₁₀₂₆₄), SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59});
optionally, wherein the ComP protein comprises SEQ ID NO: 9 (ComPΔ28₁₁₀₂₆₄), SEQ ID NO: 10 (ComPΔ28_{ADP1}), SEQ ID NO: 11 (ComPΔ28_{GFJ-2}), SEQ ID NO: 12 (ComPΔ28_{P50v1}), SEQ ID NO: 13 (ComPΔ28₄₄₆₆), SEQ ID NO: 14 (ComPΔ28_{SFC}); SEQ ID NO: 15 (ComPΔ28_{P5312}), or SEQ ID NO: 16 (ComPΔ29_{ANT_H59}).

31. The ComP glycosylation fragment of claim 27, wherein the ComP glycosylation fragment comprises or consists of the amino acid consensus sequence of:
(SEQ ID NO: 17)
X₁GVX₄X₅X₆X₇X₈X₉ASX₁₂X₁₃TX₁₅NVX₁₈X₁₉X₂₀X₂₁
wherein: X₁ is V, T, A, or I;
X₄ is Q, T, E, A, or S;
X₅ is E, Q, T, or L;
X₆ is I or V;
X₇ is S, N, A, or G;
X₈ is S or no amino acid;
X₉ is G, D, or no amino acid;
X₁₂ is N, S, or A;
X₁₃ is A, S, or K;
X₁₅ is T, S, or K;
X₁₈ is A, E, Q, or L;
X₁₉ is T, S, or K;
X₂₀ is A or S; and
X₂₁ is T, Q, A, or V;
or a fragment of thereof of at least 5, 6, 7, 8, 9, 10, or 11 amino acids in length comprising the serine (S) residue at position 11 of SEQ ID NO: 17,
optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17;
or a variant of the amino acid consensus sequence of SEQ ID NO: 17 or the fragment thereof, having one, two, three, four, five, six, or seven amino acid substitutions, additions, and/or deletions, wherein the variant maintains the serine (S) residue at position 11 of SEQ ID NO: 17,
optionally, wherein the variant has at least 1, 2, 3, 4, 5, or 6 amino acid residues N-terminal to the serine (S) residue at position 11 of SEQ ID NO: 17 and/or wherein the variant has at least 1, 2, 3, 4, 5, or 6 amino acid residues C-terminal to the serine (S) residue at position 11 of SEQ ID NO: 17;
optionally, wherein the ComP glycosylation fragment can be glycosylated when located internally in a fusion protein; and
optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

32. The ComP glycosylation fragment of claim 27, wherein the ComP glycosylation fragment comprises or consists of the amino acid consensus sequence of:
(SEQ ID NO: 17)
X₁GVX₄X₅X₆X₇X₈X₉ASX₁₂X₁₃TX₁₅NVX₁₈X₁₉X₂₀X₂₁
wherein: X₁ is V, T, A, or I;
X₄ is Q, T, E, A, or S;
X₅ is E, Q, T, or L;
X₆ is I or V;
X₇ is S, N, A, or G;
X₈ is S or no amino acid;
X₉ is G, D, or no amino acid;
X₁₂ is N, S, or A;
X₁₃ is A, S, or K;
X₁₅ is T, S, or K;
X₁₈ is A, E, Q, or L;
X₁₉ is T, S, or K;
X₂₀ is A or S; and
X₂₁ is T, Q, A, or V;
or a fragment of thereof of at least 5, 6, 7, 8, 9, 10, or 11 amino acids in length comprising the serine (S) residue at position 11 of SEQ ID NO: 17,
optionally, wherein the fragment has at least 1, 2, 3, 4, 5, or 6 amino acid residues N-terminal to the serine (S) residue at position 11 of SEQ ID NO: 17 and/or wherein the fragment has at least 1, 2, 3, 4, 5, or 6 amino acid residues C-terminal to the serine (S) residue at position 11 of SEQ ID NO: 17;
optionally, wherein the ComP glycosylation fragment can be glycosylated when located internally in a fusion protein; and
optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

33. The ComP glycosylation fragment of claim 27, wherein the ComP glycosylation fragment comprises or consists of an amino acid sequence of SEQ ID NO: 32-163, or 164,
or a variant thereof having one, two, or three amino acid substitutions, additions, and/or deletions,
wherein the variant comprises the serine residue corresponding to the conserved serine (S) residue at position 82 of SEQ ID NO: 1;
optionally, wherein the ComP glycosylation fragment can be glycosylated when located internally in a fusion protein; and
optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

34. The ComP glycosylation fragment of claim 33, wherein the ComP glycosylation fragment comprises or consists of an amino acid sequence of:
iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATINVASA;
iGTccΔ2-1
(SEQ ID NO: 56)
GVTQIASGASAATTNVASAQ;
iGTccΔ2-2
(SEQ ID NO: 57)
GVTQIASGASAATINVASA;
iGTccΔ2-3
(SEQ ID NO: 58)
GVTQIASGASAATINVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN,
or the variant thereof.

35. The ComP glycosylation fragment of claim 33, wherein the ComP glycosylation fragment comprises or consists of an amino acid sequence of SEQ ID NO: 32-163, or 164,
optionally, wherein the ComP glycosylation fragment can be glycosylated when located internally in a fusion protein; and
optionally, wherein the ComP glycosylation fragment is not glycosylated when located at the N-terminal or C-terminal end of a fusion protein or is glycosylated at least 50% less, 60% less, 70% less, 80% less, 90% less, 95% less, or 99% less when located at the N-terminal or C-terminal end of a fusion protein in comparison to when it is located internally in the fusion protein.

36. The ComP glycosylation fragment of claim 35, wherein the ComP glycosylation fragment comprises or consists of an amino acid sequence of:
iGTccΔ0-1
(SEQ ID NO: 32)
CTGVTQIASGASAATTNVASAQ;
iGTccΔ1-0
(SEQ ID NO: 43)
TGVTQIASGASAATTNVASAQC;
iGTccΔ1-1
(SEQ ID NO: 44)
TGVTQIASGASAATTNVASAQ;
iGTccΔ1-2
(SEQ ID NO: 45)
TGVTQIASGASAATINVASA;
iGTccΔ2-1
(SEQ ID NO: 56)
GVTQIASGASAATTNVASAQ;
iGTccΔ2-2
(SEQ ID NO: 57)
GVTQIASGASAATINVASA;
iGTccΔ2-3
(SEQ ID NO: 58)
GVTQIASGASAATINVAS;
iGTccΔ3-2
(SEQ ID NO: 69)
VTQIASGASAATTNVASA;
iGTccΔ3-3
(SEQ ID NO: 70)
VTQIASGASAATTNVAS;
iGTccΔ3-4
(SEQ ID NO: 71)
VTQIASGASAATTNVA;
iGTccΔ4-3
(SEQ ID NO: 82)
TQIASGASAATTNVAS;
iGTccΔ4-4
(SEQ ID NO: 83)
TQIASGASAATTNVA;
iGTccΔ4-5
(SEQ ID NO: 84)
TQIASGASAATTNV;
iGTccΔ5-4
(SEQ ID NO: 95)
QIASGASAATTNVA;
iGTccΔ5-5
(SEQ ID NO: 96)
QIASGASAATTNV;
iGTccΔ5-6
(SEQ ID NO: 97)
QIASGASAATTN;
iGTccΔ6-5
(SEQ ID NO: 108)
IASGASAATTNV;
or
iGTccΔ6-6
(SEQ ID NO: 109)
IASGASAATTN,

37. A fusion protein comprising the ComP glycosylation fragment of claim 27, wherein the ComP glycosylation fragment is located internally within the fusion protein;
optionally, wherein the fusion protein is glycosylated by an oligo- or polysaccharide at a serine residue on the glycosylation fragment corresponding to the serine ComP glycosylation fragment residue at position 82 of SEQ ID NO: 1 (ComP₁₁₀₂₆₄).

38. The fusion protein of claim 37, wherein the oligo- or polysaccharide is derived from a saccharide produced by bacteria from the genus Streptococcus;
optionally, wherein the saccharide is a S. pneumoniae, S. agalactiae, or S. suis capsular polysaccharide;
optionally, wherein the saccharide is the serotype 8 capsular polysaccharide from S. pneumoniae;
optionally, wherein the saccharide is the type Ia, Ib, II, III, IV, V, VI, VII, VIII, or X capsular polysaccharide from S. agalactiae.

39. The fusion protein of claim 37, wherein the oligo- or polysaccharide is derived from a saccharide produced by the bacteria from the genus Klebsiella;
optionally, wherein the saccharide is a K. pneumoniae, K. varricola, K. michinganenis, or K. oxytoca capsular polysaccharide;
optionally, wherein the saccharide is a K. pneumoniae, K. varricola, K. michinganenis, or K. oxytoca O-antigen polysaccharide.

40. The fusion protein of claim 37, wherein oligo- or polysaccharide comprises glucose at its reducing end.

41. The fusion protein of claim 37, wherein the glycosylated fusion protein is produced in vivo;
optionally, in a bacterial cell;
optionally, in Escherichia coli;
optionally, in a bacterium from the genus Klebsiella;
optionally, wherein the bacterial species is K. pneumoniae, K. varricola, K. michinganenis, or K. oxytoca.

42. The fusion protein of claim 37, wherein the fusion protein comprises a carrier protein selected from the group consisting of Pseudomonas aeruginosa Exotoxin A (EPA), CRM₁₉₇, cholera toxin B subunit, tetanus toxin C fragment, Haemophilus influenzae Protein D, and a fragment or fragments thereof;
optionally, wherein the Pseudomonas aeruginosa Exotoxin A (EPA) carrier protein comprises the amino acid sequence of SEQ ID NO: 18, or a fragment or fragments thereof;
optionally, wherein the CRM₁₉₇ carrier protein comprises the amino acid sequence of SEQ ID NO: 24, or a fragment or fragments thereof.

43. The fusion protein of claim 42, wherein:
(i) the ComP glycosylation fragment is inserted between Ala489 and Arg490 relative to the PDB entity 1IKQ of Pseudomonas aeruginosa Exotoxin A (EPA) (SEQ ID NO: 19);
(ii) the ComP glycosylation fragment is inserted between Glu548 and Gly549 relative to the PDB entity 1IKQ of Pseudomonas aeruginosa Exotoxin A (EPA) (SEQ ID NO: 20);
(iii) the ComP glycosylation fragment is inserted between Ala122 and Gly123 relative to the PDB entity 1IKQ of Pseudomonas aeruginosa Exotoxin A (EPA) (SEQ ID NO: 21);
(iv) the ComP glycosylation fragment is inserted between Thr355 and Gly356 relative to the PDB entity 1IKQ of Pseudomonas aeruginosa Exotoxin A (EPA) (SEQ ID NO: 22); or
(v) the ComP glycosylation fragment is inserted between Lys20 and Asp21 relative to the PDB entity 1IKQ of Pseudomonas aeruginosa Exotoxin A (EPA) (SEQ ID NO: 23).

44. The fusion protein of claim 42, wherein:
(i) the ComP glycosylation fragment is inserted between Asn481 and Gly482 relative to the PDB entity 4AE0 of CRM₁₉₇ (SEQ ID NO: 25);
(ii) the ComP glycosylation fragment is inserted between Asp392 and Gly393 relative to the PDB entity 4AE0 of CRM₁₉₇ (SEQ ID NO: 26);
(iii) the ComP glycosylation fragment is inserted between Glu142 and Gly143 relative to the PDB entity 4AE0 of CRM₁₉₇ (SEQ ID NO: 27);
(iv) the ComP glycosylation fragment is inserted between Asp129 and Gly130 relative to the PDB entity 4AE0 of CRM₁₉₇ (SEQ ID NO: 28); or
(v) the ComP glycosylation fragment is inserted between Asn69 and Glu70 relative to the PDB entity 4AE0 of CRM₁₉₇ (SEQ ID NO: 29).

45. The fusion protein of claim 37, wherein the fusion protein comprises two or more, three or more, four or more, five or more, six or more, eight or more, ten or more, fifteen or more, or twenty or more ComP glycosylation fragments;
optionally, wherein the fusion protein does not comprise more than three, more than five, more than ten, more than fifteen, more than twenty, or more than twenty five ComP glycosylation fragments.

46. The fusion protein of claim 37, wherein the ComP glycosylation fragments are identical.

47. The fusion protein of claim 37, wherein the ComP glycosylation fragments differ from each other;
optionally, wherein at least three, at least four, or at least five of the ComP glycosylation fragments all differ from each other;
optionally, wherein none of the ComP glycosylation fragments are the same.

48. A method of in vivo conjugation of an oligo- or polysaccharide to an acceptor polypeptide, the method comprising covalently linking the oligo- or polysaccharide to the acceptor polypeptide with a PglS oligosaccharyltransferase (OTase), wherein the acceptor polypeptide comprises the ComP glycosylation fragment of claim 27.

49. The method of claim 48, wherein the PglS OTase is PglS₁₁₀₂₆₄ (SEQ ID NO: 165), PglS_{ADP1} (SEQ ID NO: 166), PglS_{GFJ-2} (SEQ ID NO: 167), PglS₅₀ᵥ₁ (SEQ ID NO: 168), PglS₄₄₆₆ (SEQ ID NO: 169), PglS_{SFC} (SEQ ID NO: 170), Pgl_{SP5312} (SEQ ID NO: 171), or PglS_{ANT_H59} (SEQ ID NO: 172).

50. The method of claim 48, wherein the oligo- or polysaccharide is linked to the ComP glycosylation fragment at a serine (S) residue corresponding to the serine residue at position 82 of SEQ ID NO: 1 (ComP₁₁₀₂₆₄).

51. The method of claim 48, wherein the in vivo conjugation occurs in a host cell.

52. The method of claim 51, wherein the host cell is a bacterial cell;
optionally, in Escherichia coli;
optionally, in a bacterium from the genus Klebsiella;
optionally, wherein the bacterial species is K. pneumoniae, K. varricola, K. michinganenis, or K. oxytoca.

53. The method of claim 51, comprising culturing a host cell that comprises: (a) a genetic cluster encoding for the proteins required to synthesize the oligo- or polysaccharide; (b) a PglS OTase; and (3) the acceptor polypeptide.

54. The method of claim 48, wherein production of the oligo- or polysaccharide is enhanced by the K. pneumoniae transcriptional activator rmpA (K. pneumoniae NTUH K-2044) or a homolog of the K. pneumoniae transcriptional activator rmpA (K. pneumoniae NTUH K-2044).

55. The method of claim 48, wherein the method produces a conjugate vaccine.

56. A host cell comprising (a) a genetic cluster encoding for the proteins required to synthesize an oligo- or polysaccharide; (b) a PglS OTase; and (3) an acceptor polypeptide comprising the ComP glycosylation fragment of claim 27.

57. The host cell of claim 56, wherein the acceptor polypeptide is a fusion protein.

58. The host cell of claim 56, wherein the host cell comprises a nucleic acid encoding the PglS OTase.

59. The host cell of claim 56, wherein the host cell comprises a nucleic acid encoding the acceptor polypeptide.

60. An isolated nucleic acid encoding the ComP glycosylation fragment of any one of claims 27 to 36 and/or the fusion protein of any one of claims 37 to 47.

61. The isolated nucleic acid of claim 60, wherein the nucleic acid is a vector.

62. A host cell comprising the isolated nucleic acid of claim 60 or 61.

63. A composition comprising the conjugate vaccine of any one of claims 21 to 26 or the fusion protein of any one of claims 37 to 47, and an adjuvant.

64. A method of inducing a host immune response against a bacterial pathogen, the method comprising administering to a subject in need of the immune response an effective amount of the conjugate vaccine of any one of claims 21 to 26, the fusion protein of any one of claims 37 to 47, or the composition of claim 63.

65. The method of claim 64, wherein the immune response is an antibody response.

66. The method of claim 64, wherein the immune response is selected from the group consisting of an innate response, an adaptive response, a humoral response, an antibody response, cell mediated response, a B cell response, a T cell response, cytokine upregulation or downregulation, immune system cross-talk, and a combination of two or more of said immune responses.

67. The method of claim 64, wherein the immune response is selected from the group consisting of an innate response, a humoral response, an antibody response, a T cell response, and a combination of two or more of said immune responses.

68. A method of preventing or treating a bacterial disease and/or infection in a subject comprising administering to a subject in need thereof the conjugate vaccine of any one of claims 21 to 26, the fusion protein of any one of claims 37 to 47, or the composition of claim 63.

69. The method of claim 68, wherein the infection is a localized or systemic infection of skin, soft tissue, blood, or an organ, or is auto-immune in nature.

70. The method of claim 69, wherein the disease is pneumonia.

71. The method of claim 69, wherein the infection is a systemic infection and/or an infection of the blood.

72. The method of any one of claims 68 to 71, wherein the subject is a human.

73. The method of any one of claims 68 to 72, wherein the composition is administered via intramuscular injection, intradermal injection, intraperitoneal injection, subcutaneous injection, intravenous injection, oral administration, mucosal administration, intranasal administration, or pulmonary administration.

74. A method of producing a pneumococcal conjugate vaccine against pneumococcal infection, the method comprising:
(a) isolating the glycoconjugate of any one of claims 1 to 26 or a glycosylated fusion protein of any one of claims 37 to 47; and
(b) combining the isolated glycoconjugate or isolated glycosylated fusion protein with an adjuvant.

75. The glycoconjugate, glycosylated fusion protein, or conjugate vaccine of any of the above claims for use in inducing a host immune response against a bacterial pathogen and/or preventing or treating a bacterial disease and/or infection in a subject.
